SEQUENCE LISTING (473 sequences)

SEQ ID NO: 1 - protein, 75 aa, Capsicum annuum
DICTTPPRRYIGVCWSDTSCRKVCVEMENFEDGYCSKIQRKCLCTWTCVLDNIPNDAGTIMLQDAKTMQLLEEKF

SEQ ID NO: 2 - protein, 75 aa, Capsicum annuum
YNICKTKSKYFEGLCWVDSSCRKVCIEKDKFEDGHCSKLLRNCLCTKICAFDNIPNDAGTILVQDAKSLEAQLLE

SEQ ID NO: 3 - protein, 75 aa, Capsicum annuum
QNNICKTTSKHFKGLCFADSKCRKVCIQEDKFEDGHCSKLQRKCLCTKNCVFDNIPNDVGTILVEDAKTLEAQLL

SEQ ID NO: 4 - protein, 75 aa, Capsicum annuum
QQTCKTESQKFKGLCITKSSCRKACLKEKFTDGHCSKLQRRCLCTKPCVFENNPNEVETKLVGKTKTLREAMLEE

SEQ ID NO: 5 - protein, 79 aa, Capsicum annuum
RQTCKTESQKFKGLCITKSSCRKACLKEKFTDGHCSKLQRRCLCTKPCVFENNPNEDETNLVGKTKTLREAMLEEQIME

SEQ ID NO: 6 - protein, 75 aa, Capsicum annuum
YNICKTKSKKFEGLCFLDSSCRKVCVEQENFEDGHCSKLRRLCLCTKLCVFDNIPNDVGTILVQDAKTLEAQLLE

SEQ ID NO: 7 - protein, 75 aa, Capsicum annuum
QNICKTKSKYFTGLCWTDSSCRKVCIEKDKFQDGHCSKLQRNCLCTKICVFDNIPNDAGTILVQDAKTNEAQLLE

SEQ ID NO: 8 - protein, 75 aa, Capsicum annuum; identical to SEQ ID NO: 2

SEQ ID NO: 9 - protein, 75 aa, Nicotiana tabacum
RECKTESNTFPGLCITKPPCRKACISEGFTDGHCSKILRRCLCTKPCVFDEKMIKTGAETLAEEAKTLAAALFEE

SEQ ID NO: 10 - protein, 75 aa, Nicotiana tabacum
RECKTESNTFPGLCITKPPCRKACISEGFTDGHCSKILRRCLCTKPCVFDEKMIKTGAETFAEEAKTLAAALLEE

SEQ ID NO: 11 - protein, 75 aa, Nicotiana tabacum
RECKAVSNTLSGLCITKPPCREACISEGFTDGKCSKILKRCICHKPCVFDEKMVKTGAETLAEEVKTLAAAFLEE

SEQ ID NO: 12 - protein, 75 aa, Nicotiana tabacum
RECKAESITFSGLCVSKPPCRKACICEGFTNGHCSKILRRCLCTTPSVFDEKMIKTGAETLGEEAKTLAATLLEE

SEQ ID NO: 13 - protein, 75 aa, Nicotiana tabacum
QSTCKAESNTFPGICITKPPCRKACLSEGFTDGKCSKILRRCICYKPCVFDEKMTKTGAETLAEEAKTLAAALLE

SEQ ID NO: 14 - protein, 75 aa, Nicotiana tabacum
RECKTESNTFPGICITKPPCRKACISEKFTDGHCSKLLRRCLCTKPCVFDEKMIKTGAETLVEEAKTLAAALLEE

SEQ ID NO: 15 - protein, 75 aa, Petunia hybrida
ATCKAECATWEGICINKAPCVKCCKEQPENFTDGHCSKILRRCLCTKPCATAEATATLANEVKTMGEALVEVDMM

SEQ ID NO: 16 - protein, 75 aa, Petunia hybrida
ATCKAECPTWDGICINKGPCVKCCKAQPEKFTDGHCSKVLRRCLCTKPCATEEATATLANEVKTMAEALVEEDMM

SEQ ID NO: 17 - protein, 75 aa, Petunia hybrida
ATCKAECPTWDSVCINKKPCVACCKKAKFSDGHCSKILRRCLCTKECVFEKTEATQTETFTKDVNTLAEALLEAD

SEQ ID NO: 18 - protein, 75 aa, Solanum lycopersicum
QQICKAPSQTFPGLCFMDSSCRKYCIKEKFTGGHCSKLQRKCLCTKPCVFDKISSEVKATLGEEAKTLSEVVLEE

SEQ ID NO: 19 - protein, 68 aa, Solanum lycopersicum
QPMCKSTSQTFKGLCFTDSSCRKSCLKEEFEGGHCSKLQRKCLCTKICVFDKISNEVRTNFGWGSKNS

SEQ ID NO: 20 - protein, 78 aa, Solanum lycopersicum
ASSTRANQQAKSSKDYVLPTHHVEKLVSKRSLKVDIIFKLQSKCLCTKICVFEKNPNEVKPTLGGEAKTLSETMLEDD

SEQ ID NO: 21 - protein, 62 aa, Solanum lycopersicum
QHMCKSTSQTFKGLCFTYSSCRKGCLKEKFEGGHYIVANSKGSAYALRFVYLTKFQMKLNQL

SEQ ID NO: 22 - protein, 75 aa, Solanum lycopersicum
QYMCKSTSQTFKGLCFTDSSCRKACVTEEFTGGHCSKLQRKCLCTKVCVLEKDSNEVKTTLVGEAKTLSETVLEE

SEQ ID NO: 23 - protein, 75 aa, Solanum lycopersicum
QQMCKSTSQTFKGLCFTDSSCRKACVTEEFTGGHCSKLQRKCLCTKVCVFEKDSNEVKTTLVGEAKTLSETVLEE

SEQ ID NO: 24 - protein, 75 aa, Solanum pennellii
RQICKAPSQTFPGLCFMDSSCRKYCIKEKFTGGHCSTLQRRCLCTKPCVFDIISNEVKATLGEEAKTLSEVVLEE

SEQ ID NO: 25 - protein, 75 aa, Solanum pennellii
QQMCKSTSQTFKGLCFIDSSCRKACVTEEFTGGHCSKLQRKCLCTKVCVFEKDSNEVKTTLVGEAKTLSETVLEE

SEQ ID NO: 26 - protein, 49 aa, Cajanus cajan
AVCSKPSTYFFGPCVRRSTCRRACSHENYPDGKCSRFLGKCICYKPCTT

SEQ ID NO: 27 - protein, 46 aa, Eucalyptus grandis
ATCRKPSMYFSGACFSDTNCQKACNREDWPNGKCLVGFKCECQRPC

SEQ ID NO: 28 - protein, 55 aa, Eucalyptus grandis
MNIVTKVDGAICKKPSKFFKGACGRDADCEKACDQENWPGGVCVPFLRCECQRSC

SEQ ID NO: 29 - protein, 49 aa, Eucalyptus grandis
ATCAKPSKYYKGGCFATISVSACKKTCSRENWPDGACVYPFRCECRRPC

SEQ ID NO: 30 - protein, 49 aa, Eucalyptus grandis
KTCAKPSKYYKGGCFSTISVSACKKTCSRENWPDGDCVFPYRCECRRPC

SEQ ID NO: 31 - protein, 49 aa, Eucalyptus grandis
ANCAKPSKFYRGPCFNSISVKPCTQRCSQENWPIGVCTKDLRCECQRPC

SEQ ID NO: 32 - protein, 49 aa, Eucalyptus grandis
FSVISDSACKKACNQENWPIGICVPVFRCECRRSCQAMFSPESSSIKYE

SEQ ID NO: 33 - protein, 49 aa, Brassica napus
KARMCETSSQLFNGPCLSTTNCANICQNEGFPDGDCKGFRLRCICNRPC

SEQ ID NO: 34 - protein, 47 aa, Brassica rapa
RTCESQSHRFKGPCVSENNCANVCHNEGFGGGKCRGLRRRCFCTRHC

SEQ ID NO: 35 - protein, 50 aa, Brassica napus
IEGRMCQSRSHHFRSICLSNHNCANICRGERFSGGRCHGLHRKCYCTRLC

SEQ ID NO: 36 - protein, 45 aa, Brassica rapa
RTCESPSTKFQGVCLNAKNCAGVCTGEGFTGGQCSSLKCYCTKTC

SEQ ID NO: 37 - protein, 47 aa, Brassica cretica
RTCESKSHRFKGTCVSSTNCGNVCHNEGFGGGKCRGFRRRCYCTRHC

SEQ ID NO: 38 - protein, 47 aa, Brassica napus
RTCESKSHKFRGPCVSRHNCANVCHNEGFHGGKCRGFRRRCYCTRHC

SEQ ID NO: 39 - protein, 48 aa, Zostera marina
RTCVSKSEKFKGVCLINTNCAHVCQTEGFPDGDCRGSVRRRCFCIRPC

SEQ ID NO: 40 - protein, 47 aa, Cajanus cajan
RTCESKSHRFKGPCVSTHNCGNVCHNEGFGGGKCRGFRRRCYCTRHC

SEQ ID NO: 41 - protein, 47 aa, Arabidopsis lyrata
RTCASKSEKFKGSCVSNTNCANICHTEGFPEGACRGLRHHCFCTRPC

SEQ ID NO: 42 - protein, 53 aa, Cajanus cajan
QVRMCESKSHEFKGFCTKWRICRDVCITEGFTDGGCHGVLRHCVCKKPCLLSN

SEQ ID NO: 43 - protein, 54 aa, Actinidia chinensis
MIKQGEARVCESKSHGFKGACLIDHNCALVCRNEGFSGGKCRGFRHRCFCTKIC

SEQ ID NO: 44 - protein, 63 aa, Actinidia chinensis
KLCTAPSSTFKYLCISDQNCSSACEQEGFTSGECEGLRRRCICGKPCGMEVSKTKFDNGIGGE

SEQ ID NO: 45 - protein, 47 aa, Arabidopsis lyrata
RTCETSSNLFNGPCLSSSNCANVCHNEGFSDGDCRGFRRRCLCTRPC

SEQ ID NO: 46 - protein, 54 aa, Arabidopsis lyrata
QPQLCETKSLNYRGLCMKWRNCKRVCISEGFPDGRCKGFFNNKCVCRKPCALST

SEQ ID NO: 47 - protein, 47 aa, Arabidopsis lyrata
RTCASQSQRFKGKCVSDTNCANVCHNEGFPGGDCRGFRRRCFCTRNC

SEQ ID NO: 48 - protein, 47 aa, Arabidopsis lyrata
RTCESKSHRFKGPCVSTHNCANVCHNEGFGGGKCRGFRRRCYCTRHC

SEQ ID NO: 49 - protein, 45 aa, Arabidopsis lyrata
RTCESPSNKFQGVCLNSQSCAKACPSEGFSGGRCSSLRCYCSKAC

SEQ ID NO: 50 - protein, 59 aa, Arabidopsis lyrata
EVSPVDNKICKTRSDRFSGVCLSTNNCAIICQQFEHFDGGHCEFDGALRRCMCTKQCNN

SEQ ID NO: 51 - protein, 54 aa, Arabidopsis lyrata; identical to SEQ ID NO: 46

SEQ ID NO: 52 - protein, 52 aa, Arabidopsis lyrata
QQMCETQSINFRGMCTKWRNCQRVCMSEGFPDGRCKGFIRKCICTKPCPVST

SEQ ID NO: 53 - protein, 53 aa, Arabidopsis lyrata
IIGSDGRTCQSKSHHFKYMCTSSHNCAIVCRNEGFSGGRCHGFHRRCYCTRLC

SEQ ID NO: 54 - protein, 49 aa, Cajanus cajan
RDCESQSHKFKGTCIRKSNCASVCQSEGFNGGHCRGVTRRCYCTAKCHE

SEQ ID NO: 55 - protein, 49 aa, Arachis hypogaea
GGRHCESKSHRFKGMCVHERNCASVCNLEGFSGGKCRGFRRRCFCTRHC

SEQ ID NO: 56 - protein, 63 aa, Arachis hypogaea
EGRKCDSQSHHFKGKCFSDTNCASVCHGSFIHVFVELDLQSFEFTSCFSLEIKKFSSRSGQLF

SEQ ID NO: 57 - protein, 48 aa, Arachis hypogaea
RTCASQSHRFKGVCLSDTNCASVCKTEGFPSGDCHGFRRRCFCTKHCA

SEQ ID NO: 58 - protein, 55 aa, Arachis hypogaea
RTCASQSHRFKGVCLSDTNCASVCKTEGFPSGDCHGFRRRCFCTKHYRKIRSQDS

SEQ ID NO: 59 - protein, 49 aa, Arachis hypogaea
EGRHCESKSHRFKGMCVHERNCASVCNLEGFSGGKCRGFRRRCFCTRHC

SEQ ID NO: 60 - protein, 47 aa, Arachis hypogaea
RVCQSKSHMFKGGCWGDHNCALVCRNEGFSGGRCRGFRRRCFCTKLC

SEQ ID NO: 61 - protein, 47 aa, Arabidopsis thaliana; identical to SEQ ID NO: 45

SEQ ID NO: 62 - protein, 47 aa, Arabidopsis thaliana
RTCESQSHRFKGTCVSASNCANVCHNEGFVGGNCRGFRRRCFCTRHC

SEQ ID NO: 63 - protein, 47 aa, Arabidopsis thaliana
RTCASQSQRFKGKCVSDTNCENVCHNEGFPGGDCRGFRRRCFCTRNC

SEQ ID NO: 64 - protein, 47 aa, Arabidopsis thaliana; identical to SEQ ID NO: 48

SEQ ID NO: 65 - protein, 45 aa, Arabidopsis thaliana; identical to SEQ ID NO: 49

SEQ ID NO: 66 - protein, 57 aa, Arabidopsis thaliana
EVSPLDNKICKTRSDRFSGVCISTNNCAIICQQFEHFDGGHCEFDGAFRRCMCTKQC

SEQ ID NO: 67 - protein, 57 aa, Arabidopsis thaliana
QPQLCETKSLNYRGLCLKWRSCKRVCISEGFPDGRCKGFFNNKCVCRKPCALLSTEN

SEQ ID NO: 68 - protein, 53 aa, Arabidopsis thaliana
QQMCEAKSLDWKGMCLKWRNCRQVCISEGFTDGRCKGFTRKCICSKPCFVLPN

SEQ ID NO: 69 - protein, 47 aa, Arabidopsis thaliana
RTCQSKSHHFKYMCTSNHNCAIVCRNEGFSGGRCHGFHRRCYCTRLC

SEQ ID NO: 70 - protein, 58 aa, Cajanus cajan
EMGQNQMVAEARTCESQSHKFKGTCVSKSNCAHVCQTEGFPGGHCRGFRRRCFCAKNC

SEQ ID NO: 71 - protein, 55 aa, Cajanus cajan
QNEMVAEARTCESQSHKFKGTCLSKSNCANVCKTEGFPGGHCRGFRRRCFCTKKC

SEQ ID NO: 72 - protein, 49 aa, Brassica carinata
KARMCETSSQLFNGPCLSTTNCANICQNEGFPDGDCHGFRLRCICNRPC

SEQ ID NO: 73 - protein, 47 aa, Brassica carinata
RTCESQSHRFKGTCVSENNCANVCHNEGFGGGKCRGFRRRCFCTRNC

SEQ ID NO: 74 - protein, 75 aa, Brassica carinata
MDPVTVEARTCESKSHKFRGLCVSTHNCANVCHTKVKRSKECVSTDRTVPLLVPKKVPQAVVAVVSVAPAPNPAS

SEQ ID NO: 75 - protein, 47 aa, Brassica carinata; identical to SEQ ID NO: 38

SEQ ID NO: 76 - protein, 47 aa, Brassica carinata
RTCESQSHRFKGPCVSDNNCANVCHNEGFGGGKCRGLRRRCFCTTHC

SEQ ID NO: 77 - protein, 47 aa, Brassica carinata
RTCESKSHKFRGLCVSRHNCANVCHNEGFHGGKCRGFRRRCYCTRHC

SEQ ID NO: 78 - protein, 47 aa, Brassica carinata
RTCESKSHRFKGTCVSTTNCANVCHNEGFPGGKCRGFRRRCFCTRHC

SEQ ID NO: 79 - protein, 47 aa, Brassica carinata; identical to SEQ ID NO: 37

SEQ ID NO: 80 - protein, 49 aa, Brassica carinata; identical to SEQ ID NO: 33

SEQ ID NO: 81 - protein, 49 aa, Brassica oleracea; identical to SEQ ID NO: 33

SEQ ID NO: 82 - protein, 47 aa, Brassica oleracea
RTCESQSHRFKGPCVSDNNCANVCHNEGFGGGKCRGLRRRCFCTRHC

SEQ ID NO: 83 - protein, 50 aa, Brassica oleracea; identical to SEQ ID NO: 35

SEQ ID NO: 84 - protein, 72 aa, Brassica oleracea
QQMCEAKSLNFKGMCLKWKNCKHVFFTRKQLIKLSKKDVAAAEEEKILKETCMKLVQGQQMCEAKSINFKGM

SEQ ID NO: 85 - protein, 47 aa, Brassica oleracea; identical to SEQ ID NO: 37

SEQ ID NO: 86 - protein, 47 aa, Brassica oleracea; identical to SEQ ID NO: 77

SEQ ID NO: 87 - protein, 49 aa, Brassica rapa; identical to SEQ ID NO: 33

SEQ ID NO: 88 - protein, 47 aa, Brassica rapa; identical to SEQ ID NO: 34

SEQ ID NO: 89 - protein, 47 aa, Brassica rapa
RMCQSRSHHFRSICLSNHNCANICRGERFSGGRCHGLHRKCYCTRLC

SEQ ID NO: 90 - protein, 47 aa, Brassica rapa; identical to SEQ ID NO: 37

SEQ ID NO: 91 - protein, 47 aa, Brassica rapa; identical to SEQ ID NO: 38

SEQ ID NO: 92 - protein, 49 aa, Corchorus olitorius; identical to SEQ ID NO: 33

SEQ ID NO: 93 - protein, 47 aa, Corchorus olitorius; identical to SEQ ID NO: 82

SEQ ID NO: 94 - protein, 50 aa, Corchorus olitorius; identical to SEQ ID NO: 35

SEQ ID NO: 95 - protein, 72 aa, Corchorus olitorius
QQMCEAKSINFKGMSLKWKNCKHFIHENELIKLSRKEVAAAEEEKFLKETCMKLVQGQQMCEAKSINFKGIC

SEQ ID NO: 96 - protein, 47 aa, Corchorus olitorius; identical to SEQ ID NO: 37

SEQ ID NO: 97 - protein, 45 aa, Corchorus olitorius; identical to SEQ ID NO: 36

SEQ ID NO: 98 - protein, 47 aa, Corchorus olitorius; identical to SEQ ID NO: 77

SEQ ID NO: 99 - protein, 48 aa, Capsicum annuum
RSCESASQRFNGYCVRSSNCASICSTEGFTDGKCKGTIRRHCTCIRNC

SEQ ID NO: 100 - protein, 48 aa, Capsicum annuum
RVCISQSHGFKGPCGHDHNCALVCRNEGFSGGDCIGVFIRKCYCTKNC

SEQ ID NO: 101 - protein, 48 aa, Capsicum annuum
KICEALSGNFKGLCLSSRDCGNVCRREGFTDGSCIGFRLQCFCTKPCA

SEQ ID NO: 102 - protein, 47 aa, Capsicum annuum
RKCESQSHRFKGPCVRKSNCASVCQTEGFTGGHCRGFRRRCFCTKHC

SEQ ID NO: 103 - protein, 47 aa, Capsicum annuum
RHCESKSQRFKGPCVRQKNCAAVCETEGFPGGDCRGFRRRCFCTRPC

SEQ ID NO: 104 - protein, 47 aa, Capsicum annuum
RTCESQSHRFKGVCASETNCASVCQTEGFSGGDCRGFRRRCFCTRPC

SEQ ID NO: 105 - protein, 51 aa, Capsicum annuum
KTCDTLSENFKGICFSSTNCATTCQNEDEEYTGGHCRGLHRRCFCTTECEL

SEQ ID NO: 106 - protein, 49 aa, Capsicum annuum
KTCERLSGTFKGICFSSKNCASVCENEDEEFTGGHCSGLRRRCYCTADC

SEQ ID NO: 107 - protein, 47 aa, Cannabis sativa
RVCESKSHHFKGLCVIDHNCATICRTEGFSGGNCQGFRQRCFCTRHC

SEQ ID NO: 108 - protein, 47 aa, Cannabis sativa
RTCEAQSHHFKGPCVRKSNCANVCRTEGFPSGHCRGFRRRCFCTKHC

SEQ ID NO: 109 - protein, 47 aa, Cannabis sativa
KTCEARSQSHRGICVTNHTCDNVCKIERFSSGYCSGIDRRCLCTKHC

SEQ ID NO: 110 - protein, 47 aa, Cardamine hirsuta
RTCQSSSQLFSGPRLSTSNCANVCQNEGFTDGDCRGFRRRCFCNKQC

SEQ ID NO: 111 - protein, 54 aa, Cardamine hirsuta
QQLCETKSTNYRGLCLRWRNCKRVCMSEGFPDGRCRGIFDNKCVCRKPCALSTA

SEQ ID NO: 112 - protein, 47 aa, Cardamine hirsuta
RTCESQSHRFKGTCVSASNCGNVCHNEGFNGGNCRGFRRRCFCTRHC

SEQ ID NO: 113 - protein, 47 aa, Cardamine hirsuta
RTCESQSHRFKGTCTNEQNCRNICHNEGFFGGKCRGFRRRCFCTRNC

SEQ ID NO: 114 - protein, 47 aa, Cardamine hirsuta
RMCESKSQQFRGLCMSSTNCATVCHNEGFPGGECRGFRRRCFCTRPC

SEQ ID NO: 115 - protein, 100 aa, Cardamine hirsuta
KPRRCESQSNKFKGKCVSETNCGNVCRTEGFTGGDCRGFRRRCFCTRNCCARHRMKLSMRLISAALLLLMLFVATGMGPVMVDARTCESKSHRFKGPCVS

SEQ ID NO: 116 - protein, 47 aa, Cardamine hirsuta
RVCETRSQKFKGPCVSENNCANVCQTEGFPDGDCNGILRRCYCNSPC

SEQ ID NO: 117 - protein, 45 aa, Cardamine hirsuta
KTCETPSTKFQGVCLNSQNCAKVCPTEGASGGRCNSLRCVCSKTC

SEQ ID NO: 118 - protein, 47 aa, Cardamine hirsuta
RICQSKSHHYKYMCTSNHNCAMVCRNEGFSGGQCHGFHRRCYCTRLC

SEQ ID NO: 119 - protein, 47 aa, Cajanus cajan
RTCESSSNLFSGPCLSESNCANVCNTEGFPDGDCRGFRRRCMCTRPC

SEQ ID NO: 120 - protein, 47 aa, Cajanus cajan
RTCESKSHRFKGPCVSGNNCKNVCHNEGFPGGRCRGFRRRCYCTRHC

SEQ ID NO: 121 - protein, 45 aa, Cajanus cajan
RTCESPSSKFSGVCLNSQTCAKACPSEGFTGGRCNSLRCYCSKPC

SEQ ID NO: 122 - protein, 57 aa, Cajanus cajan
EMGLAADKVCKTRSDRFSGVCLSNNNCAIICQQFENFDSGHCEFDGAFRRCLCTKNC

SEQ ID NO: 123 - protein, 47 aa, Cajanus cajan
RMCQSKSHHFRRMCWSDHNCAMICRSERFSGGHCHGFHRRCYCTRLC

SEQ ID NO: 124 - protein, 47 aa, Cajanus cajan
RVCASQSHAFKGPCVGDHNCALVCRNEGFSGGNCQGFRRRCFCTKLC

SEQ ID NO: 125 - protein, 47 aa, Cajanus cajan
RTCESLSHRFKGTCVRGSNCAAVCQTEGFPGGQCSGLRRRCFCSKPC

SEQ ID NO: 126 - protein, 77 aa, Cajanus cajan
HDTEMGPVLCGDTGNEKGPSRRKAAAVAAIERTCETPSKRFKGPCVKESNCAAVCQTEGFQGGKCRGLRRRCFCTRP

SEQ ID NO: 127 - protein, 47 aa, Cajanus cajan
RICESQSHRFKGPCIRKDNCAAVCQTEGFHGGRCRGFRRRCFCTKHC

SEQ ID NO: 128 - protein, 47 aa, Cajanus cajan
RMCESPSRHYKGLCFRSSNCASICHIEHFSHGHCHGFRRRCICSRPC

SEQ ID NO: 129 - protein, 47 aa, Cajanus cajan
RMCESPSRHYKGLCFRSSNCASICHIEHFSHGYCHGFRRRCICSRPC

SEQ ID NO: 130 - protein, 47 aa, Cajanus cajan
RVCESQSHKFHGPCLGDHNCALVCRNEGFSGGDCKGLRRRCFCTRLC

SEQ ID NO: 131 - protein, 50 aa, Citrullus lanatus
AEARICESQSHRFKGPCVSKSNCAAVCQTEGFHGGHCRGFRRRCFCTKRC

SEQ ID NO: 132 - protein, 47 aa, Citrullus lanatus
RVCQSQSHHFHGACFSHHNCAFVCRNEGFSGGKCRGARRRCFCSKLC

SEQ ID NO: 133 - protein, 47 aa, Carya illinoinensis
RVCESKSHGFQGMCTRDHNCALVCRNEGFSGGKCRGFRHRCFCTKLC

SEQ ID NO: 134 - protein, 47 aa, Carya illinoinensis
RVCESKSHKFQGPCVRDNNCGLVCKNEGFTGGKCRGFRRRCFCTKIC

SEQ ID NO: 135 - protein, 58 aa, Citrullus lanatus
EEMMGGGRAEGRVCKSQSHSFHGPCMRDHNCAVVCRTEGFSGGDCVGFRRRCFCTRLC

SEQ ID NO: 136 - protein, 47 aa, Corchorus olitorius
RVCESKSHWFKGPCWGDHNCALVCRNEGFSGGRCRGFRHRCFCTKLC

SEQ ID NO: 137 - protein, 47 aa, Corchorus olitorius
RLCQSQSHGFKGGCLSNHNCALVCRNEGFSGGQCRGLRHRCFCTRRC

SEQ ID NO: 138 - protein, 32 aa, Carica papaya
RTCQSQSHGFHGACLSHHNCAVVCQKEGFSGX

SEQ ID NO: 139 - protein, 47 aa, Carica papaya
RTCESQSHRFKGACGSRSNCAAVCQTEGFHGGHCRGFRRRCFCTKHC

SEQ ID NO: 140 - protein, 50 aa, Cucumis sativus
VEGRICESKSHGFKGPCMTDHNCALVCRNEGFSGGKCRGVRHRCFCTKLC

SEQ ID NO: 141 - protein, 50 aa, Cucumis sativus; identical to SEQ ID NO: 140

SEQ ID NO: 142 - protein, 73 aa, Daucus carota
TDTCEVRSAYFVSLCLIDHHCEMVCKVEGFPTGKCLGLVPHCICLGTCKQPSPPSPPPANPPESPPPVPPPTP

SEQ ID NO: 143 - protein, 47 aa, Daucus carota
RTCESQSHKFKGTCLSETNCGNVCKNEGFSGGNCRGLRRRCFCTRHC

SEQ ID NO: 144 - protein, 47 aa, Daucus carota
RTCESQSHKFKGTCVSRSNCANVCKNEGFPGGHCRGFRRRCFCIKHC

SEQ ID NO: 145 - protein, 47 aa, Cajanus cajan
RVCEAQSHGFKGACMSNHNCALVCRNEGFSGGKCRGFRHRCFCTKQC

SEQ ID NO: 146 - protein, 47 aa, Cajanus cajan
RVCESKSHRFKGPCLSHHNCGMVCKNEGFSGGKCRGVRRRCFCTRRC

SEQ ID NO: 147 - protein, 52 aa, Cajanus cajan
ISVEGRTCESQSHRFKGACVSENNCAAVCQTEGFLGGNCRGFRRRCFCTKQC

SEQ ID NO: 148 - protein, 52 aa, Cajanus cajan
IPLEGRTCESQSHRFKGMCVRESNCATVCQTEGFQGGHCRGFHRRCFCTKHC

SEQ ID NO: 149 - protein, 47 aa, Cajanus cajan
RTCESQSHRFKGTCLRKSNCAAVCQTEGFHGGHCRGFRRRCFCTKHC

SEQ ID NO: 150 - protein, 50 aa, Cajanus cajan
GEARVCESKSHKYKGACFHDHNCAYVCRNEGFSGGKCHGLHHHCFCTKIC

SEQ ID NO: 151 - protein, 47 aa, Cajanus cajan
RVCESKSHKFKGPCMGDHNCAYVCRNEGFSGGKCRGLRHRCFCTKIC

SEQ ID NO: 152 - protein, 47 aa, Cajanus cajan
RMCQSQSHMYHGACLRDHNCALVCRNEGFSGGRCRGFRRRCFCTRLC

SEQ ID NO: 153 - protein, 71 aa, Cajanus cajan
NEMGGPMVVEGRTLAVVTVIGAGGVQRWWRWWQCNGDSGGDGGTRAMVAVTNSGGSSSNGGGGDDWWWWWQ

SEQ ID NO: 154 - protein, 48 aa, Cajanus cajan
RMCTNPSKTYKGPCVSDTNCESVCQSEGFPQGNCHGFRRRCICGKPCA

SEQ ID NO: 155 - protein, 47 aa, Cajanus cajan
RTCETASHKFKGPCLGDRNCANVCQTEGFPGGDCQGLRRRCFCTRPC

SEQ ID NO: 156 - protein, 48 aa, Cajanus cajan
RTCDAPSHSFKGICTRKRNCESVCNSEGFADGDCKGFRRRCICNRPCA

SEQ ID NO: 157 - protein, 49 aa, Cajanus cajan
RTCVTPSHRFRGICVSKRNCESACNSEGFPDGNCKGFRRRCICNRPCAK

SEQ ID NO: 158 - protein, 51 aa, Cajanus cajan
LFGRCKKVPSRTYKGPCARDANCRSVCISEGYNGGDCHGFRRRCMCTDKCS

SEQ ID NO: 159 - protein, 47 aa, Eucalyptus grandis
RTCESQSQRFKGACVSKTNCASVCQTEGFHGGHCRGFRRRCFCTKHC

SEQ ID NO: 160 - protein, 50 aa, Eucalyptus grandis
IEGRMCESQSHKFHGLCMSDRTCVVVCKEEDFDDGKCRGFRHRCFCRKPC

SEQ ID NO: 161 - protein, 47 aa, Eucalyptus grandis
RVCESQSHGFHGSCVSGHNCALVCRNEGFSGGRCRGFRRRCFCTKLC

SEQ ID NO: 162 - protein, 47 aa, Cajanus cajan
RVCESQSHGFHGTCIRAHNCALVCRHEGFSGGRCRGFRRRCFCTRRC

SEQ ID NO: 163 - protein, 47 aa, Cajanus cajan
RTCETLSHKFKGTCLRESNCASVCQTEGFTGGDCRGLRRRCFCTKHC

SEQ ID NO: 164 - protein, 47 aa, Cajanus cajan
RVCESQSHGFYGTCIHAHNCALVCRHEGFSGGRCRGFRRRCFCTRRC

SEQ ID NO: 165 - protein, 47 aa, Cajanus cajan
AICKVPSKTFKGMCFSQKNCIVRCHSEGYGWGECSHLRRRCMCYKPC

SEQ ID NO: 166 - protein, 47 aa, Cajanus cajan
RVCEVPSKTFTGLCFRDTHCIVRCHSEGYGYGKCSHVLRRCRCLKAC

SEQ ID NO: 167 - protein, 47 aa, Cajanus cajan; identical to SEQ ID NO: 165

SEQ ID NO: 168 - protein, 47 aa, Cajanus cajan; identical to SEQ ID NO: 162

SEQ ID NO: 169 - protein, 53 aa, Cajanus cajan
GRPDPKTCESPSAKFEGPCIMGSNCALTCQKEGHDGGFCRDFWLPKCFCTKPC

SEQ ID NO: 170 - protein, 47 aa, Cajanus cajan
RTCESLSHKFQGTCLRESNCASVCQTEGFTGGDCRGLRRRCFCTKHC

SEQ ID NO: 171 - protein, 53 aa, Cajanus cajan
GRPDPKTCEPPSAKFEEPCIMGSNCALTCQKEGHDGGFCHDFWLPKCFCTKPC

SEQ ID NO: 172 - protein, 75 aa, Cajanus cajan
LGPKVMVAHEAITCETQSIKYEGMCSREKPCASLCQTKGYSRGECHEVITLRLLYRPGKTDPIGQSKRRTYPLTN

SEQ ID NO: 173 - protein, 47 aa, Cajanus cajan; identical to SEQ ID NO: 166

SEQ ID NO: 174 - protein, 53 aa, Cajanus cajan
GRPDPKTCESPSAKFEGPCIMGSNCALTCQKEGHNGGFCRDFWLPKCFCTKPC

SEQ ID NO: 175 - protein, 53 aa, Cajanus cajan
GRPDPKTCESPSGKFEGPCIMGSNCALTCQKEGHDGGFCRDFWLPKCFCTKPC

SEQ ID NO: 176 - protein, 53 aa, Cajanus cajan; identical to SEQ ID NO: 169

SEQ ID NO: 177 - protein, 58 aa, Cajanus cajan
LGPKVMVAHEAITCETQSIKYEGMCSREKPCASLCQTKGYSRGECHGVYKICVCATQC

SEQ ID NO: 178 - protein, 47 aa, Cajanus cajan; identical to SEQ ID NO: 165

SEQ ID NO: 179 - protein, 48 aa, Cajanus cajan
SICEIPSKKFKGLCIRDRKCIVICHSEGYGYGKCSHIGRRCMCLKTCD

SEQ ID NO: 180 - protein, 54 aa, Cajanus cajan
MVAADDARKCESLSHNFNGLCLSESNCASVCATEGFTGGDCRGLRRRCFCTKQC

SEQ ID NO: 181 - protein, 47 aa, Cajanus cajan; identical to SEQ ID NO: 163

SEQ ID NO: 182 - protein, 47 aa, Cajanus cajan
RTCESLSHKFKGTCLSETNCASVCKTEGFSGGDCRGLRRRCFCTKHC

SEQ ID NO: 183 - protein, 47 aa, Cajanus cajan; identical to SEQ ID NO: 162

SEQ ID NO: 184 - protein, 45 aa, Glycine max
MHLRSHRFKGMCLSEHNCASVSHLEGFTGGKCWGFRRRCFCSKHC

SEQ ID NO: 185 - protein, 47 aa, Glycine max
KDCLTRRHGFQGRCLFDRQCAHVCRSDGFIGGQCRGPLRKCFCSRPC

SEQ ID NO: 186 - protein, 47 aa, Glycine max
RVCESQSHGFHGLCNRDHNCALVCRNEGFSGGRCKGFRRRCFCTRIC

SEQ ID NO: 187 - protein, 47 aa, Glycine max
RHCESKSHRFKGMCLSKHNCASVCHLEGFTGGKCRGFRRRCFCTRHC

SEQ ID NO: 188 - protein, 63 aa, Glycine max
RYIYIIPYVAVKRAEGRDCSSKSHGFKGLCFSDTNCAHVCRTEGFTGGDCGGFRRRCFCTRIC

SEQ ID NO: 189 - protein, 69 aa, Glycine max
RTCESQSHRFKGPCLSDTNCGSVCRTEGFSGGHCRGFSRRCFCTKEYDNYYKSSDVIRCKDGSGKFTKA

SEQ ID NO: 190 - protein, 47 aa, Glycine max
RTCESQSHRFKGPCLSDTNCGSVCRTERFTGGHCRGFRRRCFCTKHC

SEQ ID NO: 191 - protein, 48 aa, Gossypium hirsutum
GPICKATSNVYKGLCLIDDSCDIVCKREGFHKGNCKGFLRKCVCTNPC

SEQ ID NO: 192 - protein, 48 aa, Gossypium hirsutum
KTCETKSGKFKGMCMSSTNCASVCKSEPGFDGGHCRGFRRQCVCTKPC

SEQ ID NO: 193 - protein, 48 aa, Gossypium hirsutum
TTCETQSGKFKGMCMSSTNCASVCKSEPGFDGGYCRGFRRQCFCTKPC

SEQ ID NO: 194 - protein, 48 aa, Gossypium hirsutum
RTCETKSSEYKGICLFDANCDSICKVEPSFDGGHCHGFFRRCYCTKPC

SEQ ID NO: 195 - protein, 48 aa, Gossypium hirsutum
RTCETKSSEYKGICLFDANCDSVCKVEPGFDGGHCHGFFRRCYCTKPS

SEQ ID NO: 196 - protein, 51 aa, Gossypium hirsutum
MEDRICQSRSTEYRGMCLFDVNCDNICKTEPGFTGGHCHSFFRSCYCTKPC

SEQ ID NO: 197 - protein, 48 aa, Gossypium hirsutum
RKCESPSHKFKGMCMNRDNCAIVCQTEGYEDGKCEGFRRRCICIKPCF

SEQ ID NO: 198 - protein, 55 aa, Gossypium hirsutum
GPTIVGGRKCDSPSKKFNGMCMNHDNCATVCQTESHEDGKCEGFYRRCICIKPCF

SEQ ID NO: 199 - protein, 52 aa, Gossypium hirsutum
GVVEGRICESKSHRFKGVCLSDHNCGLVCRNEGFLDGWCRGFRHRCFCTRNC

SEQ ID NO: 200 - protein, 47 aa, Gossypium hirsutum
RVCQSQSHKFHGACVSSHNCAMVCRDEGFSGGRCRGFRRRCFCTKLC

SEQ ID NO: 201 - protein, 50 aa, Gossypium hirsutum
DGDKICESPSNAFKGLCLRDDNCDIVCKTEGFPNGDCKGFLRKCVCTKPC

SEQ ID NO: 202 - protein, 48 aa, Gossypium hirsutum
KTCETQSGKFKGMCMSSTNCASVCKSEPGFDGGHCQGFRRQCVCTKPC

SEQ ID NO: 203 - protein, 48 aa, Gossypium hirsutum
KTCVAESGKFKGMCMSSTNCASVCKSEPGFDGGHCQGFRRRCLCTKPC

SEQ ID NO: 204 - protein, 48 aa, Gossypium hirsutum
RTCETKSSEYKGICLFDANCDSICKVEPGFDGGHCHGFFRRCYCTKPC

SEQ ID NO: 205 - protein, 48 aa, Gossypium hirsutum
KTCVAESGKFKGMCMSSTNCASVCKSEPDFDGGHCQGFRRRCLCTKPC

SEQ ID NO: 206 - protein, 48 aa, Gossypium hirsutum
RTCETKSSEYKGICLFDANCDSICKIESGFDGGHCHGFFRRCYCTKPC

SEQ ID NO: 207 - protein, 65 aa, Gossypium hirsutum
DEEHKEIEIGTMAEEEHKICESKSGKFKGMCFLDANCDSICKAEPGSTGGHCHGIFRICYCTKPC

SEQ ID NO: 208 - protein, 48 aa, Gossypium hirsutum
RKCESPSHKFKGMCMNRDNCATVCQTEGYEDGKCEGFRRRCICIKPCF

SEQ ID NO: 209 - protein, 52 aa, Gossypium hirsutum
GMVEGRICESKSHRFKGVCLSDHNCGLVCRNEGFLDGWCRGFRHRCFCTRNC

SEQ ID NO: 210 - protein, 47 aa, Gossypium hirsutum
RVCQSQSHKFHGACLSSHNCAMVCRNEGFSGGRCRGFRRRCFCTKLC

SEQ ID NO: 211 - protein, 50 aa, Cajanus cajan
DGDKICESRSNAFKGLCLRDNNCDIVCKTEGFPNGGCKGFIRKCVCTKPC

SEQ ID NO: 212 - protein, 48 aa, Cajanus cajan
GTTCKATSNIYKGLCLLDDSCDIVCKREGFHSGNCKGFLRKCVCTNPC

SEQ ID NO: 213 - protein, 50 aa, Cajanus cajan; identical to SEQ ID NO: 201

SEQ ID NO: 214 - protein, 48 aa, Cajanus cajan
KTCEVESGKFKGMCMSSTNCASVCKSEPGFDGGHCQGFRRRCLCTKPC

SEQ ID NO: 215 - protein, 48 aa, Cajanus cajan; identical to SEQ ID NO: 214

SEQ ID NO: 216 - protein, 48 aa, Cajanus cajan
RTCETKSSEYKGICLFDANCDSICKIEPGFDGGHCHGFFRRCYCTKPC

SEQ ID NO: 217 - protein, 48 aa, Cajanus cajan; identical to SEQ ID NO: 208

SEQ ID NO: 218 - protein, 47 aa, Vitis vinifera
RVCESQSHKFEGACMGDHNCALVCRNEGFSGGKCKGLRRRCFCTKLC

SEQ ID NO: 219 - protein, 47 aa, Vitis vinifera
RTCESQSHRFKGTCVRQSNCAAVCQTEGFHGGNCRGFRRRCFCTKHC

SEQ ID NO: 220 - protein, 47 aa, Helianthus annuus
RICESPSHKFKGICLSDTNCSSVCQSEGFPWGKCIGFRRRCFCLREC

SEQ ID NO: 221 - protein, 47 aa, Helianthus annuus
RTCESQSHKFKGTCLSDTNCANVCHSERFSGGKCRGFRRRCFCTTHC

SEQ ID NO: 222 - protein, 47 aa, Helianthus annuus
RMCQSQSHMFHGTCMRDHNCALVCRNEGFSGGKCRGFRRRCFCTRLC

SEQ ID NO: 223 - protein, 47 aa, Helianthus annuus
RICESQSQEFHGSCMSNHNCGLVCRTEGFSAGICRGVRGRCFCLKTC

SEQ ID NO: 224 - protein, 47 aa, Hydrangea macrophylla
RTCESKSNRFKGPCARDSNCATVCQTEGFYGGNCKGFRRRCFCTKNC

SEQ ID NO: 225 - protein, 47 aa, Hydrangea macrophylla
RTCESKSHRFKGPCVSATNCGSVCQTEGFHGGNCRGFRRRCFCTKHC

SEQ ID NO: 226 - protein, 71 aa, Vanilla planifolia
RRCRAQSFTFKGICYSSHSCALVCKKEGYETGHCSRGFCTLLKPANAEQRAQSLQVFASVPTPALLCVSKK

SEQ ID NO: 227 - protein, 58 aa, Cicer arietinum
SEVTIRVAEGRECRDLSHKFKGFCVRDRNCANVCSTEGFTNGHCDGFRRRCFCSRPCA

SEQ ID NO: 228 - protein, 49 aa, Cajanus cajan
RVCETPSHKFQGICISSRNCASTCASEHFPGGYCRGLRRRCMCTRPCVN

SEQ ID NO: 229 - protein, 47 aa, Cajanus cajan
RTCETPSRKFRGICVSRTNCANVCKTEGFPGGRCRGFRRRCFCLEHC

SEQ ID NO: 230 - protein, 47 aa, Cajanus cajan
RICESPSYSFKGICVSRTNCANVCKTEGFPGGRCRGLRRRCFCLKHC

SEQ ID NO: 231 - protein, 47 aa, Spinacea oleracea
RTCESRSYRFKGICVSRTNCANVCKTEGFPGGRCRGVRRRCFCLKHC

SEQ ID NO: 232 - protein, 47 aa, Cajanus cajan
RTCETPSHKFKGICVSRTNCANVCKTEGFPAGRCRGFRRRCFCLTHC

SEQ ID NO: 233 - protein, 47 aa, Ipomoea triloba
RICESKSHHYKGMCLRDHNCAMVCRTEGFSGGHCIGARRRCFCTRGC

SEQ ID NO: 234 - protein, 47 aa, Lupinus albus
RTCESQSHRFKGACLSDTNCGSVCHNEGFNGGHCRGFRRRCFCTKHC

SEQ ID NO: 235 - protein, 52 aa, Lupinus albus
FLGSHRCGKVLSSRFKGKCLTDHNCDSVCQLEGYVGGDCHGVRRRCFCNKKC

SEQ ID NO: 236 - protein, 47 aa, Lupinus albus
RHCESKSHRFKGLCHSDHNCASICLVEGFTSGKCHGFRQRCFCTKVC

SEQ ID NO: 237 - protein, 49 aa, Lupinus albus
RTRVCESQSHLFKGPCSRDHNCALVCRNEAFSGGRCKGFRRRCFCTKLC

SEQ ID NO: 238 - protein, 59 aa, Lupinus albus
MVIETEGVLGRNRCDPVLSSRFKGKCFSDHNCNSVCQLEGYTDGNCHGLRQRCFCNRKC

SEQ ID NO: 239 - protein, 59 aa, Lupinus albus
SDNNVGLKRAEGRVCQSKSHRFKGPCLSYSNCGHVCRNEGFSGGRCRGLRRRCFCTKNC

SEQ ID NO: 240 - protein, 59 aa, Lupinus albus
EMGPTMMEEARTCKSKSQRYKGSCYSDNNCAIICEGEGFTGGKCHGFFCKCWCKRPCLD

SEQ ID NO: 241 - protein, 69 aa, Lupinus albus
KECSTKSDRFRGTCFRSSNCAAVCNSENFPSGTCRGFPGTCYCTSNCAINAPITNPPPEVVEPPPEVVE

SEQ ID NO: 242 - protein, 48 aa, Lupinus albus
KTCESRSHKLKGFCYSSKRCAHVCTSHHNFIGGHCRGFIRRCHCTKPC

SEQ ID NO: 243 - protein, 48 aa, Lupinus albus
NNCKYESNKFHGVCLSDTNCESVCQTEGFPFGKCDGVLPRTCVCKKPC

SEQ ID NO: 244 - protein, 47 aa, Cajanus cajan
RHCESKSHRFHGLCLSHHNCASVCHLEGFTGGKCRGFRKRCFCKKRC

SEQ ID NO: 245 - protein, 48 aa, Cajanus cajan
RVCQSQSHRFRGGCWGDHNCALVCRNEGFSGGRCRGFRRRCFCTRLCN

SEQ ID NO: 246 - protein, 47 aa, Cajanus cajan
RVCETKSHTFHHSCVSGHNCAQVCKTEGFTGGKCRGFRRRCFCTKRC

SEQ ID NO: 247 - protein, 47 aa, Cajanus cajan
RDCESQSHRFKGPCVSDTNCASVCHGEKFSGGHCRGFRRRCFCTKHC

SEQ ID NO: 248 - protein, 55 aa, Lactuca sativa
MGVETKSCRYSSKTYKGLCFYSRNCDIICKAEGAPGGGHCQILTLRCFCNVCDPD

SEQ ID NO: 249 - protein, 47 aa, Manihot esculenta
RICESQSHRFKGVCVRKSNCAAICQTEGFHGGHCRGLRRRCFCTKHC

SEQ ID NO: 250 - protein, 47 aa, Manihot esculenta
RVCESKSHHFRGLCTIAHNCAMVCRNEGFSGGKCRGFRRRCFCTRLC

SEQ ID NO: 251 - protein, 47 aa, Manihot esculenta
RVCESKSHHFRGVCTIAHNCAIVCRNEGFSGGKCKGFRRRCFCTRLC

SEQ ID NO: 252 - protein, 47 aa, Manihot esculenta
RKCESQSHKFKGICLSDTNCASVCKTEGFTGGDCKGARRRCFCTRQC

SEQ ID NO: 253 - protein, 47 aa, Manihot esculenta
RKCESQSHRFKGMCTRHSNCAAICQTEGFHAGHCRGLRRRCFCTKHC

SEQ ID NO: 254 - protein, 47 aa, Malus domestica
RVCQSQSHGFHGTCLRHHNCALVCRNEGFSGGRCRGLRRRCFCTRLC

SEQ ID NO: 255 - protein, 57 aa, Malus domestica
RTESSKAVEGKICEFPSTLFKGLCFSSNNCKHTCRKEQFTRGKCSLLTRTCMCTKKC

SEQ ID NO: 256 - protein, 47 aa, Malus domestica
RTCESQSNRFKGTCVSKSNCAAVCQTEGFPGGNCRGLRRRCFCTKHC

SEQ ID NO: 257 - protein, 47 aa, Malus domestica
RTCESHSRKYKGTCLGKSNCASVCQTEGFHGGHCRGFRRRCFCTKHC

SEQ ID NO: 258 - protein, 63 aa, Malus domestica
RVCEVPSKTFRGLCFSDTHCIVKCHSEGFGYGKCSHVLRQCRCLKACGRLAESETTHGTETSP

SEQ ID NO: 259 - protein, 57 aa, Malus domestica
RTEPSKAVEGKICEVPSTLFKGLCISSNNCKHTCRKEQFTRGHCSVFTRACVCTKKC

SEQ ID NO: 260 - protein, 57 aa, Malus domestica
RVESSKAVEGKICEVPCTLFKGLCFSSNNCKHTCRKEQFTRGHCSVFTRACVCTKKC

SEQ ID NO: 261 - protein, 57 aa, Malus domestica
RTESNKAVEGKICEYPSTLFKGLCFSSNNCKHTCRKEQFTRGHCSVFTRACVCTKKC

SEQ ID NO: 262 - protein, 60 aa, Malus domestica
KTNENSSKNEKSSKICESPSHKYRGICFRSSNCATTCKKEGFMGGKCAGFRMRCVCTKKC

SEQ ID NO: 263 - protein, 60 aa, Malus domestica
KTKEESSKNEKSSKICESPSHKYRGICFRSSNCATTCKKEGFMGGKCAGFRMRCVCTKKC

SEQ ID NO: 264 - protein, 59 aa, Malus domestica
KTESSKAVEGKICEVPSTLFKGLCISSNNCKHTCRKEQFTRGKCIGDGFTRKCVCTKKC

SEQ ID NO: 265 - protein, 57 aa, Malus domestica
RSKSSKEVQERTCEAASGKFKGLCFSSTNCKNTCKREQFTGGKCRGFRRRCMCNKKC

SEQ ID NO: 266 - protein, 57 aa, Malus domestica
RSKSDKVAKERTCEAASGKFKGLCFSSTNCKNTCKVEKFTGGQCQGFRRRCMCNKKC

SEQ ID NO: 267 - protein, 57 aa, Malus domestica
RSKSGKAVKERTCEATSGKFKGMCFSSTNCKNTCKTEKFIGGECHGFRRRCICNKKC

SEQ ID NO: 268 - protein, 57 aa, Malus domestica
RTESSKAIEGKICEVPSTLFKGLCFSSNNCKHTCRKEQFTRGKCSGLTRTCVCTKKC

SEQ ID NO: 269 - protein, 57 aa, Malus domestica
RTESSKAVEGKICEVPSTLFKGLCFSSNNCKHTCRKEQFTRGKCSGLTRTCMCTKKC

SEQ ID NO: 270 - protein, 57 aa, Malus domestica
MTESNKAVEGKICEVPSTLFKGLCFSSNNCKHTCRKEQFTRGKCSGLTRTCMCTKKC

SEQ ID NO: 271 - protein, 60 aa, Malus domestica
VEARSKSSKVVEERTCEAASGKFKGLCFSSNNCKHTCKREQFTGGKCRGFRRRCMCNKKC

SEQ ID NO: 272 - protein, 47 aa, Malus domestica
RTCDSQSHRFKGSCVSKSNCATVCQTEGFRGGHCRGFRRRCFCTKHC

SEQ ID NO: 273 - protein, 60 aa, Malus domestica
KTKEKSSKNEKSSKTCESPSHKYHGICFRSSNCATTCKKEGFMGGKCTGFWMRCVCTKKC

SEQ ID NO: 274 - protein, 60 aa, Malus domestica
KTKEKSSKNEKSSKICESPSHKFHGICFRSSNCATTCKKEGFMGGKCTGFRMRCVCTKKC

SEQ ID NO: 275 - protein, 61 aa, Malus domestica
KIRHMSKDENSLTCESRSHKYHGPCFRFIFSSNCAVTCKGEGFTGGKCKGFHMRCICTKQC

SEQ ID NO: 276 - protein, 59 aa, Malus domestica
KTKGKPSKNVKLSKTCESPSQKYPGICRSSYCAAICKQQGLMGGKCTGFRRKCVCTKKC

SEQ ID NO: 277 - protein, 57 aa, Malus domestica
RTESSKAIEGKICEFPSTLFKGLCFSSNNCKHTCRKEQFTRGKCSLLTRTCMCTKKC

SEQ ID NO: 278 - protein, 60 aa, Malus domestica
RSGMTSKGAAEKKKCDSHPSSKFKGVCFFTNNCVDTCKLEGSSGGQCRGFRRRCICTKQC

SEQ ID NO: 279 - protein, 57 aa, Malus domestica
KSKSSKEVEKRTCEAASGKFKGMCFSSNNCANTCAREKFDGGKCKGFRRRCMCTKKC

SEQ ID NO: 280 - protein, 47 aa, Malus domestica
RTCESQSNRFKGSCVSKSNCAAVCQTEGFPGGNCRGLRRRCFCTKHC

SEQ ID NO: 281 - protein, 47 aa, Malus domestica
RTCESQSRKFKGTCLSKTNCASVCQTEGFPGGNCRGFRRRCFCTKHC

SEQ ID NO: 282 - protein, 57 aa, Medicago truncatula
QPTHVEEPEARTCDSQSHSFKGVCWIKHNCANVCKTEGFTGGHCHGFRRRCFCSKPC

SEQ ID NO: 283 - protein, 76 aa, Medicago truncatula
EEFNFAPFTPLHFRVESCFTRSRRYNGICLSDESCFKACSLEGYACGRCEKRRPYLGDHCCCKKSCQLKLVLAVLC

SEQ ID NO: 284 - protein, 50 aa, Medicago truncatula
RKHCREKSRLFEELCFNSEDCANTCRYEGFHLGGKCWGLFRTCYCKKKCR

SEQ ID NO: 285 - protein, 47 aa, Medicago truncatula
RHCESKSHRFKGMCMSDHNCASVCHVEGFPGGNCRGFRRRCFCKKRC

SEQ ID NO: 286 - protein, 47 aa, Medicago truncatula
RTCESQSHKFKGPCASDHNCASVCQTERFSGGHCRGFRRRCFCTTHC

SEQ ID NO: 287 - protein, 47 aa, Medicago truncatula
RTCETPSNSFKGACFSDTNCASVCQTEGFPGGHCKGFRQRCFCTKPC

SEQ ID NO: 288 - protein, 47 aa, Medicago truncatula
RTCETPSNNFKGLCVSDTNCASVCQTEGFPGGHCEGFRQRCFCTKPC

SEQ ID NO: 289 - protein, 50 aa, Medicago truncatula
RKCLSQSHSFKGLCLSDQNCATVCLTEGFTDGRCRGFRQRCFCSKPCLKV

SEQ ID NO: 290 - protein, 47 aa, Medicago truncatula
RRCESKSHKFKGPCSRDSNCASVCRGEGFTGGDCRGFRRRCFCTRNC

SEQ ID NO: 291 - protein, 47 aa, Medicago truncatula
RRCESQSHKFKGPCVSDSNCGSVCRGEGFIGGDCRGVRHRCFCTRNC

SEQ ID NO: 292 - protein, 48 aa, Cucumis melo
RVCESPSHNFKGLCFSDTNCGNICKTEGFSGGLCRGFRRRCFCTKHCV

SEQ ID NO: 293 - protein, 47 aa, Cucumis melo
RMCQSQSHHFHGTCIRDHNCALVCRTEGFSGGECVGFRRRCFCTHRC

SEQ ID NO: 294 - protein, 58 aa, Mimulus guttatus
VKEVGTVEDEKKVCKYKSKKFWGVCLSDIRCTDVCKREGFQGGDCQGIRRRCLCYNRC

SEQ ID NO: 295 - protein, 48 aa, Mimulus guttatus
TRTCEAKSHRFKGICARKANCAAVCQTEGFHGGHCRGFRRRCFCTKHC

SEQ ID NO: 296 - protein, 47 aa, Mimulus guttatus
RVCESKSASYKGVCVLDVTCKLACLNEGFADGDCEGVRRRCMCRRPC

SEQ ID NO: 297 - protein, 39 aa, Mimulus guttatus
FKGICVLDETCRLACLNEGFSDGDCEGLRRRCLCRKPCK

SEQ ID NO: 298 - protein, 72 aa, Mimulus guttatus
ALCETKSSIFTGACFSDRNCETICEKEGFLNGRCKLWKCICSKDCGIGGGGGVVVPPGGGGDVPPGGGDDVP

SEQ ID NO: 299 - protein, 48 aa, Mimulus guttatus
RMCETQSHGFKGQCWSEHNCRLVCAHEGFRGGKCRGFRKRCFCTRPCH

SEQ ID NO: 300 - protein, 47 aa, Mimulus guttatus
RTCLSQSQRFRGPCVSDTNCRLVCHNEGFSGGDCQGFRRRCFCTKHC

SEQ ID NO: 301 - protein, 64 aa, Mimulus guttatus
QSNVDGEIADYLKIEEKKRCNYESKKFWGICLSDSKCGEVCTKEGFEGGDCKGLRRRCYCFNNC

SEQ ID NO: 302 - protein, 71 aa, Mimulus guttatus
SLCETPSRYFNGICFSRRNCGAICEKEGFSSGRCKAFKCVCGKDCNAAGGGGGGGGGDPGLGPPVEGPPDQ

SEQ ID NO: 303 - protein, 47 aa, Mimulus guttatus
RTCESQSHGFKGRCLSDHNCGMVCKNEGFSNGWCRGFRQRCFCTRAC

SEQ ID NO: 304 - protein, 47 aa, Mimulus guttatus
RTCTSKSSNFKGLCFSSHNCKAVCNGENFPDGKCHGFKRRCFCQKPC

SEQ ID NO: 305 - protein, 47 aa, Mimulus guttatus
RTCESQSFNFRGPCVIDHNCKAVCESEGFTDGDCQGFRRRCFCRKPC

SEQ ID NO: 306 - protein, 62 aa, Cajanus cajan
ALCEKPSRLYNGSCTEERLCIEICQKEAYPSGRCDLLKCMCINKCPPVGAGQGAGSDGQRGI

SEQ ID NO: 307 - protein, 47 aa, Nicotiana tabacum
RTCESQSHRFKGPCSRDSNCATVCLTEGFSGGDCRGFRRRCFCTRPC

SEQ ID NO: 308 - protein, 47 aa, Nicotiana tabacum
RHCESQSQRFRGLCVREKNCAAVCETEGFSGGDCRGLRRRCFCTRPC

SEQ ID NO: 309 - protein, 47 aa, Nicotiana tabacum
RVCMSPSHGYHGPCWHDHNCALVCRNEGFSGGDCVGIQRKCYCTKLC

SEQ ID NO: 310 - protein, 47 aa, Nicotiana tabacum
RTCESQSHRFHGTCVRGSNCASVCQTEGFIGGNCRGLRRRCFCTRNC

SEQ ID NO: 311 - protein, 58 aa, Nicotiana tabacum
TEMGPMTIAEARRCESKSQRFRGPCVREKNCAAVCETEGFSGGDCRGLRRRCFCTRPC

SEQ ID NO: 312 - protein, 48 aa, Nelumbo nucifera
RTCESQSHRFKGICVRKSNCAAVCQTEGFHGGHCRGFRRRCFCTKHCA

SEQ ID NO: 313 - protein, 13 aa, Nelumbo nucifera
CRDGAKSSRGKDL

SEQ ID NO: 314 - protein, 49 aa, Nelumbo nucifera
KMCKTPSRQFRGLCLIGRNCDNVCRGEGFPDGDCDGFRRRCICSRPCRP

SEQ ID NO: 315 - protein, 100 aa, Nelumbo nucifera
RMCDTPSQKFVGLCLIDRNCENVCKTEGFPAGHCEGLRRRCFCSEPCPPNNGMERKFLCGVLLFLTLLYASLGEEMVVNEGPADGGRVCSTPSLNYRGPC

SEQ ID NO: 316 - protein, 49 aa, Nelumbo nucifera
RMCDTPSRLFRGLCLINRNCDNTCRVEGFPDGHCKGLRRRCFCSRPCRP

SEQ ID NO: 317 - protein, 49 aa, Cajanus cajan
RICESLSHRFKGPCVRNANCAAVCQTEGFPGGHCRGFRRRCFCTKPCSD

SEQ ID NO: 318 - protein, 47 aa, Cajanus cajan
RICESLSHRFKGPCVRRGNCAAVCQTEGFPGGLCRGFRRRCFCTKHC

SEQ ID NO: 319 - protein, 70 aa, Oryza sativa
IICKARSKMYRGKCRGNRNCAMICVHEEYTGGYCSKGVFSKCMCTKRCGGGGGGGGGGGGGGGDEPPLRE

SEQ ID NO: 320 - protein, 70 aa, Oryza sativa
ICHDHSQTFKGMCFHTSNCIACCTNEGYTGGYCKPFTYRCMCTKDCGGDSPPDDPPPAMPTSPAATTTVA

SEQ ID NO: 321 - protein, 59 aa, Oryza sativa
RLCEKPSSHFKGLCLRSQNCDNECMLEGYMDGKCKYLTRRCICSMECATMSNVGLTPTK

SEQ ID NO: 322 - protein, 49 aa, Oryza sativa
RHCLSQSHRFKGMCVSSNNCANVCRTESFPDGECKSHGLERKCFCKKVC

SEQ ID NO: 323 - protein, 48 aa, Oryza sativa
RTCESQSHRFKGPCARKANCASVCNTEGFPDGYCHGVRRRCMCTKPCP

SEQ ID NO: 324 - protein, 49 aa, Oryza sativa
RHCVSQSHRFVGACMRKSNCEHVCMTEGFPWGECRFHGIERKCFCKKRC

SEQ ID NO: 325 - protein, 47 aa, Petunia axillaris
RTCESQSHRFKGPCVSKSNCASVCQTEGFHGGHCRGLRRRCFCTKHC

SEQ ID NO: 326 - protein, 49 aa, Petunia axillaris
RYRVCLSPSHRYKGMCLRDHNCALVCRNEGFSGGDCIGFRRRCFCTKLC

SEQ ID NO: 327 - protein, 49 aa, Petunia axillaris
EARICESQSHRFHGTCVRESNCASVCQTEGFIGGNCRGFRRRCFCTRNC

SEQ ID NO: 328 - protein, 47 aa, Petunia axillaris
RTCETPSQRFKGPCVSDTNCANVCQTEGFPGGDCQGLRRRCFCTKPC

SEQ ID NO: 329 - protein, 47 aa, Petunia axillaris
RTCETLSHRFRGPCVREKNCAAVCETEGFSGGDCRGLRRRCFCTRPC

SEQ ID NO: 330 - protein, 47 aa, Petunia axillaris
RVCMSPSHGYRGPCIRDHNCALVCRDEGFSGGDCVGIQRKCYCTKLC

SEQ ID NO: 331 - protein, 50 aa, Petunia axillaris
RTCEHRSQHYYGACFANNSNMNNNCDSMCKCDGFDGGFCRLTNCFCHVKC

SEQ ID NO: 332 - protein, 47 aa, Petunia axillaris
KLCQYKSKTFHGVCVTGNTCNQKCQSEAFDGGRCQGVRHRCYCYRTC

SEQ ID NO: 333 - protein, 51 aa, Petunia axillaris
RVCRTNSKRYSGPCFNNNNNRNQNCDFICRRCEGFEGGHCSSRTCVCTKSC

SEQ ID NO: 334 - protein, 47 aa, Punica granatum
RTCESQSHKFRGTCVSNTNCGSVCQTEGFHGGHCRGFRRRCFCTKQC

SEQ ID NO: 335 - protein, 47 aa, Punica granatum
RICESKSHRFRGVCMSNSNCASVCHNEGFPGGRCRGVRKRCFCTKVC

SEQ ID NO: 336 - protein, 47 aa, Solanum tuberosum
RTCESQSHRFKGPCVSEKNCASVCETEGFSGGDCRGLRRRCFCTRPC

SEQ ID NO: 337 - protein, 47 aa, Solanum tuberosum
RNCESLSHRFKGPCTRDSNCASVCETERFSGGNCHGFRRRCFCTKPC

SEQ ID NO: 338 - protein, 47 aa, Solanum tuberosum
RTCESQSRRFKGLCFSKSNCGSVCHTEGFSGGHCRGFRRRCFCTRHC

SEQ ID NO: 339 - protein, 47 aa, Solanum tuberosum
RTCESQSHKYKGPCVRKSNCGAVCQTEGFTGGHCRGVRRRCFCTKHC

SEQ ID NO: 340 - protein, 47 aa, Solanum tuberosum
RTCESQSHSFKGPCVGDTNCASVCQTEGFIGGDCRGLRRQCFCTRNC

SEQ ID NO: 341 - protein, 47 aa, Solanum tuberosum
RTCESQSNRFKGTCVRDSNCATVCQTEGFIGGNCRGFRRRCFCTRNC

SEQ ID NO: 342 - protein, 49 aa, Solanum tuberosum
RVCISQSHRYKGPCLRDHNCALVCRNEYFSGGDCIGFGFHRKCYCTKFC

SEQ ID NO: 343 - protein, 46 aa, Phaseolus vulgaris
KTCFTKKEGWGRCIADFTCARSCRNVGYIGGKCQGLTRRCFCVLNC

SEQ ID NO: 344 - protein, 50 aa, Phaseolus vulgaris
AEGKICRTKSRKYTGACYSNDICANYCKGDGFDDGDCGGLSHICFCTKNC

SEQ ID NO: 345 - protein, 47 aa, Phaseolus vulgaris
RLCESPSHSFKGLCFNDRNCANVCLTEGFTGGKCEGARHRCFCTIIC

SEQ ID NO: 346 - protein, 47 aa, Phaseolus vulgaris
RLCWSESQAFKGLCFSNDNCATVCLTEGFTGGKCEGLRRRCFCSKNC

SEQ ID NO: 347 - protein, 47 aa, Phaseolus vulgaris
RGCESRSRHLRGKCESDENCASACRTERFSGGHCRGFRCRCFCDKRS

SEQ ID NO: 348 - protein, 47 aa, Phaseolus vulgaris
RTCESQSHRFKGPCVSDTNCASVCRTERFSGGHCRGFRRRCFCTKHC

SEQ ID NO: 349 - protein, 47 aa, Phaseolus vulgaris
RVCESQSHGFKGACTGDHNCALVCRNEGFSGGNCRGFRRRCFCTKIC

SEQ ID NO: 350 - protein, 47 aa, Phaseolus vulgaris
RHCESKSHRFKGMCLSDRNCASVCHLEGFTGGNCRGFRRRCFCTRHC

SEQ ID NO: 351 - protein, 47 aa, Cajanus cajan
RVCLSQSHHYKGPCLRGHNCAMVCKTEGFAGGECKGFISRCFCAKLC

SEQ ID NO: 352 - protein, 47 aa, Cajanus cajan
RVCLSQSHSFKGPCVRGHNCASVCKTEGFPGGECKGFRRRCFCAKPC

SEQ ID NO: 353 - protein, 47 aa, Cajanus cajan; identical to SEQ ID NO: 352

SEQ ID NO: 354 - protein, 47 aa, Cajanus cajan
RVCQSQSHYFKGPCARDHNCAWVCRNEGFSGGRCKGFRRRCFCTKLC

SEQ ID NO: 355 - protein, 47 aa, Cajanus cajan; identical to SEQ ID NO: 354

SEQ ID NO: 356 - protein, 47 aa, Prunus persica
RVCQSQSHGFRGPCIRHHNCALVCRNEGFSGGRCRGFRRRCFCTRLC

SEQ ID NO: 357 - protein, 47 aa, Prunus persica
RTCESQSNRFKGTCVSTSNCASVCQTEGFPGGHCRGFRRRCFCTKHC

SEQ ID NO: 358 - protein, 57 aa, Prunus persica
RDAKKVEPKKKTCDSQSQKFKGMCFLTSSCVQSCKSEGFTGGQCRGFRRRCFCSKNC

SEQ ID NO: 359 - protein, 57 aa, Prunus persica
RSAKKVEPKKRTCESQSQKFKGICFLSSNCATSCTTEGFNGGQCRGFRRRCFCSKAC

SEQ ID NO: 360 - protein, 57 aa, Prunus persica
KICEVTSKTFIGLCIKDRNCMVRCHSEGYGYGKCSHILRKCRCLKPCVAEETKENLP

SEQ ID NO: 361 - protein, 55 aa, Prunus persica
KVATKEESRTCESLSTKFKGFCLRSSRCAEACQKEGFMGGKCPGFRLRCTCTKKC

SEQ ID NO: 362 - protein, 55 aa, Prunus persica; identical to SEQ ID NO: 361

SEQ ID NO: 363 - protein, 55 aa, Prunus persica
KVETKETSRTCESLSTKFKGPCIRSSNCANICEEEGFKGGKCVGFRLRCTCTKNC

SEQ ID NO: 364 - protein, 55 aa, Prunus persica; identical to SEQ ID NO: 363

SEQ ID NO: 365 - protein, 55 aa, Prunus persica; identical to SEQ ID NO: 363

SEQ ID NO: 366 - protein, 55 aa, Prunus persica; identical to SEQ ID NO: 363

SEQ ID NO: 367 - protein, 55 aa, Prunus persica
KVETKETSRTCESLSTKFKGPCFRSSNCANICEKEGFKGGKCVGFRLRCTCTKNC

SEQ ID NO: 368 - protein, 55 aa, Prunus persica
KAATKENSRTCESLSTKFKGLCFRSSNCADICKKEGFLEGKCAGFRLRCTCTKKC

SEQ ID NO: 369 - protein, 47 aa, Pisum sativum
RDCESKSHKFKGTCLSDTNCASVCQTERFTGGHCRGFRHRCYCTTHC

SEQ ID NO: 370 - protein, 47 aa, Pisum sativum
RDCESPSQKFKGLCFSDTNCASVCQTEGFPGGKCEGLRHRCFCTKPC

SEQ ID NO: 371 - protein, 47 aa, Pisum sativum
RDCESPSQKFKGVCLSDRNCASVCQTEGFTGGDCKGLRQRCFCTKPC

SEQ ID NO: 372 - protein, 47 aa, Pisum sativum
RDCESPSQIFKGLCFTDTNCASVCHTEGFPGGKCKGLPKRCICTKPC

SEQ ID NO: 373 - protein, 46 aa, Pisum sativum
AGTCPSPSTKFKGVCVSDFDCMITCQMEGLSEGKCQSLKCICAKPC

SEQ ID NO: 374 - protein, 39 aa, Pisum sativum
SECNDEECEWSCIDKGFLDGVCSPSSNQCFCVIHRVIIR

SEQ ID NO: 375 - protein, 50 aa, Pisum sativum
NSCQSKSSQFKGICLSNKKCARVCHLEGFLEGGKCKGKLLKHCSCKKSIC

SEQ ID NO: 376 - protein, 45 aa, Pisum sativum
RGCKVKSTKFRGPCSFDKNCATVCRGEGYRDGDCHGLRRRCMCLC

SEQ ID NO: 377 - protein, 47 aa, Pisum sativum
RKCVSQSHRFHGPCLRDSNCATVCLGEGFTGGDCHGFRRRCFCSRFC

SEQ ID NO: 378 - protein, 56 aa, Pisum sativum
RHCESPSHRFKGICVRKSNCAAICQTEGFSSGKCRGFRSRCVCIKHCHHHEEEVQN

SEQ ID NO: 379 - protein, 57 aa, Pisum sativum
RLCESASHRFKGICVRKSNCGAVCQTEGFSGGKCRGLRARCMCTKHCHQEEEAEVKN

SEQ ID NO: 380 - protein, 57 aa, Pisum sativum
MRVDGARTNRMCDSKSHKFRGLCVSDTNCRNVCQTERFSDGHCRGARRRCYCQNPCI

SEQ ID NO: 381 - protein, 57 aa, Pisum sativum; identical to SEQ ID NO: 379

SEQ ID NO: 382 - protein, 47 aa, Quercus lobata
RTCESQSHRFKGPCVRKSNCASVCQTEGFHGGQCRGFRRRCFCTKHC

SEQ ID NO: 383 - protein, 47 aa, Quercus lobata
RVCESQSHKFKGACMSNHNCALVCRNEGFSGGRCRGFRHRCFCTRIC

SEQ ID NO: 384 - protein, 50 aa, Rosa chinensis
ENLRTCDSPSGKFKGPCIISSNCALTCQKEGFAGGHCRDFLRKCYCAKPC

SEQ ID NO: 385 - protein, 47 aa, Rosa chinensis
RTCETLSHKFKGTCLRESNCASVCQTEGFSGGDCRGLRRRCFCTKHC

SEQ ID NO: 386 - protein, 59 aa, Rosa chinensis
GIGPNVMVAEARTCESQSLKYEGMCLRESHCASVCQTEGYSGGDCHGLYSICVCTKDCQ

SEQ ID NO: 387 - protein, 58 aa, Rosa chinensis
GIGPNVTVAEARKCESQSHKFAGICLIESNCASICKTEGYSGGNCGGFRRRCFCTKDC

SEQ ID NO: 388 - protein, 47 aa, Rosa chinensis
RKCESQSHKFEGICLKQSNCASICKTEGYSGGNCRGFRRRCFCTKDC

SEQ ID NO: 389 - protein, 58 aa, Rosa chinensis
GIGPNVMVAEARKCESQSHKFAGICLIESNCASICKTEGYSGGNCGGFRRRCFCTKDC

SEQ ID NO: 390 - protein, 47 aa, Rosa chinensis
RVCEVPSKTFSGLCFSDTHCIVRCHSEGYGYGKCSHILRRCRCLKAC

SEQ ID NO: 391 - protein, 48 aa, Rosa chinensis
KICEVPSKTFKGLCVKDRNCVVRCHSEGYGNGKCSHVRRQCMCLKICD

SEQ ID NO: 392 - protein, 47 aa, Rosa chinensis
KICEYPSRTFKGLCVKDKNCVVRCRSEGYNYGKCSHVRRRCRCFKPC

SEQ ID NO: 393 - protein, 47 aa, Rosa chinensis; identical to SEQ ID NO: 162

SEQ ID NO: 394 - protein, 47 aa, Rosa chinensis
RVCESRSHGFHGTCIHHNNCALVCRNEGFPGGRCRGFRRRCFCTRNC

SEQ ID NO: 395 - protein, 47 aa, Cajanus cajan
RTCESKSHRFKGICVRKANCAAVCQTEGFHSGHCRGFRRRCYCTRHC

SEQ ID NO: 396 - protein, 42 aa, Cajanus cajan
RTCESESSSFKGLCLRNADCYIVCQTEGFSGGKCSCFCTKPC

SEQ ID NO: 397 - protein, 47 aa, Cajanus cajan
RTCESESSTFKGLCLKNADCYIVCRTEGFSGGKCSSFLRRCFCTKPC

SEQ ID NO: 398 - protein, 74 aa, Cajanus cajan
VVCSAASRLFSGLCFSDSNCSSICEKEGFLTGVCKGLQCICQKDCGGGDGGDPGQGGADQSALINRKLLDQLLV

SEQ ID NO: 399 - protein, 56 aa, Cajanus cajan
AAASVVEEKRSCQSQSHKFKGKCFSDKNCGSVCQTEGFPSGKCHGLRQRCFCTRPC

SEQ ID NO: 400 - protein, 56 aa, Cajanus cajan
AMCSAPSKLFRGVCLLRSNCSSICATEGFLYGLCKDLKCVCYKDCNVVGSGGDAQH

SEQ ID NO: 401 - protein, 74 aa, Cajanus cajan
VMCSAASRLFSGLCFSDNNCSAICEKEGFLSGVCKGLKCVCQKDCNGGGGGGGNGGGDGGDQDQGPPAQGGGDQ

SEQ ID NO: 402 - protein, 74 aa, Cajanus cajan
VMCSAASRLFSGLCFSDNNCSAICEKEGFLSGVCKGLKCVCQKDCNGGGGGNGGGDGGDQDQGPPAQGGGDQSP

SEQ ID NO: 403 - protein, 73 aa, Cajanus cajan
AMCSAPSRLFRGLCFRRSNCTSICEKEGFLYGLCKGLKCVCYKDCNAAGGGGNGGDPGQGPPDQGGDDQPPPL

SEQ ID NO: 404 - protein, 56 aa, Cajanus cajan; identical to SEQ ID NO: 399

SEQ ID NO: 405 - protein, 61 aa, Cajanus cajan
AMCSAPSKLFRGVCLLRSNCSSICATEGFLYGLCKDLKCVCYKDCNVVGSGSGGSGGDAQH

SEQ ID NO: 406 - protein, 48 aa, Simmondsia chinensis
RICQSQSHLFRGPCVRNHNCASVCQNEGFSDGSCRGFRRRCFCAKFCP

SEQ ID NO: 407 - protein, 42 aa, Sechium edule
QSHGFHGACLSDHNCAVVCRNEGFSGGDCKGFRRRCFCTRHC

SEQ ID NO: 408 - protein, 48 aa, Sechium edule
RTCESPSRHFKGVCFSRNNCGHVCKTEGFHGGHCRGFRRRCFCTKHCL

SEQ ID NO: 409 - protein, 48 aa, Sechium edule
RTCESQSHHFKGLCFSKNNCGHVCKTEGFHGGHCRGFRRRCFCTKHCA

SEQ ID NO: 410 - protein, 50 aa, Sequoiadendron giganteum
RMCKSQSHTFKGYCVSDSNCKNVCRTEKFPTGGCDFHVASRKCYCYKPCA

SEQ ID NO: 411 - protein, 47 aa, Solanum lycopersicum
RTCESQSHRYKGPCVRKNNCANVCKTEGFSGGHCRGFRRRCFCAKHC

SEQ ID NO: 412 - protein, 47 aa, Solanum lycopersicum
RTCESQSHHFKGNCLSDTNCGSVCRTEGFTGGNCRGFRRRCFCTRNC

SEQ ID NO: 413 - protein, 50 aa, Solanum lycopersicum
AEARTCESQSNSFKGTCVRDSNCATVCQTEGFIGGNCRGFRRRCFCTRNC

SEQ ID NO: 414 - protein, 47 aa, Solanum lycopersicum
RTCEPQSHSFKGPCSRDSNCATVCQTEGFIGGDCRGLRRQCFCTTEC

SEQ ID NO: 415 - protein, 47 aa, Solanum lycopersicum
RTCESQSHRFKGPCVSEKNCASVCETEGFSGGDCRGFRRRCFCTRPC

SEQ ID NO: 416 - protein, 47 aa, Solanum lycopersicum
RVCMSPSHSYHGPCWHDHNCAIVCRNEGFSGGNCVGIQLKCYCTKLC

SEQ ID NO: 417 - protein, 47 aa, Solanum lycopersicum
RVCISQSHRYRGPCVLDHNCAIICRNEGFSGGDCIGWRRRCFCTKLC

SEQ ID NO: 418 - protein, 47 aa, Solanum pennellii
RTCETQSHKYKGPCVRKSNCANVCKTEGFRGGHCRGFRRRCFCTKNC

SEQ ID NO: 419 - protein, 55 aa, Solanum pennellii
GIRKTDAKLCQYHSETFHGVCVTGNNCNRHCQRENFEGGRCHGIRHYRCVCYRTC

SEQ ID NO: 420 - protein, 47 aa, Solanum pennellii
RHCESLSHRFKGPCASDKNCASICETERFSGGNCRGFRRRCFCTKPC

SEQ ID NO: 421 - protein, 47 aa, Solanum pennellii; identical to SEQ ID NO: 415

SEQ ID NO: 422 - protein, 47 aa, Solanum pennellii
RTCESQSHSFKGPCSRDSNCATVCQTEGFIGGDCRGLRRQCFCTTDC

SEQ ID NO: 423 - protein, 47 aa, Solanum pennellii
RTCESQSNSFKGTCVRDSNCATVCQTEGFIGGNCRGFRRRCFCTRNC

SEQ ID NO: 424 - protein, 47 aa, Solanum pennellii; identical to SEQ ID NO: 412

SEQ ID NO: 425 - protein, 47 aa, Solanum pennellii; identical to SEQ ID NO: 416

SEQ ID NO: 426 - protein, 47 aa, Solanum pennellii; identical to SEQ ID NO: 417

SEQ ID NO: 427 - protein, 47 aa, Cajanus cajan
RTCESKSHRFRGVCVSDGNCGNVCHNEGFGGGKCRGFRRRCFCTRHC

SEQ ID NO: 428 - protein, 47 aa, Cajanus cajan
RTCESQSHRFKGKCLSDGNCGNVCHNEGFGGGKCRGFRRRCFCTRNC

SEQ ID NO: 429 - protein, 45 aa, Cajanus cajan
KTCESPSGKYHGVCLNAKNCASVCPSEGFTGGHCKSLRCYCIKTC

SEQ ID NO: 430 - protein, 63 aa, Cajanus cajan
TEMGPADVEPRICETLSKVFKGMCWNQNNCAIMCREFEHFHSGHCRGFLRRCYCTKRCDGGPS

SEQ ID NO: 431 - protein, 52 aa, Tarenaya hassleriana
ERGEGRMCQSKSHHFHRTCVSNHNCAMVCRTEGFSGGHCVGFRRRCFCTRLC

SEQ ID NO: 432 - protein, 47 aa, Tarenaya hassleriana
RTCESPSHRFKGACLSETNCANVCHNEGFIGGNCRGLRRRCFCTKHC

SEQ ID NO: 433 - protein, 47 aa, Cajanus cajan
RTCQSQSHRFKGMCMRKSNCAAVCQTEGFHSGHCRGFHRRCFCTKHC

SEQ ID NO: 434 - protein, 47 aa, Cajanus cajan
RTCESKSHAFKGPCLSDHNCGLVCRNEGFPDGWCRGIRRRCFCTRRC

SEQ ID NO: 435 - protein, 47 aa, Cajanus cajan
RLCESKSHGFKGACLSDHNCGMVCSNEGFSGGRCRGFRHRCFCTRRC

SEQ ID NO: 436 - protein, 47 aa, Cajanus cajan
RVCESKSHKFKGPCLGDHNCALVCRNEGFSGGRCRGFRHRCFCTKLC

SEQ ID NO: 437 - protein, 47 aa, Thlaspi arvense
RTCESKSHRFKGACLSETNCKNVCHNEGFRGGNCRGLRRRCFCTRNC

SEQ ID NO: 438 - protein, 47 aa, Eutrema salsugineum
RKCESKSHRFKGKCLSETNCKNVCHNEGFTGGNCRGFRRRCFCTRNC

SEQ ID NO: 439 - protein, 46 aa, Eutrema salsugineum
RTCESASGKFQGVCLNSKSCASVCQSNEGFSGGHCNSLRCYCTKTC

SEQ ID NO: 440 - protein, 47 aa, Eutrema salsugineum
RMCQSKSHHFKHMCWSDHNCAIICRNEGFSGGRCRGFHRRCYCTRLC

SEQ ID NO: 441 - protein, 50 aa, Eutrema salsugineum
QQICKARSINFTALCLRWSKCKQACLTENFPDGRCIARRCFCMTPCHFLT

SEQ ID NO: 442 - protein, 52 aa, Eutrema salsugineum
QQICEARSINFRGMCMKWRNCQRVCMSEGFPDGRCAGFIRRCICRKPCPILS

SEQ ID NO: 443 - protein, 100 aa, Eutrema salsugineum
RMCESSSQLFNGPCLSTTNCANICHNEGFTDGDCQGFRRRCICTRPCTPRGMERKTLGLFFMLFLLLSADKYYFMDADVALKTAEARRCESKSHKFKGPC

SEQ ID NO: 444 - protein, 48 aa, Trifolium pratense
RTCESQSHKFKGPCASDHNCASVCQTERFPGGHCRGFRRRCFCTTHCS

SEQ ID NO: 445 - protein, 55 aa, Trifolium pratense
EMVVETEGRHCESKSHRFKGMCLSDHNCASVCHVEGFPGGNCRGFRRRCFCKKRC

SEQ ID NO: 446 - protein, 48 aa, Trifolium pratense
RKCESKRRWIYGNCLSDEKCASDCLVKGFLFGKCRGYDNRCYCKKPRC

SEQ ID NO: 447 - protein, 47 aa, Cajanus cajan
RVCASKSHYYKGPCLHNHNCAMVCRNEGFSGGKCHGLGRRCFCTKLC

SEQ ID NO: 448 - protein, 47 aa, Cajanus cajan
RVCLSPSHKFHGTCVRGHNCAEICRTEGFSGGDCHGFRRRCMCSRRC

SEQ ID NO: 449 - protein, 57 aa, Cajanus cajan
AGEEAVGEEKKACKYKSEKFWGICITDSKCIEVCRREGFQTGDCKGLRRRCYCVNNC

SEQ ID NO: 450 - protein, 82 aa, Vigna mungo
VASGGKDKTKKPGRYIATELENIETNYFSDYTGFEIQIDSTDVDDAIDTLSEVASKKLVQILLRFVALFLPVAPIPPLPIPS

SEQ ID NO: 451 - protein, 48 aa, Zea mays
RTCQSQSHRFRGPCLRRSNCANVCRTEGFPGGRCRGFRRRCFCTTHCH

SEQ ID NO: 452 - protein, 55 aa, Zea mays
RVVVEETLCLSQSHAFKGVCLSNTNCDNVCKTEKFTGGECKMDGVMRKCYCKKVC

SEQ ID NO: 453 - protein, 49 aa, Zea mays
RHCLSQSHRFKGLCMSSNNCANVCQTENFPGGECKAEGATRKCFCKKIC

SEQ ID NO: 454 - protein, 55 aa, Zea mays
GPVTMVSARKCESQSFRFKGPCSRDANCANVCLTEGFTGGVCKGLRHRCFCTRDC

SEQ ID NO: 455-458 - skipped (4 entries)

SEQ ID NO: 459 - protein, 16 aa, synthetic construct
GHCRGFRRRCFCTTHC

SEQ ID NO: 460 - protein, 50 aa, Medicago truncatula
KLCQKRSTTWSGPCLNTGNCKRQCINVEHATFGACHRQGFGFACFCYKKC

SEQ ID NO: 461 - protein, 18 aa, synthetic construct
GACHRQGFGFACFCYKKC

SEQ ID NO: 462 - protein, 50 aa, Medicago truncatula
KLCERRSKTWSGPCLISGNCKRQCINVEHATSGACHRQGIGFACFCKKKC

SEQ ID NO: 463 - protein, 18 aa, synthetic construct
GACHRQGIGFACFCKKKC

SEQ ID NO: 464 - protein, 53 aa, Cajanus cajan
KPCTKLSKGWRGLCAPHKCSSYCIHHEGAYHGACLKNRHSKHYGCYCYYRHCY

SEQ ID NO: 465 - protein, 22 aa, synthetic construct
GACLKNRHSKHYGCYCYYRHCY

SEQ ID NO: 466 - protein, 53 aa, Cajanus cajan
KPCTKLSKGWHGLCAPHKCSNYCIHHEGAYHGACLKNHHNKHYGCYCYYRHCY

SEQ ID NO: 467 - protein, 22 aa, synthetic construct
GACLKNHHNKHYGCYCYYRHCY

SEQ ID NO: 468 - protein, 47 aa, Medicago truncatula
RVCESQSHKFKGPCARNHNCALVCQTERFSGGRCRGFRRRCFCTRPC

SEQ ID NO: 469 - protein, 17 aa, synthetic construct
GGRCRGFRRRCFCTRPC

SEQ ID NO: 470 - protein, 17 aa, synthetic construct
GGLCRGFRRRCFCTKHC

SEQ ID NO: 471 - protein, 49 aa, Sorghum bicolor
VHVCTMRNKFFHGPCMSNKNCAASCIQHRIGGGGYCSSRRQICKCTLQC

SEQ ID NO: 472 - protein, 19 aa, synthetic construct
GGGGYCSSRRQICKCTLQC

SEQ ID NO: 473 - protein, 6 aa, synthetic construct
KREAEA

(19) United States
(12) Patent Application Publication
(10) Pub. No.: US 2026/0297145 A1
(43) Pub. Date: Oct. 1, 2026

(54) ANTIMICROBIAL DEFENSIN PEPTIDE FOLDING VARIANTS

(71) Applicant: Donald Danforth Plant Science Center, St. Louis, MO (US)

(72) Inventor: Dilip M. Shah, Chesterfield, MO (US)

(21) Appl. No.: 19/477,212

(22) PCT Filed: Apr. 23, 2024
(86) PCT No.: PCT/US2024/025770

§ 371 Date: Oct. 21, 2025

Related U.S. Application Data
(60) Provisional application No. 63/498,050, filed on Apr. 25, 2023.

(51) Int. Cl. C07K 14/47; A01N 37/46; A01P 3/00; A61K 38/00; B01D 15/34; C12N 1/06; C12N 1/16; C12N 9/76; C12N 15/81; C12P 21/02; C12R 1/84

(52) U.S. Cl. CPC C07K 14/4723; A01N 37/46; A01P 3/00; B01D 15/34; C12N 1/06; C12N 1/16; C12N 9/6427; C12N 15/815; C12P 21/02; A61K 38/00; C12R 2001/84; C12Y 304/21004

(57) ABSTRACT

Provided are compositions and methods employing Defensin peptides and proteins, including folding variants of antimicrobial Defensin peptides and proteins (DEF_PFV) that exhibit favorable antifungal properties and a desirable resistance or susceptibility to protease cleavage, which antimicrobial DEF_PFV can be applied ex vivo directly to a plant, human, or animal or can be administered in vivo to a plant, human, or animal.

### CROSS-REFERENCE TO RELATED APPLICATIONS

This application claims priority under 35 U.S.C. § 119 to provisional patent application U.S. Ser. No. 63/498,050, filed Apr. 25, 2023. The provisional patent application is herein incorporated by reference in its entirety, including without limitation, the specification, claims, and abstract, as well as any figures, tables, appendices, or drawings thereof.

### SEQUENCE LISTING

The instant application contains a Sequence Listing which has been submitted electronically in XML format and is herein incorporated by reference in its entirety. Said XML copy, created on Apr. 19, 2024, is named “P13859WO01_SequenceListing.xml” and is 426,679 bytes in size.

### TECHNICAL FIELD

The present disclosure relates, generally, to antimicrobial peptides and proteins for use in controlling pathogenic microbes. Provided herein are compositions and methods employing Defensin peptides and proteins, including folding variants of antimicrobial Defensin peptides and proteins (DEF_PFV) that exhibit favorable antifungal properties and a desirable resistance or susceptibility to protease cleavage, which antimicrobial DEF_PFV can be applied ex vivo directly to a plant, human, or animal or can be administered in vivo to a plant, human, or animal.

### BACKGROUND OF THE DISCLOSURE

Animals and plants are both susceptible to microbial infections that, if left untreated, can result in substantial morbidity and mortality in human and veterinary patients and loss of food crops, which are particularly susceptible to contamination and spoilage during storage. Protection of agriculturally important crops from pathogenic microbes is crucial in improving crop yields. Fungal infections are a particular problem in damp climates and can become a major concern during crop storage, where such infections can result in spoilage and contamination of food or feed products with fungal toxins. Unfortunately, modern growing methods, harvesting and storage systems can promote plant pathogen infections.

The control of pathogens in humans, veterinary animals, and plants is complicated by the need for simultaneous control of multiple pathogenic microbes of distinct genera, including fungi, dimorphic fungi, yeast, molds, and oomycetes. For example, plants are susceptible to infection by a wide range of microbes including Alternaria, Ascochyta, Botrytis, Cercospora, Colletotrichum, Diplodia, Erysiphe, Fusarium, Gaeumanomyces, Helminthosporium, Macrophomina, Magnaporthe, Nectria, Peronospora, Phoma, Phakopsora, Phymatotrichum, Phytophthora, Plasmopara, Podosphaera, Puccinia, Pythium, Pyrenophora, Pyricularia, Rhizoctonia, Sclerotium, Sclerotinia, Septoria, Thielaviopsis, Uncinula, Venturia, and Verticillium species are all recognized plant pathogens.

Consequently, antimicrobial agents that control a limited subset of microbial pathogens provide inadequate or ineffective protection under conditions where multiple pathogens are present. Moreover, in many cases, plant pathogenic microbes have become resistant to existing antimicrobial agents.

Antimicrobial peptides have been described for the treatment of microbial infections in both animals and plants and are reviewed in Hancock, AAC 43 (6): 1317 (1999) and Yount, PNAS 101 (19): 7363 (2004). Antimicrobial peptides include nodule-specific cysteine-rich (NCR) peptides and Defensin peptides.

Defensins are small cysteine-rich peptides of about 45-54 amino acids that constitute an important component of the innate immunity of plants (Sathoff, Phytopathology 109:402 (2019)). Defensins are widely distributed in plants and vary greatly in their amino acid sequences yet they all have a compact shape that is stabilized by either four or five intramolecular disulfide bonds. Plant Defensins have been characterized as comprising a conserved γ-core motif comprising a conserved GXCX3-9C (where X is any amino acid) sequence (Lacerda et al., Frontiers in Microbio. (2014) 5 (116): 1-10). The three-dimensional structure of the previously characterized γ-core motif consists of two antiparallel β-sheets, with an interpolated turn region (Ibid.). Antimicrobial activity of certain Defensins has been correlated with the presence of positively charged amino acid residues in the γ-core motif (Spelbrink et al., Plant Physiol., 2004, 135, 2055-2067; Sagaram et al., 2013, PLoS ONE, 8 (12): e82485).

Certain NCR peptides exhibit antimicrobial properties when applied to free-living bacteria and can mediate bacterial cell death and early nodule senescence. Yang, PNAS 114:6848-6853 (2017) and Wang, PNAS 114:6854-6859 (2017). Antimicrobial NCRs (AMPs) are cationic and have conserved cysteine residues that form intramolecular disulfide bonds. Cysteine substitutions or disulfide bond modifications can influence the antimicrobial activity of certain NCR peptides. For example, substitution of cysteines for serines, altering the cysteine pairs that form disulfide bonds, and oxidation can affect an NCR's antimicrobial activity. Haag, J. Biol. Chem. 287 (14): 10791-8 (2012) and Isozumi, Nature Sci. Rep. 11:9923 (2021).

Despite recent advancements in the control and treatment of microbial infections and the recognition that certain antimicrobial peptides exhibit substantial antimicrobial properties and activities, there remains a substantial unmet need for therapeutically effective modalities for the prevention, treatment, and control of pathogenic microorganisms.

### SUMMARY OF THE DISCLOSURE

Certain cationic antimicrobial Defensin (DEF) peptides can form alternative disulfide bonding pairs and, consequently, can fold into multiple distinct conformations each having a unique secondary (e.g., alpha helices and beta sheets) and tertiary structure, and which Defensin peptide and protein structural variants possess improved antimicrobial activities, and, optionally, exhibiting a desired resistance or sensitivity to proteolytic cleavage in comparison to other NCR PFV with different disulfide bonding pairs. As disclosed herein, an Defensin peptide folding variant that exhibits a desired level of antimicrobial activity and protease resistance or sensitivity can be purified from a heterogeneous mixture containing multiple (i.e. two or more) Defensin peptide folding variants and the purified Defensin peptide folding variant can be used advantageously in the compositions and methods disclosed herein. Thus, within various aspects, the present disclosure provides-cysteine-rich Defensin peptide folding variants 1 (DEF_PFV1), compositions comprising antimicrobial DEF_PFV1, methods for making antimicrobial DEF_PFV1 compositions, methods for using such antimicrobial DEF_PFV1 compositions, and devices, including medical devices, which comprise an antimicrobial DEF_PFV1.

In certain embodiments, provided herein are compositions comprising a Defensin peptide folding variant 1 (DEF_PFV1), wherein said Defensin peptide is a cationic Defensin peptide comprising in contiguous amino acid sequence from its N-terminal end (1) a first cysteine pair motif including C₁ and C₂, (2) a first intervening sequence (IS1), (3) a second cysteine pair motif including C₃ and C₄, (4) a second intervening sequence (IS2), (5) a third cysteine pair motif including C₅ and C₆, (6) a third intervening sequence (IS3), and (7) a fourth cysteine pair motif including C₇ and C₈, wherein cysteine residues C₁, C₂, C₃, C₄, C₅, C₆, C₇, and C₈ of said DEF_PFV1 form a first set of disulfide bonds and DEF_PFV1 thereby adopts a first conformation that exhibits greater antimicrobial activity, increased protease resistance, or increased protease sensitivity as compared to a Defensin peptide folding variant 2 (DEF_PFV2) that has the same amino acid sequence as DEF_PFV1 but forms a distinct second set of disulfide bonds between cysteine residues C₁, C₂, C₃, C₄, C₅, C₆, C₇, and C₈ and adopts a distinct second conformation, and wherein said composition is substantially free of DEF_PFV2.

In related embodiments, provided herein are compositions comprising a Defensin peptide folding variant 1 (DEF_PFV1), wherein said Defensin peptide is a synthetic variant of a native Defensin peptide comprising in contiguous amino acid sequence from its N-terminal end (1) a first amino acid pair motif including X₁ and C₂, (2) a first intervening sequence (IS1), (3) a first cysteine pair motif including C₃ and C₄, (4) a second intervening sequence (IS2), (5) a second cysteine pair motif including C₅ and C₆, (6) a third intervening sequence (IS3), and (7) a second amino acid pair motif including C₇ and Y₁, wherein said amino acid X₁ and Y₁ are each independently selected from the group of amino acids other than cysteine, and wherein cysteines C₂, C₃, C₄, C₅, C₆, and C₇ of DEF_PFV1 form a first set of disulfide bonds and DEF_PFV1 thereby adopts a first conformation that exhibits greater antimicrobial activity, increased protease resistance, or increased protease sensitivity as compared to a Defensin peptide folding variant 2 (DEF_PFV2) that has the same amino acid sequence as DEF_PFV1 but forms a distinct second set of disulfide bonds between cysteines C₂, C₃, C₄, C₅, C₆, and C₇ and adopts a distinct second conformation, and wherein said composition is substantially free of DEF_PFV2.

In other related embodiments, provided herein are compositions comprising a Defensin peptide folding variant 1 (DEF_PFV1), wherein said Defensin peptide is a synthetic variant of a native Defensin peptide comprising in contiguous amino acid sequence from its N-terminal end (1) a first amino acid pair motif including X₁ and Y₁, (2) a first intervening sequence (IS1), (3) a first cysteine pair motif including C₃ and C₄, (4) a second intervening sequence (IS2), (5) a second cysteine pair motif including C₅ and C₆, (6) a third intervening sequence (IS3), and (7) a second amino acid pair motif including X₂ and Y₂, wherein said amino acids X₁, Y₁, X₂, and Y₂ are each independently selected from the group of amino acids other than cysteine, and wherein cysteines C₃, C₄, C₅, and C₆ of DEF_PFV1 form a first set of disulfide bonds and DEF_PFV1 thereby adopts a first conformation that exhibits greater antimicrobial activity, increased protease resistance, or increased protease sensitivity as compared to a Defensin peptide folding variant 2 (DEF_PFV2) that has the same amino acid sequence as DEF_PFV1 but forms a distinct second set of disulfide bonds between cysteines C₃, C₄, C₅, and C₆ and adopts a distinct second conformation, and wherein said composition is substantially free of DEF_PFV2.

In further related embodiments, provided herein are compositions comprising a Defensin peptide folding variant 1 (DEF_PFV1) wherein said Defensin is a cationic Defensin peptide comprising a Defensin gamma core peptide sequence GXC₅X3-9C₆ (SEQ ID NO: 455) or variant gamma core peptide sequence GXC₅X3-10C₆ (SEQ ID NO: 456) and at least two additional cysteine residues selected from the group consisting of cysteine residues corresponding to C₁, C₂, C₃, C₄, C₇, and C₈ of a reference Defensin peptide, wherein said cysteine residues of DEF_PFV1 form a first set of disulfide bonds and DEF_PFV1 thereby adopts a first conformation that exhibits greater antimicrobial activity, increased protease resistance, or increased protease sensitivity as compared to a Defensin peptide folding variant 2 (DEF_PFV2) that has the same amino acid sequence as DEF_PFV1 but forms a distinct second set of disulfide bonds between cysteine residues and adopts a distinct second conformation, and wherein said composition is substantially free of DEF_PFV2. In certain embodiments, the cationic Defensin peptide comprises a Defensin gamma core peptide sequence GXC₅X3-9C₆ (SEQ ID NO: 455) or variant gamma core peptide sequence GXC₅X3-10C₆ (SEQ ID NO: 456) and cysteine residues C₇, and C₈ of a reference Defensin peptide, wherein said cysteine residues of DEF_PFV1 form a first set of disulfide bonds and DEF_PFV1 thereby adopts a first conformation that exhibits greater antimicrobial activity, increased protease resistance, or increased protease sensitivity as compared to a Defensin peptide folding variant 2 (DEF_PFV2) that has the same amino acid sequence as DEF_PFV1 but forms a distinct second set of disulfide bonds between cysteine residues and adopts a distinct second conformation, and wherein said composition is substantially free of DEF_PFV2

In other embodiments, provided herein are methods for making a composition as disclosed herein, comprising: (a) separating a fraction comprising Defensin peptide folding variant 1 (DEF_PFV1) from one or more fractions comprising Defensin peptide folding variant 2 (DEF_PFV2), or DEF_PFV2 peptide fragments thereof, from a mixture comprising DEF_PFV1 and DEF_PFV2, or DEF_PFV2 peptide fragments thereof; and (b) combining said fraction comprising DEF_PFV1, or a preparation further purified from said DEF_PFV1 fraction, with an agriculturally, pharmaceutically, or veterinary practicable carrier, diluent, or excipient, thereby making the composition.

In further embodiments, provided are methods for preventing or reducing crop damage or post-harvest loss by a plant pathogenic microbe, said method comprising contacting a plant, a plant seed, pre- or post-harvest grain, pre- or post-harvest fruit, or pre- or post-harvest vegetable with an effective amount of composition as disclosed herein, and under conditions suitable for preventing or reducing crop damage or post-harvest loss.

In still further embodiments, provided are medical devices having antimicrobial properties, said medical device comprising a medical device in operable combination with a composition as disclosed herein, wherein said medical device comprises at least one surface that is topically coated or impregnated with the composition.

In other embodiments, provided are methods for treating, preventing, or inhibiting a microbial infection in a subject in need thereof, said method comprising administering to said subject a therapeutically effective amount of a composition as disclosed herein under conditions and for a duration suitable for treating, preventing, or inhibiting said microbial infection.

In yet other embodiments, provided are plant parts that are at least partly coated with the composition as disclosed herein.

These and other related aspects of the present disclosure will be better understood in light of the following drawings and detailed description, which exemplify certain aspects of the various embodiments.

### DETAILED DESCRIPTION

In certain embodiments, the present disclosure provides compositions enriched for cationic and antimicrobial Defensin peptide folding variants 1 (DEF_PFV1) having a defined set of disulfide bonding pairs, adopting a distinct conformation and/or having unique tertiary structures, possessing improved antimicrobial activities, and, optionally, exhibiting a desired resistance or sensitivity to proteolytic cleavage in comparison to other DEF_PFV2 with different disulfide bonding pairs. In certain embodiments, the present disclosure provides cationic and antimicrobial Defensin peptide folding variants 1 (DEF_PFV1) having a defined set of disulfide bonding pairs, exhibiting a desired resistance or sensitivity to proteolytic cleavage in comparison to other DEF_PFV2 with different disulfide bonding pairs. As disclosed herein, cationic and antimicrobial DEF_PFV1 can be purified from heterogeneous mixtures containing multiple (i.e. two or more) Defensin peptide folding variants and the desired purified DEF_PFV1 can be used advantageously in the compositions, methods, and devices as disclosed herein.

These and other aspects of the present disclosure can be better understood by reference to the following non-limiting definitions.

### Definitions

As used herein, the terms “microbe,” “microbes,” and “microbial” are used to refer to bacteria, fungi (including yeast), and oomycetes.

As used herein, the phrases “susceptible microbe (or microbes),” “susceptible microbial infection,” and the like refer to microbes that infect plants, humans, and non-human animals, or microbial infections thereof, that are subject to inhibition of microbial growth by antimicrobial peptides, including Defensin peptides, variants, and multimers thereof.

As used herein, the phrases “combating microbial damage,” “combating or controlling microbial damage,” or “controlling microbial damage” refer to reduction in damage to a plant, such as an ornamental plant or a crop plant, or to an ornamental plant or crop plant product due to infection by a microbial pathogen. More generally, these phrases refer to reduction in the adverse effects caused by the presence of a pathogenic microbe in the crop plant. Adverse effects of microbial growth are understood to include any type of plant tissue damage or necrosis, any type of plant yield reduction, any reduction in the value of the crop plant product, and/or production of undesirable microbial metabolites or microbial growth by-products including to mycotoxins.

As used herein, the phrases “inhibiting growth of a plant pathogenic microbe,” “inhibit microbial growth,” and the like refer to methods that result in any measurable decrease in microbial growth, where microbial growth includes but is not limited to any measurable decrease in the numbers and/or extent of microbial cells, spores, conidia, or mycelia. As used herein, “inhibiting growth of a plant pathogenic microbe” is also understood to include any measurable decrease in the adverse effects cause by microbial growth in a plant. Adverse effects of microbial growth in a plant include any type of plant tissue damage or necrosis, any type of plant yield reduction, any reduction in the value of the crop plant product, and/or production of undesirable microbial metabolites or microbial growth by-products including but not limited to mycotoxins. As used herein, the phrase “inhibition of microbial growth” and the like, unless otherwise specified, can include inhibition in a plant, human or animal.

As used herein, the term “antimicrobial peptide” as used herein refer to peptides, in particular Defensin peptides, which exhibit any one or more of the following characteristics of inhibiting the growth of microbial cells, killing microbial cells, disrupting or retarding stages of the microbial life cycle such as spore germination, sporulation, or mating, and/or disrupting microbial cell infection, penetration or spread within a plant or other susceptible subject, including a human, livestock, poultry, fish, or a companion animal (e.g., dog or cat).

As used herein, the term “Defensin peptide” refers to a peptide comprising a conserved gamma core peptide. Plant Defensins have been previously characterized as comprising a conserved GXCX3-9C gamma core peptide sequence (SEQ ID NO: 455), where X is any amino acid residue (Lacerda et al.) or a conserved GXCX3-10C variant gamma core peptide sequence (SEQ ID NO: 456), where X is any amino acid residue. Therefore, as used in this disclosure, a plant Defensin or C-terminal peptide comprising fragment thereof can comprise a conserved GXCX3-9C or GXCX3-10C gamma core peptide sequence, where X is any amino acid residue. Defensin peptides include proteins that are antimicrobial, that can permeabilize plasma membranes, that can bind phospholipids, that can bind sphingolipids, or that exhibit any combination of those properties. A Defensin peptide can be naturally occurring or non-naturally occurring (e.g., synthetic and/or chimeric).

As used herein, the term “Defensin peptide variant” refers to a modified Defensin peptide comprising either: (i) a conserved gamma core peptide and at least one amino acid substitution in a source Defensin peptide; and/or (ii) a modified gamma core variant sequence GXC₅X3-9 (F/W/Y) (SEQ ID NO: 457), or GXC₅X3-10 (F/W/Y) (SEQ ID NO: 458), wherein the C₆ residue is substituted with F, W, or Y. In certain embodiments, Defensin peptide variants provided herein are less than full length Defensin peptides (e.g., peptides comprising, consisting essentially of, or consisting of: (i) 30 amino acid residues or less; or (ii) 15, 16, or 17 to 30 amino acid residues).

The phrase “reference Defensin C-terminal peptide” is used herein to refer to a less than full length Defensin peptide comprising a conserved GXCX3-9C gamma core peptide sequence (SEQ ID NO: 455) or a conserved GXCX3-10C variant gamma core peptide sequence (SEQ ID NO: 456) and the two additional conserved cysteine residues located C-terminal to the gamma core peptide sequence, wherein the cysteine located closest to the N-terminus of the reference Defensin C-terminal peptide corresponds to the cysteine located closest to the N-terminus of the gamma core sequence of SEQ ID NO: 455 or SEQ ID NO: 456.

As used herein, the phrase “cation-tolerant” refers to a Defensin peptide or a variant thereof that exhibits equivalent in vitro antifungal or antimicrobial activity or no more than about a 1.5-, 2-, 3-, or 4-fold decrease in in vitro antifungal or antimicrobial activity in the presence of 100 mM KCl or 100 mM NaCl as compared to the antifungal activity of the Defensin peptide or the variant thereof in the absence of KCl or NaCl.

As used herein, the phrase “consensus sequence” refers to an amino acid sequence created by aligning two or more homologous sequences and deriving a new sequence having the conserved set of alternative amino acid residues of the homologous sequences at each position in the peptide sequence.

As used herein, the term “peptide variant” refers to any peptide with antimicrobial activity comprising one or more non-conservative amino acid substitutions in a Defensin peptide. A Defensin peptide variant can, in addition to such non-conservative substitutions, further comprise a peptide having conservative amino acid substitutions, deletions of one to five amino acids from the N-terminus, and internal deletions of one or more amino acid residues in a Defensin peptide variant provided herein.

The phrases “percent identity” or “sequence identity” as used herein refer to the number of elements (i.e., amino acids or nucleotides) in a sequence that are identical within a defined length of two protein segments in an alignment resulting in the maximal number of identical elements, and is calculated by dividing the number of identical elements by the total number of elements in the defined length of the aligned segments and multiplying by 100.

As used herein, the term “heterologous peptide” or “heterologous protein” refers to peptide, such a Defensin peptide that is not found in a cell or organism as the cell or organism occurs in nature. As such, heterologous peptides include peptides that are localized in a subcellular location, extracellular location, or expressed in a tissue that is distinct from the subcellular location, extracellular location, or tissue where the peptide or protein is found in a cell or organism as it occurs in nature.

As used herein, the term “amino acid” refers to an organic compound that contains amino (—NH₃) and carboxylate (—CO₂) functional groups, along with a side chain (R group) specific to each amino acid. In polypeptides are in certain instance referred to herein by one letter amino acid codes as follows: G—Glycine (Gly); P—Proline (Pro); A—Alanine (Ala); V—Valine (Val); L—Leucine (Leu); I—Isoleucine (Ile); M—Methionine (Met); C—Cysteine (Cys); F—Phenylalanine (Phe); Y—Tyrosine (Tyr); W—Tryptophan (Trp); H—Histidine (His); K—Lysine (Lys); R—Arginine (Arg); Q—Glutamine (Gln); N—Asparagine (Asn); E—Glutamic Acid (Glu); D—Aspartic Acid (Asp); S—Serine (Ser); or T—Threonine (Thr).

As used herein, the terms “acidic” or “anionic” are used interchangeably to refer to amino acids such as aspartic acid and glutamic acid.

As used herein, the terms “basic” and “cationic” are used interchangeably to refer to amino acids such as arginine, histidine, and lysine.

The phrases “percent identity” or “sequence identity” as used herein refer to the number of amino acids in a sequence that are identical within a defined length of two peptide or protein segments in an alignment resulting in the maximal number of identical elements and is calculated by dividing the number of identical elements by the total number of elements in the defined length of the aligned segments and multiplying by 100.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

The practice of the present disclosure will employ, unless indicated specifically to the contrary, conventional methodology and techniques that are in common use in the fields of microbiology, molecular biology, structural biology, and enzymology, which methodology and techniques are well known by and readily available to those having skill of the art. Such methodology and techniques are explained fully in laboratory manuals as well as the scientific and patent literature. See, e.g., Sambrook, et al., “Molecular Cloning: A Laboratory Manual” (2nd Edition, Cold Spring Harbor Laboratory Press, Cold Spring Harbor, N.Y., 1989); Maniatis et al., “Molecular Cloning: A Laboratory Manual” (1982); “DNA Cloning: A Practical Approach, vol. I & II” (Glover, ed.); “Oligonucleotide Synthesis” (Gait, ed., 1984); Ausubel et al. (eds.), “Current Protocols in Molecular Biology” (John Wiley & Sons, 1994); “Nucleic Acid Hybridization” (Hames & Higgins, eds., 1985); “Transcription and Translation” (Hames & Higgins, eds., 1984); “Animal Cell Culture” (Freshney, ed., 1986); and Perbal, “A Practical Guide to Molecular Cloning” (1984). All publications, patents and patent applications cited herein, whether supra or infra, are hereby incorporated by reference in their entirety.

### Antimicrobial Defensin Peptide Folding Variant (DEF_PFV) Compositions

Within certain embodiments, the present disclosure provides compositions comprising a cationic antimicrobial Defensin (DEF) peptide folding variant (PFV). The compositions according to these embodiments (1) include a DEF peptide folding variant 1 (DEF_PFV1) having a desired antimicrobial activity and/or resistance/sensitivity to proteolysis and (2) exclude a DEF peptide folding variant 2 (DEF_PFV2) having an undesired reduction in antimicrobial activity and/or an undesired resistance/sensitivity to proteolysis.

Representative cationic antimicrobial Defensin peptides that can be used advantageously in the compositions disclosed herein are presented in Table 1. Variants of such defensin peptides comprising amino acid insertions, deletions, or substitutions can also be used in the compositions provided herein.

TABLE 1
DEFENSIN PEPTIDES
8-CYSTEINE

| | | | | | | | |
|---|---|---|---|---|---|---|---|
| 8-CYS | C1-10AA-C2 | IS1 | C3-3AA-C4 | IS2 | C5-5-6AA- | IS3 | C7-3AA- |
| DEFENSIN PEPTIDES | Motif | (5 AA) | Motif | (9-11 AA) | C6 Motif | (1 AA) | C8 Motif |

DEFENSIN SEQUENCES 1

| | | | | | | | |
|---|---|---|---|---|---|---|---|
| >CAN.G1084.24 | CTTPPRRYIGVC | WSDTS | CRKVC | VEMENFEDGY | CSKIQRKC | L | CTWTC |
| >CAN.G1084.27 | CKTKSKYFEGLC | WVDSS | CRKVC | IEKDKFEDGH | CSKLLRNC | L | CTKIC |
| >CAN.G534.6 | CKTTSKHFKGLC | FADSK | CRKVC | IQEDKFEDGH | CSKLQRKC | L | CTKNC |
| >CAN.G584.62 | CKTESQKFKGLC | ITKSS | CRKAC | LKEKFTDGH | CSKLQRRC | L | CTKPC |
| >CAN.G584.63 | CKTESQKFKGLC | ITKSS | CRKAC | LKEKFTDGH | CSKLQRRC | L | CTKPC |
| >CAN.G584.64 | CKTKSKKFEGLC | FLDSS | CRKVC | VEQENFEDGH | CSKLRRLC | L | CTKLC |
| >CAN.G584.65 | CKTKSKYFTGLC | WTDSS | CRKVC | IEKDKFQDGH | CSKLQRNC | L | CTKIC |
| >CAN.G584.67 | CKTKSKYFEGLC | WVDSS | CRKVC | IEKDKFEDGH | CSKLLRNC | L | CTKIC |
| >NITAb4.5_0001626G0010 | CKTESNTFPGLC | ITKPP | CRKAC | ISEGFTDGH | CSKILRRC | L | CTKPC |
| >NITAb4.5_0001626G0020 | CKTESNTFPGLC | ITKPP | CRKAC | ISEGFTDGH | CSKILRRC | L | CTKPC |
| >NITAb4.5_0001882G0010 | CKAVSNTLSGLC | ITKPP | CREAC | ISEGFTDGK | CSKILKRC | I | CHKPC |
| >NITAb4.5_0003765G0030 | CKAESNTFPGIC | ITKPP | CRKAC | LSEGFTDGK | CSKILRRC | I | CYKPC |
| >NITAb4.5_0005347G0010 | CKTESNTFPGIC | ITKPP | CRKAC | ISEKFTDGH | CSKLLRRC | L | CTKPC |
| >PEAxI162SCF00005G00041 | CKAECATWEGIC | INKAP | CVKCC | KEQPENFTDGH | CSKILRRC | L | CTKPC |
| >PEAxI162SCF00297G00815 | CKAECPTWDGIC | INKGP | CVKCC | KAQPEKFTDGH | CSKVLRRC | L | CTKPC |
| >PEAxI162SCF00297G00816 | CKAECPTWDSVC | INKKP | CVACC | KKAKFSDGH | CSKILRRC | L | CTKEC |
| >SoLYC07G006380.3 | CKAPSQTFPGLC | FMDSS | CRKYC | IKEKFTGGH | CSKLQRKC | L | CTKPC |
| >SoLYC07G016095.1 | CKSTSQTFKGLC | FTDSS | CRKSC | LKEEFEGGH | CSKLQRKC | L | CTKIC |
| >SoLYC11G028040.2 | CKSTSQTFKGLC | FTDSS | CRKAC | VTEEFTGGH | CSKLQRKC | L | CTKVC |
| >SoLYC11G028070.2 | CKSTSQTFKGLC | FTDSS | CRKAC | VTEEFTGGH | CSKLQRKC | L | CTKVC |
| >SoPEN07G002470 | CKAPSQTFPGLC | FMDSS | CRKYC | IKEKFTGGH | CSTLQRRC | L | CTKPC |
| >SoPEN11G012530 | CKSTSQTFKGLC | FIDSS | CRKAC | VTEEFTGGH | CSKLQRKC | L | CTKVC |

DEFENSIN SEQUENCES 2

| | | | | | | | |
|---|---|---|---|---|---|---|---|
| >CC02_G09620 | CSKPSTYFFGPC | VRRST | CRRAC | SHENYPDGK | CSRFLGKC | I | CYKPC |
| >EL10AC1G02051 | CRKPSMYFSGAC | FSDTN | CQKAC | NREDWPNGK | CLVGFKC | E | CQRPC |
| >EL10AC1G02052 | CKKPSKFFKGAC | GRDAD | CEKAC | DQENWPGGV | CVPFLRC | E | CQRSC |
| >EL10AC2G04690 | CAKPSKYYKGGC | FATISVSA | CKKTC | SRENWPDGA | CVYPFRC | E | CRRPC |
| >EL10AC2G04691 | CAKPSKYYKGGC | ESTISVSA | CKKTC | SRENWPDGD | CVFPYRC | E | CRRPC |
| >EL10AC4G07858 | CAKPSKFYRGPC | FNSISVKP | CTQRC | SQENWPIGV | CTKDLRC | E | CQRPC |

DEFENSIN SEQUENCES 3

| | | | | | | | |
|---|---|---|---|---|---|---|---|
| A01P29870 | CETSSQLFNGPC | LSTTN | CANIC | QNEGFPDGD | CKGFRLRC | I | CNRPC |
| A02P32650 | CESQSHREKGPC | VSENN | CANVC | HNEGFGGGK | CRGLRRRC | F | CTRHC |
| A02P42920 | CQSRSHHERSIC | LSNHN | CANIC | RGERFSGGR | CHGLHRKC | Y | CTRLC |
| A06P43790 | CESPSTKFQGVC | LNAKN | CAGVC | TGEGFTGGQ | CSSLKC | Y | CTKTC |
| A06P43800 | CESKSHRFKGTC | VSSTN | CGNVC | HNEGFGGGK | CRGFRRRC | Y | CTRHC |
| A09P23810 | CESKSHKFRGPC | VSRHN | CANVC | HNEGFHGGK | CRGFRRRC | Y | CTRHC |
| AA31LG2G7820 | CVSKSEKFKGVC | LINTN | CAHVC | QTEGFPDGD | CRGSVRRRC | F | CIRPC |
| AA31LG2G7830 | CESKSHRFKGPC | VSTHN | CGNVC | HNEGFGGGK | CRGFRRRC | Y | CTRHC |
| AA31LG4G12330 | CASKSEKFKGSC | VSNTN | CANIC | HTEGFPEGA | CRGLRHHC | E | CTRPC |
| AA31LG5G3920 | CESKSHEFKGFC | TKWRI | CRDVC | ITEGFTDGG | CHGVLRHC | V | CKKPC |
| ACTINIDIA36588 | CESKSHGFKGAC | LIDHN | CALVC | RNEGFSGGK | CRGFRHRC | E | CTKIC |
| ACTINIDIA39325 | CTAPSSTFKYLC | ISDQN | CSSAC | EQEGFTSGE | CEGLRRRC | I | CGKPC |
| AL2G15360 | CETSSNLENGPC | LSSSN | CANVC | HNEGFSDGD | CRGFRRRC | L | CTRPC |
| AL4G27280 | CETKSLNYRGLC | MKWRN | CKRVC | ISEGFPDGR | CKGFFNNKC | V | CRKPC |
| AL5G11370 | CASOSQRFKGKC | VSDTN | CANVC | HNEGFPGGD | CRGFRRRC | F | CTRNC |
| AL5G11380 | CESKSHRFKGPC | VSTHN | CANVC | HNEGFGGGK | CRGFRRRC | Y | CTRHC |
| AL5G11390 | CESPSNKFQGVC | LNSQS | CAKAC | PSEGFSGGR | CSSLRC | Y | CSKAC |
| AL5G11400 | CKTRSDRFSGVC | LSTNN | CAIIC | QQFEHFDGGH | CEFDGALRRC | M | CTKQC |
| AL8G14820 | CETQSINFRGMC | TKWRN | CQRVC | MSEGFPDGR | CKGFIRKC | I | CTKPC |
| AL8G40800 | CQSKSHHFKYMC | TSSHN | CAIVC | RNEGFSGGR | CHGFHRRC | Y | CTRLC |
| AQoxY3G04170 | CESQSHKFKGTC | IRKSN | CASVC | QSEGENGGH | CRGVTRRC | Y | CTAKC |
| .GNM1.ANN1.4H5G | CESKSHRFKGMC | VHERN | CASVC | NLEGESGGK | CRGFRRRC | F | CTRHC |
| .GNM1.ANN1.IDU4 | CASQSHREKGVC | LSDTN | CASVC | KTEGFPSGD | CHGFRRRC | F | CTKHC |
| .GNM1.ANN1.UPL4 | CESKSHRFKGMC | VHERN | CASVC | NLEGFSGGK | CRGFRRRC | F | CTRHC |
| .GNM1.ANN1.X2H0 | CQSKSHMFKGGC | WGDHN | CALVC | RNEGFSGGR | CRGFRRRC | F | CTKLC |
| AT1G61070 | CETSSNLFNGPC | LSSSN | CANVC | HNEGFSDGD | CRGFRRRC | L | CTRPC |
| AT2G02100 | CESQSHREKGTC | VSASN | CANVC | HNEGFVGGN | CRGFRRRC | F | CTRHC |
| AT2G02120 | CASQSQRFKGKC | VSDTN | CENVC | HNEGFPGGD | CRGFRRRC | F | CTRNC |
| AT2G02130 | CESKSHRFKGPC | VSTHN | CANVC | HNEGFGGGK | CRGFRRRC | Y | CTRHC |
| AT2G02140 | CESPSNKFQGVC | LNSQS | CAKAC | PSEGFSGGR | CSSLRC | Y | CSKAC |
| AT2G02147 | CKTRSDRESGVC | ISTNN | CAIIC | QQFEHFDGGH | CEFDGAFRRC | M | CTKQC |
| AT2G31953 | CETKSLNYRGLC | LKWRS | CKRVC | ISEGFPDGR | CKGFFNNKC | V | CRKPC |
| AT2G31957 | CEAKSLDWKGMC | LKWRN | CRQVC | ISEGFTDGR | CKGFTRKC | I | CSKPC |
| AT5G63660 | CQSKSHHFKYMC | TSNHN | CAIVC | RNEGFSGGR | CHGFHRRC | Y | CTRLC |
| ATRu.CHR11.1065 | CESQSHKFKGTC | VSKSN | CAHVC | QTEGFPGGH | CRGFRRRC | E | CAKNC |
| ATRu.CHR11.1068 | CESQSHKFKGTC | LSKSN | CANVC | KTEGFPGGH | CRGFRRRC | F | CTKKC |
| BCAB04G20312 | CETSSQLENGPC | LSTTN | CANIC | QNEGFPDGD | CHGFRLRC | I | CNRPC |
| BCAB07G30354 | CESQSHREKGTC | VSENN | CANVC | HNEGFGGGK | CRGFRRRC | F | CTRNC |
| BCAB07G30710 | CESKSHKFRGPC | VSRHN | CANVC | HNEGFHGGK | CRGFRRRC | Y | CTRHC |
| BCAC03G17116 | CESQSHREKGPC | VSDNN | CANVC | HNEGFGGGK | CRGLRRRC | F | CTTHC |
| BCAC04G21530 | CESKSHKERGLC | VSRHN | CANVC | HNEGFHGGK | CRGFRRRC | Y | CTRHC |
| BCAC06G31249 | CESKSHRFKGTC | VSTTN | CANVC | HNEGFPGGK | CRGFRRRC | F | CTRHC |
| BCAC06G34079 | CESKSHREKGTC | VSSTN | CGNVC | HNEGFGGGK | CRGFRRRC | Y | CTRHC |
| BCAC09G49338 | CETSSQLENGPC | LSTTN | CANIC | QNEGFPDGD | CKGFRLRC | I | CNRPC |
| BoLC1T03737H | CETSSQLENGPC | LSTTN | CANIC | QNEGFPDGD | CKGFRLRC | I | CNRPC |
| BoLC2T11102H | CESQSHREKGPC | VSDNN | CANVC | HNEGFGGGK | CRGLRRRC | F | CTRHC |
| BoLC2T12358H | COSRSHHERSIC | LSNHN | CANIC | RGERFSGGR | CHGLHRKC | Y | CTRLC |
| BoLC7T43808H | CESKSHRFKGTC | VSSTN | CGNVC | HNEGFGGGK | CRGFRRRC | Y | CTRHC |
| BoLC9T55933H | CESKSHKFRGLC | VSRHN | CANVC | HNEGFHGGK | CRGFRRRC | Y | CTRHC |
| BRAA01T03115Z | CETSSQLENGPC | LSTTN | CANIC | QNEGFPDGD | CKGFRLRC | I | CNRPC |
| BRAA02T08280Z | CESQSHREKGPC | VSENN | CANVC | HNEGFGGGK | CRGLRRRC | F | CTRHC |
| BRAA02T09214Z | CQSRSHHERSIC | LSNHN | CANIC | RGERFSGGR | CHGLHRKC | Y | CTRLC |
| BRAA06T27257Z | CESKSHREKGTC | VSSTN | CGNVC | HNEGFGGGK | CRGFRRRC | Y | CTRHC |
| BRAA09T37986Z | CESKSHKFRGPC | VSRHN | CANVC | HNEGFHGGK | CRGFRRRC | Y | CTRHC |
| C01P37750 | CETSSQLENGPC | LSTTN | CANIC | QNEGFPDGD | CKGFRLRC | I | CNRPC |
| C02P50240 | CESQSHREKGPC | VSDNN | CANVC | HNEGFGGGK | CRGLRRRC | F | CTRHC |
| C02P62860 | CQSRSHHFRSIC | LSNHN | CANIC | RGERFSGGR | CHGLHRKC | Y | CTRLC |
| C07P32970 | CESKSHRFKGTC | VSSTN | CGNVC | HNEGFGGGK | CRGFRRRC | Y | CTRHC |
| C07P32980 | CESPSTKFQGVC | LNAKN | CAGVC | TGEGFTGGQ | CSSLKC | Y | CTKTC |
| C09P30680 | CESKSHKFRGLC | VSRHN | CANVC | HNEGFHGGK | CRGFRRRC | Y | CTRHC |
| CAN.G1126.33 | CESASQRENGYC | VRSSN | CASIC | STEGFTDGK | CKGTIRRHC | T | CIRNC |
| CAN.G186.35 | CISQSHGFKGPC | GHDHN | CALVC | RNEGFSGGD | CIGVFIRKC | Y | CTKNC |
| CAN.G282.87 | CEALSGNFKGLC | LSSRD | CGNVC | RREGFTDGS | CIGFRLQC | F | CTKPC |
| CAN.G458.12 | CESQSHREKGPC | VRKSN | CASVC | QTEGFTGGH | CRGFRRRC | F | CTKHC |
| CAN.G976.1 | CESKSQRFKGPC | VRQKN | CAAVC | ETEGFPGGD | CRGFRRRC | F | CTRPC |
| CAN.G976.2 | CESQSHREKGVC | ASETN | CASVC | QTEGFSGGD | CRGFRRRC | F | CTRPC |
| CAN.G976.3 | CDTLSENFKGIC | FSSTN | CATTC | QNEDEEYTGGH | CRGLHRRC | F | CTTEC |
| CAN.G976.4 | CERLSGTFKGIC | FSSKN | CASVC | ENEDEEFTGGH | CSGLRRRC | Y | CTADC |
| CANSAT54G0755 | CESKSHHFKGLC | VIDHN | CATIC | RTEGFSGGN | CQGFRQRC | F | CTRHC |
| CANSAT78G0503 | CEAQSHHFKGPC | VRKSN | CANVC | RTEGFPSGH | CRGFRRRC | F | CTKHC |
| CANSAT78G1068 | CEARSQSHRGIC | VTNHT | CDNVC | KIERFSSGY | CSGIDRRC | L | CTKHC |
| CARHR125680 | CETKSTNYRGLC | LRWRN | CKRVC | MSEGFPDGR | CRGIFDNKC | V | CRKPC |
| CARHR144740 | CESQSHREKGTC | VSASN | CGNVC | HNEGENGGN | CRGFRRRC | F | CTRHC |
| CARHR144750 | CESQSHREKGTC | TNEQN | CRNIC | HNEGFFGGK | CRGFRRRC | F | CTRNC |
| CARHR144760 | CESKSQQFRGLC | MSSTN | CATVC | HNEGFPGGE | CRGFRRRC | F | CTRPC |
| CARHR144770 | CESQSNKFKGKC | VSETN | CGNVC | RTEGFTGGD | CRGFRRRC | F | CTRNC |
| CARHR144790 | CETRSQKFKGPC | VSENN | CANVC | QTEGFPDGD | CNGILRRC | Y | CNSPC |
| CARHR144800 | CETPSTKFQGVC | LNSQN | CAKVC | PTEGASGGR | CNSLRC | V | CSKTC |
| CARHR277540 | CQSKSHHYKYMC | TSNHN | CAMVC | RNEGFSGGQ | CHGFHRRC | Y | CTRLC |
| CARub.0002S0412 | CESSSNLFSGPC | LSESN | CANVC | NTEGFPDGD | CRGFRRRC | M | CTRPC |
| CARub.0004S1326 | CETKSLYFRGMC | TKWAN | CKRVC | ISEGFPDGR | CKGFIRDC | V | CRKPC |
| CARub.0005S0117 | CESKSHRFKGPC | VSGNN | CKNVC | HNEGFPGGR | CRGFRRRC | Y | CTRHC |
| CARub.0005S0119 | CESPSSKFSGVC | LNSQT | CAKAC | PSEGFTGGR | CNSLRC | Y | CSKPC |
| CARub.0005S0120 | CKTRSDRFSGVC | LSNNN | CAIIC | QQFENFDSGH | CEFDGAFRRC | L | CTKNC |
| CARub.0008S2442 | CQSKSHHFRRMC | WSDHN | CAMIC | RSERFSGGH | CHGFHRRC | Y | CTRLC |
| CA_17033_V3 | CASQSHAFKGPC | VGDHN | CALVC | RNEGFSGGN | CQGFRRRC | F | CTKLC |
| CC06_G07880 | CESLSHREKGTC | VRGSN | CAAVC | QTEGFPGGQ | CSGLRRRC | F | CSKPC |
| CC06_G07890 | CETPSKRFKGPC | VKESN | CAAVC | QTEGFQGGK | CRGLRRRC | F | CTRP |
| CC11_G07160 | CESQSHREKGPC | IRKDN | CAAVC | QTEGFHGGR | CRGFRRRC | F | CTKHC |
| CDE04G2371 | CESPSRHYKGLC | FRSSN | CASIC | HIEHFSHGH | CHGFRRRC | I | CSRPC |
| CDE07G0627 | CESPSRHYKGLC | FRSSN | CASIC | HIEHFSHGY | CHGFRRRC | I | CSRPC |
| CFA002248 | CESQSHKFHGPC | LGDHN | CALVC | RNEGFSGGD | CKGLRRRC | F | CTRLC |
| CICLEV10006362M.G | CESQSHREKGPC | VSKSN | CAAVC | QTEGFHGGH | CRGFRRRC | F | CTKRC |
| CICLEV10026887M.G | COSQSHHFHGAC | FSHHN | CAFVC | RNEGFSGGK | CRGARRRC | F | CSKLC |
| CIPAW.04G178300 | CESKSHGFQGMC | TRDHN | CALVC | RNEGFSGGK | CRGFRHRC | F | CTKLC |
| CIPAW.04G178400 | CESKSHKFQGPC | VRDNN | CGLVC | KNEGFTGGK | CRGFRRRC | F | CTKIC |
| CLCG02G000130 | CKSQSHSFHGPC | MRDHN | CAVVC | RTEGFSGGD | CVGFRRRC | F | CTRLC |
| COL.COLO4_16172 | CESKSHWFKGPC | WGDHN | CALVC | RNEGFSGGR | CRGFRHRC | F | CTKLC |
| COL.COLO4_16210 | COSQSHGFKGGC | LSNHN | CALVC | RNEGFSGGQ | CRGLRHRC | F | CTRRC |
| CPA.G.SC2.96 | CESQSHREKGAC | GSRSN | CAAVC | QTEGFHGGH | CRGFRRRC | F | CTKHC |
| CSS0006682 | CESKSHGFKGPC | MTDHN | CALVC | RNEGFSGGK | CRGVRHRC | F | CTKLC |
| CSS0011856 | CESKSHGFKGPC | MTDHN | CALVC | RNEGFSGGK | CRGVRHRC | F | CTKLC |
| DCAR_019911 | CEVRSAYFVSLC | LIDHH | CEMVC | KVEGFPTGK | CLGLVPHC | I | CLGTC |
| DCAR_023524 | CESQSHKFKGTC | LSETN | CGNVC | KNEGFSGGN | CRGLRRRC | F | CTRHC |
| DCAR_026845 | CESQSHKFKGTC | VSRSN | CANVC | KNEGFPGGH | CRGFRRRC | F | CIKHC |
| DINV20716 | CEAQSHGFKGAC | MSNHN | CALVC | RNEGFSGGK | CRGFRHRC | F | CTKQC |
| DuzIb213G0741 | CESKSHRFKGPC | LSHHN | CGMVC | KNEGFSGGK | CRGVRRRC | F | CTRRC |
| DuzIb248G0383 | CESQSHREKGAC | VSENN | CAAVC | QTEGELGGN | CRGFRRRC | F | CTKQC |
| DuzIb248G1477 | CESQSHRFKGMC | VRESN | CATVC | QTEGFQGGH | CRGFHRRC | F | CTKHC |
| DuzIb251G0835 | CESQSHREKGTC | LRKSN | CAAVC | QTEGFHGGH | CRGFRRRC | F | CTKHC |
| DuzIb268G0537 | CESKSHKYKGAC | FHDHN | CAYVC | RNEGFSGGK | CHGLHHHC | F | CTKIC |
| DuzIb268G0639 | CESKSHKFKGPC | MGDHN | CAYVC | RNEGFSGGK | CRGLRHRC | F | CTKIC |
| ECA233G3610 | COSQSHMYHGAC | LRDHN | CALVC | RNEGFSGGR | CRGFRRRC | F | CTRLC |
| EL10AC5G10881 | CTNPSKTYKGPC | VSDTN | CESVC | QSEGFPQGN | CHGFRRRC | I | CGKPC |
| EL10AC5G10882 | CETASHKFKGPC | LGDRN | CANVC | QTEGFPGGD | CQGLRRRC | F | CTRPC |
| EL10AC5G10884 | CDAPSHSFKGIC | TRKRN | CESVC | NSEGFADGD | CKGFRRRC | I | CNRPC |
| EL10AC5G10885 | CVTPSHRERGIC | VSKRN | CESAC | NSEGFPDGN | CKGFRRRC | I | CNRPC |
| EL10AC7G17637 | CKKVPSRTYKGPC | ARDAN | CRSVC | ISEGYNGGD | CHGFRRRC | | CTDKC |
| EuCGR.B02620 | CESQSQRFKGAC | VSKTN | CASVC | QTEGFHGGH | CRGFRRRC | F | CTKHC |
| EuCGR.F03380 | CESQSHKFHGLC | MSDRT | CVVVC | KEEDFDDGK | CRGFRHRC | F | CRKPC |
| EuCGR.H05052 | CESQSHGFHGSC | VSGHN | CALVC | RNEGFSGGR | CRGFRRRC | F | CTKLC |
| FAN04G0442 | CESQSHGFHGTC | IRAHN | CALVC | RHEGFSGGR | CRGFRRRC | F | CTRRC |
| FAN07G0709 | CETLSHKFKGTC | LRESN | CASVC | QTEGFTGGD | CRGLRRRC | F | CTKHC |
| FAN08G1943 | CESQSHGFYGTC | IHAHN | CALVC | RHEGFSGGR | CRGFRRRC | F | CTRRC |
| FAN11G1290 | CKVPSKTFKGMC | FSQKN | CIVRC | HSEGYGWGE | CSHLRRRC | M | CYKPC |
| FAN16G3227 | CEVPSKTFTGLC | FRDTH | CIVRC | HSEGYGYGK | CSHVLRRC | R | CLKAC |
| FAN16G3645 | CKVPSKTFKGMC | FSQKN | CIVRC | HSEGYGWGE | CSHLRRRC | M | CYKPC |
| FAN18G0803 | CESQSHGFHGTC | IRAHN | CALVC | RHEGFSGGR | CRGFRRRC | F | CTRRC |
| FAN19G2445 | CESPSAKFEGPC | IMGSN | CALTC | QKEGHDGGF | CRDFWLPKC | F | CTKPC |
| FAN22G0227 | CESLSHKFQGTC | LRESN | CASVC | QTEGFTGGD | CRGLRRRC | F | CTKHC |
| FAN23G2500 | CEPPSAKFEEPC | IMGSN | CALTC | QKEGHDGGF | CHDFWLPKC | F | CTKPC |
| FAN25G0202 | CEVPSKTFTGLC | FRDTH | CIVRC | HSEGYGYGK | CSHVLRRC | R | CLKAC |
| FAN26G3046 | CESPSAKFEGPC | IMGSN | CALTC | QKEGHNGGF | CRDFWLPKC | F | CTKPC |
| FAN28G2126 | CESPSGKFEGPC | IMGSN | CALTC | QKEGHDGGF | CRDFWLPKC | F | CTKPC |
| FVH4_1G21320 | CESPSAKFEGPC | IMGSN | CALTC | QKEGHDGGF | CRDFWLPKC | F | CTKPC |
| FVH4_2G25190 | CETQSIKYEGMC | SREKP | CASLC | QTKGYSRGE | CHGVYKIC | V | CATQC |
| FVH4_3G14600 | CKVPSKTFKGMC | FSQKN | CIVRC | HSEGYGWGE | CSHLRRRC | > | CYKPC |
| FVH4_3G14620 | CEIPSKKFKGLC | IRDRK | CIVIC | HSEGYGYGK | CSHIGRRC | M | CLKTC |
| FVH4_3G40491 | CESLSHNENGLC | LSESN | CASVC | ATEGFTGGD | CRGLRRRC | F | CTKQC |
| FVH4_4G36102 | CETLSHKFKGTC | LRESN | CASVC | QTEGFTGGD | CRGLRRRC | F | CTKHC |
| FVH4_4G36104 | CESLSHKFKGTC | LSETN | CASVC | KTEGFSGGD | CRGLRRRC | F | CTKHC |
| FVH4_5G33001 | CESQSHGFHGTC | IRAHN | CALVC | RHEGFSGGR | CRGFRRRC | F | CTRRC |
| GLYMA.05G198000 | CLTRRHGFQGRC | LEDRQ | CAHVC | RSDGFIGGQ | CRGPLRKC | F | CSRPC |
| GLYMA.06G160300 | CESQSHGFHGLC | NRDHN | CALVC | RNEGFSGGR | CKGFRRRC | F | CTRIC |
| GLYMA.06G160400 | CESKSHRFKGMC | LSKHN | CASVC | HLEGFTGGK | CRGFRRRC | F | CTRHC |
| GLYMA.08G005300 | CSSKSHGFKGLC | FSDTN | CAHVC | RTEGFTGGD | CGGFRRRC | F | CTRIC |
| GLYMA.16G100400 | CESQSHREKGPC | LSDTN | CGSVC | RTERFTGGH | CRGFRRRC | F | CTKHC |
| GoHIR.A01G033900 | CKATSNVYKGLC | LIDDS | CDIVC | KREGFHKGN | CKGFLRKC | V | CTNPC |
| GoHIR.A01G044800 | CETKSGKFKGMC | MSSTN | CASVC | KSEPGFDGGH | CRGFRRQC | V | CTKPC |
| GoHIR.A01G044900 | CETQSGKFKGMC | MSSTN | CASVC | KSEPGFDGGY | CRGFRRQC | F | CTKPC |
| GoHIR.A01G045100 | CETKSSEYKGIC | LFDAN | CDSIC | KVEPSFDGGH | CHGFFRRC | Y | CTKPC |
| GoHIR.A01G045300 | CQSRSTEYRGMC | LEDVN | CDNIC | KTEPGFTGGH | CHSFFRSC | Y | CTKPC |
| GoHIR.A03G115800 | CESPSHKFKGMC | MNRDN | CAIVC | QTEGYEDGK | CEGFRRRC | I | CIKPC |
| GoHIR.A03G115938 | CDSPSKKFNGMC | MNHDN | CATVC | QTESHEDGK | CEGFYRRC | I | CIKPC |
| GoHIR.A05G375500 | CESKSHRFKGVC | LSDHN | CGLVC | RNEGELDGW | CRGFRHRC | F | CTRNC |
| GoHIR.A09G211400 | COSQSHKFHGAC | VSSHN | CAMVC | RDEGFSGGR | CRGFRRRC | F | CTKLC |
| GoHIR.D01G023520 | CESPSNAFKGLC | LRDDN | CDIVC | KTEGFPNGD | CKGFLRKC | V | CTKPC |
| GoHIR.D01G023526 | CESPSNAFKGLC | LRDDN | CDIVC | KTEGFPNGD | CKGFLRKC | V | CTKPC |
| GoHIR.D01G035700 | CETQSGKFKGMC | MSSTN | CASVC | KSE PGFDGGH | CQGFRRQC | V | CTKPC |
| GoHIR.D01G035800 | CVAESGKFKGMC | MSSTN | CASVC | KSE PGFDGGH | CQGFRRRC | L | CTKPC |
| GoHIR.D01G035900 | CETKSSEYKGIC | LEDAN | CDSIC | KVEPGFDGGH | CHGFFRRC | Y | CTKPC |
| GoHIR.D01G036000 | CVAESGKFKGMC | MSSTN | CASVC | KSEPDFDGGH | CQGFRRRC | L | CTKPC |
| GoHIR.D01G036100 | CETKSSEYKGIC | LFDAN | CDSIC | KIESGEDGGH | CHGFFRRC | Y | CTKPC |
| GoHIR.D01G036200 | CESKSGKFKGMC | FLDAN | CDSIC | KAEPGSTGGH | CHGIFRIC | Y | CTKPC |
| GoHIR.D02G139400 | CESPSHKFKGMC | MNRDN | CATVC | QTEGYEDGK | CEGFRRRC | I | CIKPC |
| GoHIR.DO4G040300 | CESKSHRFKGVC | LSDHN | CGLVC | RNEGFLDGW | CRGFRHRC | F | CTRNC |
| GoHIR.D09G204900 | COSQSHKFHGAC | LSSHN | CAMVC | RNEGFSGGR | CRGFRRRC | F | CTKLC |
| GoRAI.002G029500 | CESRSNAFKGLC | LRDNN | CDIVC | KTEGFPNGG | CKGFIRKC | V | CTKPC |
| GoRAI.002G029600 | CKATSNIYKGLC | LLDDS | CDIVC | KREGFHSGN | CKGFLRKC | V | CTNPC |
| GORAI.002G029700 | CESPSNAFKGLC | LRDDN | CDIVC | KTEGFPNGD | CKGFLRKC | V | CTKPC |
| GoRAI.002G043900 | CEVESGKFKGMC | MSSTN | CASVC | KSEPGFDGGH | COGERRRC | L | CTKPC |
| GoRAI.002G044100 | CEVESGKFKGMC | MSSTN | CASVC | KSEPGFDGGH | CQGFRRRC | L | CTKPC |
| GoRAI.002G044200 | CETKSSEYKGIC | LFDAN | CDSIC | KIEPGEDGGH | CHGFFRRC | Y | CTKPC |
| GoRAI.005G157500 | CESPSHKFKGMC | MNRDN | CATVC | QTEGYEDGK | CEGFRRRC | I | CIKPC |
| GoRAI.006G228900 | CQSQSHKFHGAC | LSSHN | CAMVC | RNEGFSGGR | CRGFRRRC | F | CTKLC |
| GSVIVG01000230001 | CESQSHKFEGAC | MGDHN | CALVC | RNEGFSGGK | CKGLRRRC | F | CTKLC |
| GSVIVG01010274001 | CESQSHREKGTC | VRQSN | CAAVC | QTEGFHGGN | CRGFRRRC | F | CTKHC |
| HANXRQCHR12G0365811 | CESPSHKFKGIC | LSDTN | CSSVC | QSEGFPWGK | CIGFRRRC | F | CLREC |
| HANXRQCHR12G0365821 | CESQSHKFKGTC | LSDTN | CANVC | HSERFSGGK | CRGFRRRC | F | CTTHC |
| HANXRQCHR15G0469151 | COSQSHMFHGTC | MRDHN | CALVC | RNEGFSGGK | CRGFRRRC | F | CTRLC |
| HANXRQCHR17G0533321 | CESQSQEFHGSC | MSNHN | CGLVC | RTEGESAGI | CRGVRGRC | F | CLKTC |
| HMA1.2P1_0383F.1_G140490 | CESKSNRFKGPC | ARDSN | CATVC | QTEGFYGGN | CKGFRRRC | F | CTKNC |
| HMA1.2P1_0383F.1_G140500 | CESKSHRFKGPC | VSATN | CGSVC | QTEGFHGGN | CRGFRRRC | F | CTKHC |
| HuND02690 | CRDLSHKFKGFC | VRDRN | CANVC | STEGFTNGH | CDGFRRRC | F | CSRPC |
| HuND02692 | CETPSHKFQGIC | ISSRN | CASTC | ASEHFPGGY | CRGLRRRC | M | CTRPC |
| HuND09734 | CETPSRKERGIC | VSRTN | CANVC | KTEGFPGGR | CRGFRRRC | F | CLEHC |
| HUND09739 | CESPSYSFKGIC | VSRTN | CANVC | KTEGFPGGR | CRGLRRRC | F | CLKHC |
| HUND09741 | CESRSYRFKGIC | VSRTN | CANVC | KTEGFPGGR | CRGVRRRC | F | CLKHC |
| HUND18397 | CETPSHKFKGIC | VSRTN | CANVC | KTEGFPAGR | CRGFRRRC | F | CLTHC |
| HUND22171 | CESKSHHYKGMC | LRDHN | CAMVC | RTEGFSGGH | CIGARRRC | F | CTRGC |
| LALb_CHR05G0229081 | CESQSHREKGAC | LSDTN | CGSVC | HNEGENGGH | CRGFRRRC | F | CTKHC |
| LALb_CHR07G0183991 | CGKVLSSRFKGKC | LTDHN | CDSVC | QLEGYVGGD | CHGVRRRC | F | CNKKC |
| LALb_CHR08G0237751 | CESKSHRFKGLC | HSDHN | CASIC | LVEGFTSGK | CHGFRQRC | F | CTKVC |
| LALb_CHR11G0068851 | CESQSHLFKGPC | SRDHN | CALVC | RNEAFSGGR | CKGFRRRC | F | CTKLC |
| LALb_CHR12G0202751 | CDPVLSSRFKGKC | FSDHN | CNSVC | QLEGYTDGN | CHGLRQRC | F | CNRKC |
| LALb_CHR12G0202761 | CQSKSHREKGPC | LSYSN | CGHVC | RNEGFSGGR | CRGLRRRC | F | CTKNC |
| LALb_CHR12G0206521 | CKSKSQRYKGSC | YSDNN | CAIIC | EGEGFTGGK | CHGFFCKC | W | CKRPC |
| LALb_CHR16G0391851 | CSTKSDRERGTC | FRSSN | CAAVC | NSENFPSGT | CRGFPGTC | Y | CTSNC |
| LALb_CHR16G0392511 | CESRSHKLKGFC | YSSKR | CAHVC | TSHHNFIGGH | CRGFIRRC | H | CTKPC |
| LALb_CHR22G0357361 | CKYESNKFHGVC | LSDTN | CESVC | QTEGFPFGK | CDGVLPRTC | V | CKKPC |
| Lj1G0008875 | CESKSHRFHGLC | LSHHN | CASVC | HLEGFTGGK | CRGFRKRC | F | CKKRC |
| Lj1G0011574 | CQSQSHRERGGC | WGDHN | CALVC | RNEGFSGGR | CRGFRRRC | F | CTRLC |
| Lj4G0007075 | CETKSHT FHHSC | VSGHN | CAQVC | KTEGFTGGK | CRGFRRRC | F | CTKRC |
| Lj4G0025743 | CESQSHREKGPC | VSDTN | CASVC | HGEKFSGGH | CRGFRRRC | F | CTKHC |
| LSAT_1_V5_GN_3_2_7700 | CRYSSKTYKGLC | FYSRN | CDIIC | KAEGAPGGGH | CQILTLRC | F | CNVC |
| MANES.02G163900 | CESQSHREKGVC | VRKSN | CAAIC | QTEGFHGGH | CRGLRRRC | F | CTKHC |
| MANES.05G122400 | CESKSHHERGLC | TIAHN | CAMVC | RNEGFSGGK | CRGFRRRC | F | CTRLC |
| MANES.07G074400 | CESKSHHFRGVC | TIAHN | CAIVC | RNEGFSGGK | CKGFRRRC | F | CTRLC |
| MANES.18G077612 | CESQSHKFKGIC | LSDTN | CASVC | KTEGFTGGD | CKGARRRC | F | CTRQC |
| MANES.18G077618 | CESQSHREKGMC | TRHSN | CAAIC | QTEGFHAGH | CRGLRRRC | F | CTKHC |
| MD00G1135500 | COSQSHGFHGTC | LRHHN | CALVC | RNEGFSGGR | CRGLRRRC | F | CTRLC |
| MD02G1207600 | CEFPSTLFKGLC | FSSNN | CKHTC | RKEQFTRGK | CSLLTRTC | M | CTKKC |
| MD02G1301000 | CESQSNRFKGTC | VSKSN | CAAVC | QTEGFPGGN | CRGLRRRC | F | CTKHC |
| MD02G1301100 | CESHSRKYKGTC | LGKSN | CASVC | QTEGFHGGH | CRGFRRRC | F | CTKHC |
| MD05G1234500 | CEVPSKTFRGLC | FSDTH | CIVKC | HSEGFGYGK | CSHVLRQC | R | CLKAC |
| MD11G1108400 | CEVPSTLFKGLC | ISSNN | CKHTC | RKEQFTRGH | CSVFTRAC | V | CTKKC |
| MD11G1108800 | CEVPCTLFKGLC | FSSNN | CKHTC | RKEQFTRGH | CSVFTRAC | V | CTKKC |
| MD11G1113600 | CEYPSTLFKGLC | FSSNN | CKHTC | RKEQFTRGH | CSVFTRAC | V | CTKKC |
| MD12G1061400 | CESPSHKYRGIC | FRSSN | CATTC | KKEGFMGGK | CAGFRMRC | V | CTKKC |
| MD12G1061700 | CESPSHKYRGIC | FRSSN | CATTC | KKEGFMGGK | CAGFRMRC | V | CTKKC |
| MD12G1121400 | CEVPSTLFKGLC | ISSNN | CKHTC | RKEQFTRGK | CIGDGFTRKC | V | CTKKC |
| MD13G1011400 | CEAASGKFKGLC | FSSTN | CKNTC | KREQFTGGK | CRGFRRRC | M | CNKKC |
| MD13G1011500 | CEAASGKFKGLC | FSSTN | CKNTC | KVEKFTGGQ | CQGFRRRC | M | CNKKC |
| MD13G1011600 | CEATSGKFKGMC | FSSTN | CKNTC | KTEKFIGGE | CHGFRRRC | I | CNKKC |
| MD13G1011700 | CEVPSTLFKGLC | FSSNN | CKHTC | RKEQFTRGK | CSGLTRTC | V | CTKKC |
| MD13G1011800 | CEVPSTLFKGLC | FSSNN | CKHTC | RKEQFTRGK | CSGLTRTC | M | CTKKC |
| MD13G1012000 | CEVPSTLFKGLC | FSSNN | CKHTC | RKEQFTRGK | CSGLTRTC | M | CTKKC |
| MD13G1012100 | CEAASGKFKGLC | FSSNN | CKHTC | KREQFTGGK | CRGFRRRC | M | CNKKC |
| MD13G1012400 | CDSQSHRFKGSC | VSKSN | CATVC | QTEGFRGGH | CRGFRRRC | E | CTKHC |
| MD14G1061400 | CESPSHKYHGIC | FRSSN | CATTC | KKEGFMGGK | CTGFWMRC | V | CTKKC |
| MD14G1061900 | CESPSHKFHGIC | FRSSN | CATTC | KKEGFMGGK | CTGFRMRC | V | CTKKC |
| MD14G1062000 | CESRSHKYHGPC | FRFIFSSN | CAVTC | KGEGFTGGK | CKGFHMRC | I | CTKQC |
| MD14G1062100 | CESPSQKYPGIC | RSSY | CAAIC | KQQGLMGGK | CTGFRRKC | V | CTKKC |
| MD14G1073000 | CEFPSTLFKGLC | FSSNN | CKHTC | RKEQFTRGK | CSLLTRTC | M | CTKKC |
| MD16G1008800 | CDSHPSSKFKGVC | FFTNN | CVDTC | KLEGSSGGQ | CRGFRRRC | I | CTKQC |
| MD16G1008900 | CEAASGKFKGMC | ESSNN | CANTC | AREKFDGGK | CKGFRRRC | M | CTKKC |
| MD16G1009000 | CESQSNRFKGSC | VSKSN | CAAVC | QTEGFPGGN | CRGLRRRC | F | CTKHC |
| MD16G1009100 | CESQSRKFKGTC | LSKTN | CASVC | QTEGFPGGN | CRGFRRRC | E | CTKHC |
| MEDTR3G032570 | CDSQSHSFKGVC | WIKHN | CANVC | KTEGFTGGH | CHGFRRRC | E | CSKPC |
| MEDTR3G097390 | CREKSRLFEELC | FNSED | CANTC | RYEGFHLGGK | CWGLFRTC | Y | CKKKC |
| MEDTR3G097420 | CESKSHREKGMC | MSDHN | CASVC | HVEGFPGGN | CRGFRRRC | F | CKKRC |
| MEDTR8G070770 | CESQSHKFKGPC | ASDHN | CASVC | QTERFSGGH | CRGFRRRC | F | CTTHC |
| MEDTR8G070780 | CETPSNSFKGAC | FSDTN | CASVC | QTEGFPGGH | CKGFRQRC | | CTKPC |
| MEDTR8G070790 | CETPSNNEKGLC | VSDTN | CASVC | QTEGFPGGH | CEGFRQRC | F | CTKPC |
| MEDTR8G075980 | CLSQSHSFKGLC | LSDQN | CATVC | LTEGFTDGR | CRGFRQRC | F | CSKPC |
| MEDTR8G095370 | CESKSHKFKGPC | SRDSN | CASVC | RGEGFTGGD | CRGFRRRC | F | CTRNC |
| MEDTR8G095375 | CESQSHKFKGPC | VSDSN | CGSVC | RGEGFIGGD | CRGVRHRC | F | CTRNC |
| MELO3C005215.2 | CESPSHNFKGLC | FSDTN | CGNIC | KTEGFSGGL | CRGFRRRC | F | CTKHC |
| MELO3C023203.2 | CQSQSHHFHGTC | IRDHN | CALVC | RTEGESGGE | CVGFRRRC | F | CTHRC |
| MIGuT.E00476 | CKYKSKKFWGVC | LSDIR | CTDVC | KREGFQGGD | CQGIRRRC | L | CYNRC |
| MIGuT.E00478 | CEAKSHREKGIC | ARKAN | CAAVC | QTEGFHGGH | CRGFRRRC | F | CTKHC |
| MIGuT.E00740 | CESKSASYKGVC | VLDVT | CKLAC | LNEGFADGD | CEGVRRRC | M | CRRPC |
| MIGuT.H01662 | CETKSSIFTGAC | FSDRN | CETIC | EKEGELNGR | CKLWKC | I | CSKDC |
| MIGuT.H01663 | CETQSHGFKGQC | WSEHN | CRLVC | AHEGFRGGK | CRGFRKRC | F | CTRPC |
| MIGuT.J01199 | CLSQSQRFRGPC | VSDTN | CRLVC | HNEGFSGGD | CQGFRRRC | F | CTKHC |
| MIGuT.L00621 | CNYESKKFWGIC | LSDSK | CGEVC | TKEGFEGGD | CKGLRRRC | Y | CFNNC |
| MIGuT.M01514 | CETPSRYENGIC | FSRRN | CGAIC | EKEGFSSGR | CKAFKC | V | CGKDC |
| MIGuT.M01515 | CESQSHGFKGRC | LSDHN | CGMVC | KNEGFSNGW | CRGFRQRC | F | CTRAC |
| MIGuT.M01516 | CTSKSSNFKGLC | FSSHN | CKAVC | NGENFPDGK | CHGFKRRC | F | CQKPC |
| MIGuT.M01517 | CESQSFNFRGPC | VIDHN | CKAVC | ESEGFTDGD | CQGFRRRC | F | CRKPC |
| MSTRG.13083 | CEKPSRLYNGSC | TEERL | CIEIC | QKEAYPSGR | CDLLKC | M | CINKC |
| NITAb4.5_0000374G0050 | CESQSHREKGPC | SRDSN | CATVC | LTEGFSGGD | CRGFRRRC | F | CTRPC |
| NITAb4.5_0000374G0060 | CESQSQRFRGLC | VREKN | CAAVC | ETEGFSGGD | CRGLRRRC | F | CTRPC |
| NITAb4.5_0004431G0020 | CMSPSHGYHGPC | WHDHN | CALVC | RNEGFSGGD | CVGIQRKC | Y | CTKLC |
| NITAb4.5_0007878G0020 | CESQSHRFHGTC | VRGSN | CASVC | QTEGFIGGN | CRGLRRRC | F | CTRNC |
| NITAb4.5_0009181G0010 | CESKSQRFRGPC | VREKN | CAAVC | ETEGFSGGD | CRGLRRRC | F | CTRPC |
| NN3G18726 | CESQSHREKGIC | VRKSN | CAAVC | QTEGFHGGH | CRGFRRRC | F | CTKHC |
| NN7G38190 | CKTPSRQFRGLC | LIGRN | CDNVC | RGEGFPDGD | CDGFRRRC | I | CSRPC |
| NN7G38191 | CDTPSQKFVGLC | LIDRN | CENVC | KTEGFPAGH | CEGLRRRC | F | CSEPC |
| NN7G38263 | CDTPSRLFRGLC | LINRN | CDNTC | RVEGFPDGH | CKGLRRRC | F | CSRPC |
| OEu059528.1 | CESLSHREKGPC | VRNAN | CAAVC | QTEGFPGGH | CRGFRRRC | F | CTKPC |
| OEu059529.1 | CESLSHRFKGPC | VRRGN | CAAVC | QTEGFPGGL | CRGFRRRC | F | CTKHC |
| OS01G0932700 | CKARSKMYRGKC | RGNRN | CAMIC | VHEEYTGGY | CSKGVFSKC | M | CTKRC |
| OS02G0172300 | CHDHSQTFKGMC | FHTSN | CIACC | TNEGYTGGY | CKPFTYRC | M | CTKDC |
| OS02G0212100 | CEKPSSHFKGLC | LRSQN | CDNEC | MLEGYMDGK | CKYLTRRC | I | CSMEC |
| OS02G0629800 | CLSQSHRFKGMC | VSSNN | CANVC | RTESFPDGE | CKSHGLERKC | F | CKKVC |
| OS03G0130300 | CESQSHREKGPC | ARKAN | CASVC | NTEGFPDGY | CHGVRRRC | M | CTKPC |
| OS04G0522100 | CVSQSHRFVGAC | MRKSN | CEHVC | MTEGFPWGE | CRFHGIERKC | F | CKKRC |
| PEAxI162SCF00025G00313 | CESQSHREKGPC | VSKSN | CASVC | QTEGFHGGH | CRGLRRRC | F | CTKHC |
| PEAxI162SCF00072G01012 | CLSPSHRYKGMC | LRDHN | CALVC | RNEGFSGGD | CIGFRRRC | F | CTKLC |
| PEAxI162SCF00088G00119 | CESQSHRFHGTC | VRESN | CASVC | QTEGFIGGN | CRGFRRRC | F | CTRNC |
| PEAxI162SCF00088G01110 | CETPSQRFKGPC | VSDTN | CANVC | QTEGFPGGD | CQGLRRRC | F | CTKPC |
| PEAxI162SCF00088G01111 | CETLSHRFRGPC | VREKN | CAAVC | ETEGFSGGD | CRGLRRRC | F | CTRPC |
| PEAxI162SCF00120G01114 | CMSPSHGYRGPC | IRDHN | CALVC | RDEGFSGGD | CVGIQRKC | Y | CTKLC |
| PEAxI162SCF00376G00824 | CEHRSQHYYGAC | FANNS | CDSMC | KCDGFDGGF | CRLTNC | F | CHVKC |
| | | NMNNN | | | | | |
| PEAxI162SCF00376G00828 | CQYKSKTFHGVC | VTGNT | CNQKC | QSEAFDGGR | CQGVRHRC | Y | CYRTC |
| PEAxI162SCF00376G00829 | CRTNSKRYSGPC | ENNNN | CDFIC | RRCEGFEGGH | CSSRTC | V | CTKSC |
| | | NRNQN | | | | | |
| PGR044G1171 | CESQSHKFRGTC | VSNTN | CGSVC | QTEGFHGGH | CRGFRRRC | F | CTKQC |
| PGR102G0532 | CESKSHRFRGVC | MSNSN | CASVC | HNEGFPGGR | CRGVRKRC | F | CTKVC |
| PGSC0003DMG400015129 | CESQSHREKGPC | VSEKN | CASVC | ETEGFSGGD | CRGLRRRC | F | CTRPC |
| PGSC0003DMG400015130 | CESLSHREKGPC | TRDSN | CASVC | ETERFSGGN | CHGFRRRC | F | CTKPC |
| PGSC0003DMG400015132 | CESQSRRFKGLC | FSKSN | CGSVC | HTEGFSGGH | CRGFRRRC | F | CTRHC |
| PGSC0003DMG400029484 | CESQSHKYKGPC | VRKSN | CGAVC | QTEGFTGGH | CRGVRRRC | E | CTKHC |
| PGSC0003DMG400030676 | CESQSHSFKGPC | VGDTN | CASVC | QTEGFIGGD | CRGLRRQC | F | CTRNC |
| PGSC0003DMG400030677 | CESQSNRFKGTC | VRDSN | CATVC | QTEGFIGGN | CRGFRRRC | F | CTRNC |
| PGSC0003DMG400032549 | CISQSHRYKGPC | LRDHN | CALVC | RNEYFSGGD | CIGFGFHRKC | Y | CTKFC |
| PHVuL.002G278400 | CFTKKEGWGRC | IADFT | CARSC | RNVGYIGGK | CQGLTRRC | E | CVLNC |
| PHVuL.002G278500 | CRTKSRKYTGAC | YSNDI | CANYC | KGDGFDDGD | CGGLSHIC | E | CTKNC |
| PHVuL.002G278600 | CESPSHSFKGLC | FNDRN | CANVC | LTEGFTGGK | CEGARHRC | F | CTIIC |
| PHVuL.002G278700 | CWSESQAFKGLC | FSNDN | CATVC | LTEGFTGGK | CEGLRRRC | F | CSKNC |
| PHVuL.003G282400 | CESRSRHLRGKC | ESDEN | CASAC | RTERFSGGH | CRGFRCRC | F | CDKRS |
| PHVuL.003G282500 | CESQSHREKGPC | VSDTN | CASVC | RTERFSGGH | CRGFRRRC | F | CTKHC |
| PHVuL.009G158000 | CESQSHGFKGAC | TGDHN | CALVC | RNEGFSGGN | CRGFRRRC | F | CTKIC |
| PHVuL.009G158101 | CESKSHRFKGMC | LSDRN | CASVC | HLEGFTGGN | CRGFRRRC | F | CTRHC |
| PoTRI.004G138100 | CLSQSHHYKGPC | LRGHN | CAMVC | KTEGFAGGE | CKGFISRC | F | CAKLC |
| PoTRI.T011200 | CLSQSHSFKGPC | VRGHN | CASVC | KTEGFPGGE | CKGFRRRC | F | CAKPC |
| PoTRI.T011201 | CLSQSHSFKGPC | VRGHN | CASVC | KTEGFPGGE | CKGFRRRC | F | CAKPC |
| PoTRI.T011300 | COSQSHYFKGPC | ARDHN | CAWVC | RNEGFSGGR | CKGFRRRC | F | CTKLC |
| PoTRI.T011301 | CQSQSHYFKGPC | ARDHN | CAWVC | RNEGFSGGR | CKGFRRRC | F | CTKLC |
| PRuPE.1G201200 | COSQSHGFRGPC | IRHHN | CALVC | RNEGFSGGR | CRGFRRRC | F | CTRLC |
| PRuPE.1G341500 | CESQSNRFKGTC | VSTSN | CASVC | QTEGFPGGH | CRGFRRRC | E | CTKHC |
| PRuPE.1G341600 | CDSQSQKFKGMC | FLTSS | CVQSC | KSEGFTGGQ | CRGFRRRC | F | CSKNC |
| PRuPE.1G341700 | CESQSQKFKGIC | FLSSN | CATSC | TTEGENGGQ | CRGFRRRC | F | CSKAC |
| PRuPE.4G131400 | CEVTSKTFIGLC | IKDRN | CMVRC | HSEGYGYGK | CSHILRKC | R | CLKPC |
| PRuPE.7G070800 | CESLSTKFKGFC | LRSSR | CAEAC | QKEGFMGGK | CPGFRLRC | T | CTKKC |
| PRuPE.7G070900 | CESLSTKFKGFC | LRSSR | CAEAC | QKEGFMGGK | CPGFRLRC | T | CTKKC |
| PRuPE.7G071000 | CESLSTKFKGPC | IRSSN | CANIC | EEEGFKGGK | CVGFRLRC | T | CTKNC |
| PRuPE.7G071100 | CESLSTKFKGPC | IRSSN | CANIC | EEEGFKGGK | CVGFRLRC | T | CTKNC |
| PRuPE.7G071200 | CESLSTKFKGPC | IRSSN | CANIC | EEEGFKGGK | CVGFRLRC | T | CTKNC |
| PRuPE.7G071300 | CESLSTKFKGPC | IRSSN | CANIC | EEEGFKGGK | CVGFRLRC | T | CTKNC |
| PRuPE.7G071400 | CESLSTKFKGPC | FRSSN | CANIC | EKEGFKGGK | CVGFRLRC | T | CTKNC |
| PRuPE.7G073300 | CESLSTKFKGLC | FRSSN | CADIC | KKEGFLEGK | CAGFRLRC | T | CTKKC |
| PSAT4G112000 | CESKSHKFKGTC | LSDTN | CASVC | QTERFTGGH | CRGFRHRC | Y | CTTHC |
| PSAT4G112240 | CESPSQKFKGLC | FSDTN | CASVC | QTEGFPGGK | CEGLRHRC | F | CTKPC |
| PSAT4G112280 | CESPSQKFKGVC | LSDRN | CASVC | QTEGFTGGD | CKGLRQRC | F | CTKPC |
| PSAT4G112360 | CESPSQIFKGLC | FTDTN | CASVC | HTEGFPGGK | CKGLPKRC | I | CTKPC |
| PSAT4G112680 | CPSPSTKFKGVC | VSDFD | CMITC | QMEGLSEGK | CQSLKC | I | CAKPC |
| PSAT5G057240 | CQSKSSQFKGIC | LSNKK | CARVC | HLEGFLEGGK | CKGKLLKHC | S | CKKSIC |
| PSAT7G040440 | CKVKSTKFRGPC | SFDKN | CATVC | RGEGYRDGD | CHGLRRRC | M | CLC |
| PSAT7G041040 | CVSQSHRFHGPC | LRDSN | CATVC | LGEGFTGGD | CHGFRRRC | E | CSRFC |
| PSO541G0662 | CESPSHREKGIC | VRKSN | CAAIC | QTEGFSSGK | CRGFRSRC | V | CIKHC |
| PSO541G3846 | CESASHREKGIC | VRKSN | CGAVC | QTEGFSGGK | CRGLRARC | M | CTKHC |
| PSO675G5343 | CDSKSHKERGLC | VSDTN | CRNVC | QTERFSDGH | CRGARRRC | Y | CQNPC |
| PSO675G6539 | CESASHREKGIC | VRKSN | CGAVC | QTEGFSGGK | CRGLRARC | M | CTKHC |
| QL03P041430 | CESQSHREKGPC | VRKSN | CASVC | QTEGFHGGQ | CRGFRRRC | F | CTKHC |
| QL10P038692 | CESQSHKFKGAC | MSNHN | CALVC | RNEGFSGGR | CRGFRHRC | E | CTRIC |
| RCHM_V2.0_CHR2G0115151 | CDSPSGKFKGPC | IISSN | CALTC | QKEGFAGGH | CRDFLRKC | Y | CAKPC |
| RCHM_V2.0_CHR4G0445761 | CETLSHKFKGTC | LRESN | CASVC | QTEGFSGGD | CRGLRRRC | F | CTKHC |
| RCHM_V2.0_CHR4G0445781 | CESQSLKYEGMC | LRESH | CASVC | QTEGYSGGD | CHGLYSIC | V | CTKDCQ |
| RCHM_V2.0_CHR4G0445791 | CESQSHKFAGIC | LIESN | CASIC | KTEGYSGGN | CGGFRRRC | F | CTKDC |
| RCHM_V2.0_CHR4G0445801 | CESQSHKFEGIC | LKQSN | CASIC | KTEGYSGGN | CRGFRRRC | E | CTKDC |
| RCHM_V2.0_CHR4G0445851 | CESQSHKFAGIC | LIESN | CASIC | KTEGYSGGN | CGGFRRRC | F | CTKDC |
| RCHM_V2.0_CHR5G0024061 | CEVPSKTFSGLC | FSDTH | CIVRC | HSEGYGYGK | CSHILRRC | R | CLKAC |
| RCHM_V2.0_CHR5G0024081 | CEVPSKTFKGLC | VKDRN | CVVRC | HSEGYGNGK | CSHVRRQC | M | CLKIC |
| RCHM_V2.0_CHR5G0024091 | CEYPSRTFKGLC | VKDKN | CVVRC | RSEGYNYGK | CSHVRRRC | R | CFKPC |
| RCHM_V2.0_CHR7G0227171 | CESQSHGFHGTC | IRAHN | CALVC | RHEGFSGGR | CRGFRRRC | F | CTRRC |
| RCHM_V2.0_CHR7G0231101 | CESRSHGFHGTC | IHHNN | CALVC | RNEGFPGGR | CRGFRRRC | E | CTRNC |
| SALBOW2G2204 | CESKSHREKGIC | VRKAN | CAAVC | QTEGFHSGH | CRGFRRRC | Y | CTRHC |
| SALBoW6G1343 | CESESSSFKGLC | LRNAD | CYIVC | QTEGFSGGK | CSC | E | CTKPC |
| SALBOW6G2289 | CESESSTFKGLC | LKNAD | CYIVC | RTEGFSGGK | CSSFLRRC | E | CTKPC |
| SALBoW7G0339 | CSAASRLFSGLC | FSDSN | CSSIC | EKEGELTGV | CKGLQC | I | CQKDC |
| SALBoW8G0100 | COSQSHKFKGKC | FSDKN | CGSVC | QTEGFPSGK | CHGLRQRC | E | CTRPC |
| SALBoW8G2012 | CSAPSKLFRGVC | LLRSN | CSSIC | ATEGFLYGL | CKDLKC | V | CYKDC |
| SALBOW8G2299 | CSAASRLFSGLC | FSDNN | CSAIC | EKEGELSGV | CKGLKC | V | CQKDC |
| SALBoW8G3014 | CSAASRLFSGLC | FSDNN | CSAIC | EKEGELSGV | CKGLKC | V | CQKDC |
| SALBOW8G3771 | CSAPSRLFRGLC | FRRSN | CTSIC | EKEGFLYGL | CKGLKC | V | CYKDC |
| SALBoW8G5093 | CQSQSHKFKGKC | FSDKN | CGSVC | QTEGFPSGK | CHGLRQRC | E | CTRPC |
| SALBoW8G5419 | CSAPSKLFRGVC | LLRSN | CSSIC | ATEGFLYGL | CKDLKC | V | CYKDC |
| SC20G0000730 | COSQSHLFRGPC | VRNHN | CASVC | QNEGFSDGS | CRGFRRRC | E | CAKFC |
| SED0017845 | CESPSRHFKGVC | FSRNN | CGHVC | KTEGFHGGH | CRGFRRRC | E | CTKHC |
| SED0025601 | CESQSHHFKGLC | FSKNN | CGHVC | KTEGFHGGH | CRGFRRRC | E | CTKHC |
| SEGI 34828 | CKSQSHTFKGYC | VSDSN | CKNVC | RTEKFPTGG | CDFHVASRKC | Y | CYKPC |
| SoLYC04G008470.3 | CESQSHRYKGPC | VRKNN | CANVC | KTEGFSGGH | CRGFRRRC | F | CAKHC |
| SoLYC07G007710.4 | CESQSHHFKGNC | LSDTN | CGSVC | RTEGFTGGN | CRGFRRRC | E | CTRNC |
| SoLYC07G007730.4 | CESQSNSFKGTC | VRDSN | CATVC | QTEGFIGGN | CRGFRRRC | F | CTRNC |
| SoLYC07G007735.1 | CEPQSHSFKGPC | SRDSN | CATVC | QTEGFIGGD | CRGLRRQC | E | CTTEC |
| SoLYC07G007750.3 | CESQSHREKGPC | VSEKN | CASVC | ETEGFSGGD | CRGFRRRC | E | CTRPC |
| SoLYC11G006260.2 | CMSPSHSYHGPC | WHDHN | CAIVC | RNEGFSGGN | CVGIQLKC | Y | CTKLC |
| SoLYC11G006950.3 | CISQSHRYRGPC | VLDHN | CAIIC | RNEGFSGGD | CIGWRRRC | F | CTKLC |
| SoPEN04G003620 | CETQSHKYKGPC | VRKSN | CANVC | KTEGFRGGH | CRGFRRRC | F | CTKNC |
| SoPEN04G028660 | CQYHSETFHGVC | VTGNN | CNRHC | QRENFEGGR | CHGIRHYRC | V | CYRTC |
| SoPEN07G001020 | CESLSHREKGPC | ASDKN | CASIC | ETERFSGGN | CRGFRRRC | F | CTKPC |
| SoPEN07G001030 | CESQSHREKGPC | VSEKN | CASVC | ETEGFSGGD | CRGFRRRC | F | CTRPC |
| SoPEN07G001050 | CESQSHSFKGPC | SRDSN | CATVC | QTEGFIGGD | CRGLRRQC | E | CTTDC |
| SoPEN07G001060 | CESQSNSFKGTC | VRDSN | CATVC | QTEGFIGGN | CRGFRRRC | E | CTRNC |
| SoPEN07G003700 | CESQSHHFKGNC | LSDTN | CGSVC | RTEGFTGGN | CRGFRRRC | F | CTRNC |
| SoPEN11G002190 | CMSPSHSYHGPC | WHDHN | CAIVC | RNEGFSGGN | CVGIQLKC | Y | CTKLC |
| SoPEN11G002870 | CISQSHRYRGPC | VLDHN | CAIIC | RNEGFSGGD | CIGWRRRC | F | CTKLC |
| SP2G13520 | CESKSHRFRGVC | VSDGN | CGNVC | HNEGFGGGK | CRGFRRRC | F | CTRHC |
| SP2G13530 | CESQSHREKGKC | LSDGN | CGNVC | HNEGFGGGK | CRGFRRRC | F | CTRNC |
| SP2G13540 | CESPSGKYHGVC | LNAKN | CASVC | PSEGFTGGH | CKSLRC | Y | CIKTC |
| SP2G13550 | CETLSKVFKGMC | WNONN | CAIMC | REFEHFHSGH | CRGFLRRC | Y | CTKRC |
| THA.LOC104818404 | CQSKSHHFHRTC | VSNHN | CAMVC | RTEGFSGGH | CVGFRRRC | F | CTRLC |
| THA.LOC109116172 | CESPSHREKGAC | LSETN | CANVC | HNEGFIGGN | CRGLRRRC | F | CTKHC |
| THECC.02G298100 | CQSQSHREKGMC | MRKSN | CAAVC | QTEGFHSGH | CRGFHRRC | F | CTKHC |
| THECC.04G140800 | CESKSHAFKGPC | LSDHN | CGLVC | RNEGFPDGW | CRGIRRRC | E | CTRRC |
| THECC.04G141100 | CESKSHGFKGAC | LSDHN | CGMVC | SNEGFSGGR | CRGFRHRC | F | CTRRC |
| THECC.04G177500 | CESKSHKFKGPC | LGDHN | CALVC | RNEGFSGGR | CRGFRHRC | F | CTKLC |
| THHALV10005228M.G | CESKSHRFKGAC | LSETN | CKNVC | HNEGFRGGN | CRGLRRRC | F | CTRNC |
| THHALV10005229M.G | CESKSHRFKGKC | LSETN | CKNVC | HNEGFTGGN | CRGFRRRC | E | CTRNC |
| THHALV10005233M.G | CESASGKFQGVC | LNSKS | CASVC | QSNEGFSGGH | CNSLRC | Y | CTKTC |
| THHALV10005235M.G | CQSKSHHFKHMC | WSDHN | CAIIC | RNEGFSGGR | CRGFHRRC | Y | CTRLC |
| THHALV10017555M.G | CKARSINFTALC | LRWSK | CKQAC | LTENFPDGR | CIARRC | E | CMTPC |
| THHALV10017942M.G | CEARSINFRGMC | MKWRN | CQRVC | MSEGFPDGR | CAGFIRRC | I | CRKPC |
| THHALV10023787M.G | CESSSQLENGPC | LSTTN | CANIC | HNEGFTDGD | CQGFRRRC | I | CTRPC |
| TPR.G19837 | CESQSHKFKGPC | ASDHN | CASVC | QTERFPGGH | CRGFRRRC | F | CTTHC |
| TPR.G29340 | CESKSHRFKGMC | LSDHN | CASVC | HVEGFPGGN | CRGFRRRC | F | CKKRC |
| TPR.G7787 | CESKRRWIYGNC | LSDEK | CASDC | LVKGFLFGK | CRGYDNRC | Y | CKKPRC |
| TWI75G0548 | CASKSHYYKGPC | LHNHN | CAMVC | RNEGFSGGK | CHGLGRRC | F | CTKLC |
| TWI79G1670 | CLSPSHKFHGTC | VRGHN | CAEIC | RTEGFSGGD | CHGFRRRC | M | CSRRC |
| UNITIG 22.G5598 | CKYKSEKFWGIC | ITDSK | CIEVC | RREGFQTGD | CKGLRRRC | Y | CVNNC |
| ZM00001Eb002390 | CQSQSHRFRGPC | LRRSN | CANVC | RTEGFPGGR | CRGFRRRC | F | CTTHCH |
| ZM00001Eb236940 | CLSQSHAFKGVC | LSNTN | CDNVC | KTEKFTGGE | CKMDGVMRKC | Y | CKKVC |
| ZM00001Eb247810 | CLSQSHREKGLC | MSSNN | CANVC | QTENFPGGE | CKAEGATRKC | F | CKKIC |
| ZM00001Eb304240 | CESQSFRFKGPC | SRDAN | CANVC | LTEGFTGGV | CKGLRHRC | E | CTRDC |

Certain exemplary antimicrobial Defensin peptides and C-terminal fragments thereof that can be used advantageously in the compositions disclosed herein are presented in Table 2. Variants of such defensin peptides comprising amino acid insertions, deletions, or substitutions can also be used in the compositions provided herein.

TABLE 2
EXEMPLARY ANTIMICROBIAL DEFENSIN PEPTIDES

| | US Patent or | | |
|---|---|---|---|
| | US Patent | | |
| | Publication | | |
| | No. | | |
| | (each | | |
| | incorporated | | |
| | by reference | | SEQ |
| | in their | | ID |
| Name | entireties) | Sequence of mature WT defensin | NO |
| MtDef4 | U.S. Pat. | RTCESQSHKFKGPCASDHNCASVCQTERFSGGHCRGFRRRCFCTTHC | 286 |
| | No. | | |
| | 7,825,297 | | |
| MtDef4 | | GHCRGFRRRCFCTTHC | 459 |
| C-term. | | | |
| MtDef5.1 | U.S. Pat. | KLCQKRSTTWSGPCLNTGNCKRQCINVEHATFGACHRQGFGFACFCYKKC | 460 |
| a | No. | | |
| | 10,253,328 | | |
| MtDef5.1 | | GACHRQGFGFACFCYKKC | 461 |
| a | | | |
| C-term. | | | |
| MtDef5.1 | U.S. Pat. | KLCERRSKTWSGPCLISGNCKRQCINVEHATSGACHRQGIGFACFCKKKC | 462 |
| b | No. | | |
| | 10,253,328 | | |
| MtDef5.1 | | GACHRQGIGFACFCKKKC | 463 |
| b | | | |
| C-term. | | | |
| OeDefla | US | KPCTKLSKGWRGLCAPHKCSSYCIHHEGAYHGACLKNRHSKHYGCYCYYRH | 464 |
| | 20220089661 | CY | |
| OeDefla | | GACLKNRHSKHYGCYCYYRHCY | 465 |
| C-term. | | | |
| OeDef1b | US | KPCTKLSKGWHGLCAPHKCSNYCIHHEGAYHGACLKNHHNKHYGCYCYYRH | 466 |
| | 20220089661 | CY | |
| OeDef1b | | GACLKNHHNKHYGCYCYYRHCY | 467 |
| C-term. | | | |
| MtDef6 | US | RVCESQSHKFKGPCARNHNCALVCQTERFSGGRCRGFRRRCFCTRPC | 468 |
| | 20220089661 | | |
| MtDef6 | | GGRCRGFRRRCFCTRPC | 469 |
| C-term. | | | |
| OeDef7 | US | RICESLSHRFKGPCVRRGNCAAVCQTEGFPGGLCRGFRRRCFCTKHC | 318 |
| | 20220089661 | | |
| OeDef7 | | GGLCRGFRRRCFCTKHC | 470 |
| C-term. | | | |
| SbDef1 | US | VHVCTMRNKFFHGPCMSNKNCAASCIQHRIGGGGYCSSRRQICKCTLQC | 471 |
| | 20220081475 | | |
| SbDef1 | | GGGGYCSSRRQICKCTLQC | 472 |
| C-term. | | | |

Within certain aspects of these compositions, the Defensin peptide is a cationic Defensin peptide having (1) a first cysteine pair motif including C₁ and C₂, (2) a first intervening sequence (IS1), (3) a second cysteine pair motif including C₃ and C₄, (4) a second intervening sequence (IS2), (5) a third cysteine pair motif including C₅ and C₆, (6) a third intervening sequence (IS3), and (7) a fourth cysteine pair motif including C₇ and C₈. Typically, cationic Defensin peptides according to these embodiments have an isoelectric point of between 8.0 and 12.0 and/or a DEF_PFV1 exhibits greater protease resistance or greater protease sensitivity than the corresponding DEF_PFV2. Typically, these compositions are substantially free of DEF_PFV2.

According to these aspects, the cysteine residues C₁, C₂, C₃, C₄, C₅, C₆, C₇, and C₈ of a DEF_PFV1 form a first set of disulfide bonds and DEF_PFV1 thereby adopts a first conformation that exhibits a desired antimicrobial activity and preferred resistance/sensitivity to proteolysis as compared to a Defensin peptide folding variant 2 (DEF_PFV2) that has the same amino acid sequence as DEF_PFV1 but forms a distinct second set of disulfide bonds between cysteine residues C₁, C₂, C₃, C₄, C₅, C₆, C₇, and C₈ and adopts a distinct second conformation having an undesired reduction in antimicrobial activity and/or an undesired resistance/sensitivity to proteolysis.

Exemplified herein are Defensin peptide folding variant 1 (DEF_PFV1) wherein the Defensin peptide is a native Defensin peptide or a synthetic variant of a native Defensin peptide bearing one or more amino acid insertions, deletions, and/or substitutions in a native Defensin peptide, including substitutions of one hydrophobic amino acid with another hydrophobic amino acid, one cationic amino acid with another cationic amino acid, or one anionic amino acid with another cationic or anionic amino acid.

In other aspects of the present embodiments, a Defensin peptide folding variant 1 (DEF_PFV1) forms a the first set of disulfide bonds that comprise a C₁-C₈ disulfide bond, a C₂-C₅ disulfide bond, a C₃-C₆ disulfide bond, and a C₄-C₇ disulfide bond, and/or wherein Defensin peptide folding variant 2 (DEF_PFV1) forms a second set of disulfide bonds other than a C₁-C₈ disulfide bond, a C₂-C₅ disulfide bond, a C₃-C₆ disulfide bond, and a C₄-C₇ disulfide bond.

In other aspects of these embodiments, a Defensin can include a first cysteine pair motif that is a C₁-10AA-C₂ motif having in contiguous sequence from its N-terminal end ten amino acids (A₁, A₂, A₃, A₄, A₅, A₆, A₇, A₈, A₉, and A₁₀) flanked by an N-terminal C₁ and a C-terminal C₂ wherein A₁ can be an anionic amino acid selected from Asp (D) and Glu (E); wherein A₄ can be a polar neutral amino acid selected from Ser(S) and Thr (T); wherein A₇ can be an aromatic amino acid selected from Phe (F), Tyr (Y), and Trp (W); and/or wherein A₉ can be an aliphatic amino acid selected from Gly (G), Ala (A), Val (V), Leu (L), Ile (I), and Pro (P).

In related aspects of these embodiments, a Defensin can include a second cysteine pair motif that is a C₃-3AA-C₄ motif having in contiguous sequence from its N-terminal end three amino acids (A₁₁, A₁₂, and A₁₃) flanked by an N-terminal C₃ and a C-terminal C₄ wherein A₁₁ is an aliphatic amino acid selected from Gly (G), Ala (A), Val (V), Leu (L), Ile (I), and Pro (P) or a cationic amino acid selected from the group consisting of His (H), Arg (R), and Lys (K) and/or wherein A₁₃ is an aliphatic amino acid selected from the group consisting of Gly (G), Ala (A), Val (V), Leu (L), Ile (I), and Pro (P) or a polar neutral amino acid selected from the group consisting of Ser(S) and Thr (T).

In other related aspects of these embodiments, a Defensin can include a third cysteine pair motif that is a C₅-5-6AA-C₆ motif having in contiguous sequence from its N-terminal end five or six amino acids (A₁₄, A₁₅, A₁₆, A₁₇, A₁₈ and, optionally, A₁₉) flanked by an N-terminal C₅ and a C-terminal C₆ wherein A₁₄ is a cationic amino acid selected from His (H), Arg (R), and Lys (K) or an aliphatic amino acid selected from Gly (G), Ala (A), Val (V), Leu (L), Ile (I), and Pro (P) or a polar neutral amino acid selected from Ser(S) and Thr (T); wherein A₁₆ is an aliphatic amino acid selected from Gly (G), Ala (A), Val (V), Leu (L), Ile (I), and Pro (P) or an aromatic amino acid selected from Phe (F), Tyr (Y), and Trp (W); wherein A₁₈ is a cationic amino acid selected from the group consisting of His (H), Arg (R), and Lys (K).

In other related aspects of these embodiments, a Defensin can include a fourth cysteine pair motif is a C₇-3AA-C₈ motif having in contiguous sequence from its N-terminal end three amino acids (A₂₀, A₂₁, and A₂₂) flanked by an N-terminal C₇ and a C-terminal C₈, wherein A₂₀ is a polar neutral amino acid selected from the group consisting of Ser(S) and Thr (T); wherein A₂₁ is a cationic amino acid selected from the group consisting of His (H), Arg (R), and Lys (K); and/or wherein A₂₂ is an aliphatic amino acid selected from the group consisting of Gly (G), Ala (A), Val (V), Leu (L), Ile (I), and Pro (P).

In further related aspects of these embodiments, a Defensin can include a first intervening sequence (IS1) having five amino acids and/or a second intervening sequence (IS2) having from nine to eleven amino acids, and/or a third intervening sequence (IS3) having one amino acid that is selected from an aliphatic amino acid selected from Gly (G), Ala (A), Val (V), Leu (L), Ile (I), and Pro (P) or an aromatic amino acid selected from Phe (F), Tyr (Y), and Trp (W).

Within other aspects of these compositions, the Defensin peptide is a synthetic variant of a native Defensin peptide having (1) a first amino acid pair motif including X₁ and C₂, (2) a first intervening sequence (IS1), (3) a second cysteine pair motif including C₃ and C₄, (4) a second intervening sequence (IS2), (5) a third cysteine pair motif including C₅ and C₆, (6) a third intervening sequence (IS3), and (7) a fourth cysteine pair motif including C₇ and Y₁. Typically, cationic Defensin peptides according to these embodiments have an isoelectric point of between 8.0 and 12.0 and/or a DEF_PFV1 exhibits greater protease resistance or greater protease sensitivity than the corresponding DEF_PFV2. Typically, these compositions are substantially free of DEF_PFV2.

In other aspects, cysteines C₂, C₃, C₄, C₅, C₆, and C₇ of DEF_PFV1 can form a first set of disulfide bonds and DEF_PFV1 can thereby adopt a first conformation that exhibits greater antimicrobial activity as compared to a Defensin peptide folding variant 2 (DEF_PFV2) that has the same amino acid sequence as DEF_PFV1 but forms a distinct second set of disulfide bonds between cysteines C₂, C₃, C₄, C₅, C₆, and C₇ and adopts a distinct second conformation.

In further aspects, amino acid X₁ and Y₁ are independently selected from the group of amino acids other than cysteine. For example, amino acids X₁ and Y₁ can both be Ser(S) or Thr (T).

Exemplified herein are Defensin peptide folding variant 1 (DEF_PFV1) that form a first set of disulfide bonds including a C₂-C₅ disulfide bond, a C₃-C₆ disulfide bond, and a C₄-C₇ disulfide bond and/or Defensin peptide folding variant 2 (DEF_PFV2) that form a second set of disulfide bonds other than a C₂-C₅ disulfide bond, a C₃-C₆ disulfide bond, and a C₄-C₇ disulfide bond.

In other aspects of these embodiments, the synthetic Defensin peptide includes a first cysteine pair motif that is a X₁-10AA-C₂ motif having in contiguous sequence from its N-terminal end ten amino acids (A₁, A₂, A₃, A₄, A₅, A₆, A₇, As, A₉, and A₁₀) flanked by an N-terminal X₁ and a C-terminal C₂ wherein A₁ can be an anionic amino acid selected from Asp (D) and Glu (E); wherein A₄ can be a polar neutral amino acid selected from Ser(S) and Thr (T); wherein A₇ can be an aromatic amino acid selected from Phe (F), Tyr (Y), and Trp (W); and/or wherein A₉ can be an aliphatic amino acid selected from Gly (G), Ala (A), Val (V), Leu (L), Ile (I), and Pro (P).

In related aspects of these embodiments, the synthetic Defensin peptide includes a second cysteine pair motif that is a C₃-3AA-C₄ motif having in contiguous sequence from its N-terminal end three amino acids (A₁₁, A₁₂, and A₁₃) flanked by an N-terminal C₃ and a C-terminal C₄ wherein A₁₁ is an aliphatic amino acid selected from Gly (G), Ala (A), Val (V), Leu (L), Ile (I), and Pro (P) or a cationic amino acid selected from the group consisting of His (H), Arg (R), and Lys (K) and/or wherein A₁₃ is an aliphatic amino acid selected from the group consisting of Gly (G), Ala (A), Val (V), Leu (L), Ile (I), and Pro (P) or a polar neutral amino acid selected from the group consisting of Ser(S) and Thr (T).

In other related aspects of these embodiments, the synthetic Defensin peptide includes a third cysteine pair motif that is a C₅-5-6AA-C₆ motif having in contiguous sequence from its N-terminal end five or six amino acids (A₁₄, A₁₅, A₁₆, A₁₇, A₁₈ and, optionally, A₁₉) flanked by an N-terminal C₅ and a C-terminal C₆ wherein A₁₄ is a cationic amino acid selected from His (H), Arg (R), and Lys (K) or an aliphatic amino acid selected from Gly (G), Ala (A), Val (V), Leu (L), Ile (I), and Pro (P) or a polar neutral amino acid selected from Ser(S) and Thr (T); wherein A₁₆ is an aliphatic amino acid selected from Gly (G), Ala (A), Val (V), Leu (L), Ile (I), and Pro (P) or an aromatic amino acid selected from Phe (F), Tyr (Y), and Trp (W); wherein A₁₈ is a cationic amino acid selected from the group consisting of His (H), Arg (R), and Lys (K).

In yet other related aspects of these embodiments, the synthetic Defensin peptide includes a fourth cysteine pair motif is a C₇-3AA-Y₁ motif having in contiguous sequence from its N-terminal end three amino acids (A₂₀, A₂₁, and A₂₂) flanked by an N-terminal C₇ and a C-terminal Y₁, wherein A₂₀ is a polar neutral amino acid selected from the group consisting of Ser(S) and Thr (T); wherein A₂₁ is a cationic amino acid selected from the group consisting of His (H), Arg (R), and Lys (K); and/or wherein A₂₂ is an aliphatic amino acid selected from the group consisting of Gly (G), Ala (A), Val (V), Leu (L), Ile (I), and Pro (P).

In further related aspects of these embodiments, the synthetic Defensin peptide includes a first intervening sequence (IS1) having five amino acids, and/or a second intervening sequence (IS2) having from nine to eleven amino acids, and/or a third intervening sequence (IS3) having one amino acid that is selected from an aliphatic amino acid selected from Gly (G), Ala (A), Val (V), Leu (L), Ile (I), and Pro (P) or an aromatic amino acid selected from Phe (F), Tyr (Y), and Trp (W).

Within other aspects of these compositions, the Defensin peptide is a synthetic variant of a native Defensin peptide having in contiguous amino acid sequence from its N-terminal end (1) a first amino acid pair motif including X₁ and Y₁, (2) a first intervening sequence (IS1), (3) a first cysteine pair motif including C₃ and C₄, (4) a second intervening sequence (IS2), (5) a second cysteine pair motif including C₅ and C₆, (6) a third intervening sequence (IS3), and (7) a second amino acid pair motif including X₂ and Y₂. Defensin peptides according to these embodiments have an isoelectric point of between 8.0 and 12.0 and/or a DEF_PFV1 exhibits greater protease resistance or greater protease sensitivity than the corresponding DEF_PFV2. Typically, these compositions are substantially free of DEF_PFV2.

In other aspects, amino acids X₁, Y₁, X₂, and Y₂ are each independently selected from amino acids other than cysteine, and cysteines C₃, C₄, C₅, and C₆ of DEF_PFV1 form a first set of disulfide bonds and DEF_PFV1 thereby adopts a first conformation that exhibits greater antimicrobial activity as compared to a Defensin peptide folding variant 2 (DEF_PFV2) that has the same amino acid sequence as DEF_PFV1 but forms a distinct second set of disulfide bonds between cysteines C₃, C₄, C₅, and C₆ and adopts a distinct second conformation. In certain compositions, amino acids X₁, Y₁, X₂, Y₂ are each Ser(S) or Thr (T).

In further aspects, the first amino acid pair motif is an X₁-10AA-Y₁ motif having in contiguous sequence from its N-terminal end ten amino acids (A₁, A₂, A₃, A₄, A₅, A₆, A₇, As, A₉, and A₁₀) flanked by an N-terminal X₁ and a C-terminal Y₁, wherein A₁ is an anionic amino acid selected from Asp (D) and Glu (E), and/or wherein A₄ is a polar neutral amino acid selected from the group consisting of Ser(S) and Thr (T), and/or wherein A₇ is an aromatic amino acid selected from Phe (F), Tyr (Y), and Trp (W), and/or wherein A₉ is an aliphatic amino acid selected from the group consisting of Gly (G), Ala (A), Val (V), Leu (L), Ile (I), and Pro (P).

In other aspects, the first cysteine pair motif is a C₃-3 AA-C₄ motif having in contiguous sequence from its N-terminal end three amino acids (A₁₁, A₁₂, and A₁₃) flanked by an N-terminal C₃ and a C-terminal C₄ wherein A₁₁ is an aliphatic amino acid selected from Gly (G), Ala (A), Val (V), Leu (L), Ile (I), and Pro (P) or a cationic amino acid selected from His (H), Arg (R), and Lys (K) and/or wherein A₁₃ is an aliphatic amino acid selected from the group consisting of Gly (G), Ala (A), Val (V), Leu (L), Ile (I), and Pro (P) or a polar neutral amino acid selected from the group consisting of Ser(S) and Thr (T).

In yet other aspects, the second cysteine pair motif is a C₅-5-6AA-C₆ motif having in contiguous sequence from its N-terminal end five or six amino acids (A₁₄, A₁₅, A₁₆, A₁₇, A₁₈ and, optionally, A₁₉) flanked by an N-terminal C₅ and a C-terminal C₆, wherein A₁₄ is a cationic amino acid selected from His (H), Arg (R), and Lys (K) or an aliphatic amino acid selected from Gly (G), Ala (A), Val (V), Leu (L), Ile (I), and Pro (P) or a polar neutral amino acid selected from Ser(S) and Thr (T), and/or wherein A₁₆ is an aliphatic amino acid selected from the group consisting of Gly (G), Ala (A), Val (V), Leu (L), Ile (I), and Pro (P) or an aromatic amino acid selected from the group consisting of Phe (F), Tyr (Y), and Trp (W), and/or wherein A₁₈ is a cationic amino acid selected from the group consisting of His (H), Arg (R), and Lys (K).

In further aspects, the second amino acid pair motif is a X₂-3AA-Y₂ motif having in contiguous sequence from its N-terminal end three amino acids (A₂₀, A₂₁, and A₂₂) flanked by an N-terminal X₂ and a C-terminal Y₁, wherein A₂₀ is a polar neutral amino acid selected from the group consisting of Ser(S) and Thr (T), and/or wherein A₂₁ is a cationic amino acid selected from His (H), Arg (R), and Lys (K), and/or wherein A₂₂ is an aliphatic amino acid selected from the group consisting of Gly (G), Ala (A), Val (V), Leu (L), Ile (I), and Pro (P).

In still further aspects, IS1 includes five amino acids, and/or IS2 includes from nine to eleven amino acids, and/or IS3 includes one amino acid that is an aliphatic amino acid selected from the group consisting of Gly (G), Ala (A), Val (V), Leu (L), Ile (I), and Pro (P) or an aromatic amino acid selected from the group consisting of Phe (F), Tyr (Y), and Trp (W).

In still further aspects, the Defensin peptide includes a Defensin gamma core peptide sequence GXC₅X3-9C₆ (SEQ ID NO: 455) or variant gamma core peptide sequence GXC₅X₃-10C₆ (SEQ ID NO: 456).

Within other aspects of these compositions, the Defensin peptide is a cationic Defensin peptide that is a native Defensin peptide or a synthetic variant of a native Defensin peptide having one or more amino acid insertions, deletions, and/or substitutions in a native Defensin peptide and, optionally, wherein the substitutions in a native Defensin peptide comprise at least one of a substitution of a hydrophobic amino acid with a hydrophobic amino acid, a cationic amino acid with a cationic amino acid, or an anionic amino acid with a cationic or anionic amino acid, and/or wherein the amino acid sequence of the Defensin peptide is at least 70%, 75%, 80%, 85%, 90%, 95%, 97%, or 99% sequence identity to any one of SEQ ID NO: 1 to 454 or SEQ ID NO: 459 to SEQ ID NO: 472

In further aspects, the cysteine residues of DEF_PFV1 form a first set of disulfide bonds and DEF_PFV1 thereby adopts a first conformation that exhibits greater antimicrobial activity as compared to a Defensin peptide folding variant 2 (DEF_PFV2) that has the same amino acid sequence as DEF_PFV1 but forms a distinct second set of disulfide bonds between cysteine residues and adopts a distinct second conformation. Defensin peptides according to these embodiments have an isoelectric point of between 8.0 and 12.0 and/or a DEF_PFV1 exhibits greater protease resistance or greater protease sensitivity than the corresponding DEF_PFV2. Typically, these compositions are substantially free of DEF_PFV2.

In other aspects, the Defensin peptide includes a Defensin gamma core peptide sequence GXC₅X3-9C₆ (SEQ ID NO: 455) or a variant gamma core peptide sequence GXC₅X₃-10C₆ (SEQ ID NO: 456), GXC₅X3-9 (F/W/Y) (SEQ ID NO: 457), or GXC₅X3-10 (F/W/Y) (SEQ ID NO: 458) and at least two or three additional cysteine residues selected from the group consisting of cysteine residues corresponding to C₁, C₂, C₃, C₄, C₇, and C₈ of a reference Defensin peptide. In certain embodiments, the Defensin peptide comprises a C-terminal fragment of a defensin peptide comprising a Defensin gamma core peptide sequence GXC₅X3-9C₆ (SEQ ID NO: 455) or variant gamma core peptide sequence GXC₅X3-10C₆ (SEQ ID NO: 456) and cysteine residues corresponding to C₇ and C₈ of a reference Defensin peptide.

In related aspects, the cationic Defensin peptide includes at least four additional cysteine residues selected from cysteines corresponding to C₁, C₂, C₃, C₄, C₇, and Ca of a reference Defensin peptide, and/or wherein one to four of the cysteine residues corresponding to C₁, C₂, C₃, C₄, C₇, and C₈ of a reference Defensin peptide are substituted with a different amino acid or are deleted, and/or wherein one or more cysteines is substituted with a Ser(S) or Thr (T) residue.

In other related aspects, at least one of the cysteine residues corresponding to C₁, C₂, C₃, C₄, C₇, and C₈ of the reference Defensin peptide is substituted with a different amino acid, at least one of the of the cysteine residues corresponding to C₁, C₂, C₃, C₄, C₅, C₆, C₇, and C₈ of the reference Defensin peptide is deleted, wherein at least two of the cysteine residues corresponding to C₁, C₂, C₃, C₄, C₇, and C₈ of the reference Defensin peptide are retained, and/or the Defensin peptide comprises cysteine residues corresponding to C₅, C₆, C₇, and C₈ of a reference Defensin peptide.

In other aspects, the Defensin peptide includes a C₅-5-6AA-C₆ motif having in contiguous sequence from its N-terminal end three amino acids (A₁₄, A₁₅, A₁₆, A₁₇, A₁₈ and, optionally, A₁₉) flanked by an N-terminal C₅ and a C-terminal C₆, wherein: (i) A₁₄ is a cationic amino acid selected from His (H), Arg (R), and Lys (K) or an aliphatic amino acid selected from Gly (G), Ala (A), Val (V), Leu (L), Ile (I), and Pro (P) or a polar neutral amino acid selected from the group consisting of Ser(S) and Thr (T); wherein A₁₆ is an aliphatic amino acid selected from Gly (G), Ala (A), Val (V), Leu (L), Ile (I), and Pro (P) or an aromatic amino acid selected from Phe (F), Tyr (Y), and Trp (W); and/or (iii) wherein A₁₈ is a cationic amino acid selected from His (H), Arg (R), and Lys (K).

In further aspects, the Defensin peptide includes a C₇-3AA-C₈ motif having in contiguous sequence from its N-terminal end three amino acids (A₂₀, A₂₁, and A₂₂) flanked by an N-terminal C₇ and a C-terminal C₈, wherein (i) A₂₀ is a polar neutral amino acid selected from Ser(S) and Thr (T); (ii) A₂₁ is a cationic amino acid selected from His (H), Arg (R), and Lys (K); and/or (iii) A₂₂ is an aliphatic amino acid selected from Gly (G), Ala (A), Val (V), Leu (L), Ile (I), and Pro (P).

Defensin peptides according to these embodiments can comprise an amino acid sequence having at least 70%, 75%, 80%, 85%, 90%, 95%, 97%, or 99% sequence identity to any one of the exemplary Defensin peptides that are presented in Table 2. Depending upon the precise application contemplated, compositions according to these embodiments can comprise an agriculturally, pharmaceutically, or veterinary practicable carrier, diluent, or excipient.

DEF_PFV1 can be separated from mixtures comprising the desired DEF_PFV1 and one or more undesired DEF_PFV2 to provide compositions that are enriched for the desired DEF_PFV1. Compositions comprising the desired DEF_PFV1 typically exhibit improved unit activity (e.g., units of anti-microbial activity per micrograms or micromoles of Defensin protein) relative to compositions comprising a mixture of the desired DEF_PFV1 and one or more reduced activity DEF_PFV2. Depending upon the precise application contemplated for the DEF_PFV1 compositions, it can be desirable to employ a DEF_PFV1 having an increased protease resistance or an increased protease sensitivity (e.g., to a serine protease including trypsin) in comparison to the undesired DEF_PFV2.

Compositions comprising a desired DEF_PFV1 can be substantially free of an undesired DEF_PFV2. In certain embodiments, such compositions can comprise less than about 20%, 10%, 5%, 2%, 1%, or 0.5% by weight of an undesired DEF_PFV2 and respectively at least about 80%, 90%, 95%, 98%, 99%, or 99.5% by weight of a desired DEF_PFV1. In certain embodiments, the composition comprising the desired DEF_PFV1 which is substantially free of the undesired DEF_PFV2 can comprise less than about 20%, 10%, 5%, 2%, 1%, or 0.5% of the combined total of the desired DEF_PFV1 and undesired DEF_PFV2 by weight in the composition as the undesired DEF_PFV2. In certain embodiments, the composition comprising the desired DEF_PFV1 which is substantially free of the undesired DEF_PFV2 can comprise at least about 80%, 90%, 95%, 98%, 99%, or 99.5% of the total defensin peptide in the composition by weight (e.g., total defensin peptide in the form of the desired DEF_PFV and one or more undesired DEF_PFV1 and one or more undesired DEF_PFV2) as the desired DEF_PFV1. In certain embodiments, less than 20%, 10%, 5%, 2%, 1%, or 0.5% of the total defensin peptide in the composition by weight (e.g., total defensin peptide in the form of the desired DEF_PFV and one or more undesired DEF_PFV1 and one or more undesired DEF_PFV2) is the undesired DEF PFV2 and respectively at least about 80%, 90%, 95%, 98%, 99%, or 99.5% of the total defensin peptide in the composition by weight is the desired DEF_PFV1.

Without seeking to be limited by theory, it is contemplated that compositions comprising or enriched for a desired DEF_PFV1 that exhibit an increased protease resistance relative to an undesired DEF_PFV2 can be useful in certain of the methods disclosed herein where an extended shelf-life or half-life of the DEF_PFV1 is desirable. Alternatively, it is contemplated that compositions comprising or enriched for a desired DEF_PFV1 that exhibit an increased protease sensitivity relative to an undesired DEF_PFV2 can be useful in such methods where a decreased half-life of the DEF_PFV1 is desirable.

Suitable Defensin peptides for generating DEF_PFV1 peptides can include Defensin peptides comprising an amino acid sequence as set forth in Table 1 or Table 2. Variants of these Defensin peptides are contemplated wherein, for example, one or more of the hydrophobic, basic, and/or acidic amino acid residue(s) is substituted with an alternative hydrophobic, basic, and/or acidic amino acid residues. In embodiments provided herein, the Defensin peptide variants comprise an amino acid sequence having at least 60%, 70%, 80%, 85%, 90%, 92%, 95%, 97%, 98%, 99%, or 100% sequence identity across the entire length of one or more Defensin peptide(s) presented in Tables 1 and 2 and/or as set forth SEQ ID NO: 1 to 454 or 459 to 472.

Conserved cysteine residues in Defensin peptides can be identified as C₁, C₂, C₃, C₄, C₅, C₆, C₇, and C₈ as depicted in Table 1. For Defensin peptides, the conserved cysteine residue closest to the amino terminus is C₁ and the cysteine residue closest to the carboxy terminus is C₈ as depicted in Table 1.

Within certain aspects of these embodiments, Defensin peptides that can adopt alternative disulfide bonding pairs and that constitute Defensin peptide folding variants can have a net positive charge at neutral pH. For example, certain Defensin peptides have a net positive charge at neutral pH of at least +4, +5, +6, +7, +8, +9, or +10 to +12, +13, +14, or +15 or, alternatively, of at least +5, +6, +7, +8, +9, or +10. It will be understood that such net positive charges in Defensin peptides can be achieved by methods that include: (i) maintaining cationic (basic) amino acid residues found in the Defensin peptides (including Defensin peptides presented in Tables 1 and 2, respectively) or substituting such residues with another cationic amino acid residue; (ii) substituting anionic or polar amino acid residues found in Defensin peptides (including Defensin peptides presented in Tables 1 and 2, respectively) with a basic amino acid residue; or a combination of (i) and (ii). Such net positive charges in Defensin peptides can be achieved by preferentially selecting or substituting a cationic amino acid residue at variable positions in the Defensin peptide that correspond to a variable position.

Within other aspects of these embodiments, Defensin peptides that can adopt alternative disulfide bonding pairs and that constitute Defensin peptide folding variants can include a significant percentage of hydrophobic amino acid residues. For example, Defensin peptides can comprise at least about 25%, 26%, 28% 30%, 32%, 34%, or 36% to 37%, 38%, 40%, 42%, or 45% hydrophobic amino acid residues or at least about 25%, 26%, 28% 30%, 32%, 34%, 36%, 37%, or 38% hydrophobic amino acid residues. It will be understood that such percentages of hydrophobic amino acids in Defensin peptides can be achieved by methods that include: (i) maintaining hydrophobic amino acid residues found in Defensin peptides (including Defensin peptides presented in Tables 1 and 2, respectively) or substituting such residues with another hydrophobic amino acid residue or neutral polar amino acid residue; (ii) substituting polar amino acid residues found in Defensin peptides (including Defensin peptides presented in Tables 1 and 2, respectively) with a hydrophobic amino acid residue; (iii) substituting neutral polar amino acids for hydrophobic amino acids; or a combination of (i), (ii), and (iii)). Such percentages of hydrophobic amino acids in Defensin peptides can be achieved by preferentially selecting or substituting a hydrophobic amino acid residue at variable positions in the Defensin peptide that correspond to a variable position.

Within further aspects of these embodiments, Defensin peptides include four, five, six, or seven of the eight conserved C₁, C₂, C₃, C₄, C₅, C₆, C₇, and C₈ cysteine residues set forth in Table 1. Defensin peptide folding variant 1 (DEF_PFV1) can form a first set of disulfide bonds that comprise a C₁-C₈ disulfide bond, a C₂-C₅ disulfide bond, a C₃-C₆ disulfide bond, and a C₄-C₇ disulfide bond, and/or wherein Defensin peptide folding variant 2 (DEF_PFV2) forms a second set of disulfide bonds other than a C₁-C₈ disulfide bond, a C₂-C₅ disulfide bond, a C₃-C₆ disulfide bond, and a C₄-C₇ disulfide bond.

Defensin peptides can also comprise four, five, six, or seven of the eight conserved C₁, C₂, C₃, C₄, C₅, C₆, C₇, and C₈ cysteine residues. One or more of the cysteinyl residues in Defensin peptide can, for example, be substituted with a distinct amino acid residue or deleted to provide an Defensin peptide with only four, five, six, or seven of the eight conserved C₁, C₂, C₃, C₄, C₅, C₆, C₇, and C₈ cysteine residues. Alternatively, one or more of the conserved C₁, C₂, C₃, C₄, C₅, C₆, C₇, and C₈ cysteine residues in a Defensin peptide can be substituted with another amino acid residue including a glycine, serine, threonine, cysteine, cystine, tyrosine, asparagine, or glutamine residue to provide an Defensin peptide with only four, five, six, or seven of the eight conserved C₁, C₂, C₃, C₄, C₅, C₆, C₇, and C₈ cysteine residues. For example, it can be desirable to substitute one or two of the conserved cysteine residues with a serine residue to provide an Defensin peptide with only four, five, six, or seven of the eight conserved C₁, C₂, C₃, C₄, C₅, C₆, C₇, and C₈ cysteine residues. Such Defensin peptides having only four, five, six, or seven of the eight conserved cysteine residues can comprise a deletion or a substitution of the C₁, C₂, C₃, and/or C₄ cysteine residues and will thus comprise the C₅, C₆, C₇, and C₈ cysteine residues. Alternatively, a Defensin peptide with only four, five, six, or seven of the eight conserved cysteine residues can comprise a deletion or a substitution of the C₁ and C₂ cysteine residues in a Defensin peptide and thus comprise the C₃, C₄, C₅, C₆, C₇, and C₈ cysteine residues.

Alternatively, Defensin peptides can comprise a Defensin gamma core peptide sequence GXC₅X3-9C₆ (SEQ ID NO: 455) or variant gamma core peptide sequence GXC₅X₃-10C₆ (SEQ ID NO: 456) and at least two additional cysteine residues selected from the group consisting of cysteine residues corresponding to C₁, C₂, C₃, C₄, C₇, and C₈ of a reference Defensin peptide. A cationic Defensin peptide variant can include at least four additional cysteine residues selected from cysteines corresponding to C₁, C₂, C₃, C₄, C₅, C₆, C₇, and C₈ of a reference Defensin peptide and/or one to four of the cysteine residues corresponding to C₁, C₂, C₃, C₄, C₇, and C₈ of a reference Defensin peptide can be substituted with a different amino acid or deleted. One or more cysteines can be substituted with a Ser(S) or Thr (T) residue.

Defensin peptide variants can also include substitution of at least one of the cysteine residues corresponding to C₁, C₂, C₃, C₄, C₇, and C₈ of the reference Defensin peptide with a different amino acid and/or at least one of the of the cysteine residues corresponding to C₁, C₂, C₃, C₄, C₅, C₆, C₇, and C₈ of the reference Defensin peptide can be deleted, wherein typically at least two of the cysteine residues corresponding to C₁, C₂, C₃, C₄, C₇, and C₈ of the reference Defensin peptide are retained. For example, Defensin peptide variants can comprises cysteine residues corresponding to C₅, C₆, C₇, and C₈ of a reference Defensin peptide and substitute C₁, C₂, C₃, and C₄ with another amino acid, such as a Ser(S) or Thr (T) residue, or Defensin peptide variants can include a deletion of one or more of C₁, C₂, C₃, and C₄.

Suitable Defensin peptides for use in preparing compositions according to these embodiments can comprise an amino acid sequence that is at least 60%, 70%, 75%, 80%, 85%, 90%, 95%, 98%, 99%, or 100% identical to one or more of the Defensin peptide sequences presented in Table 1 or one or more of the Defensin peptide sequences presented in Table 2 wherein one or more of the hydrophobic, basic, and/or acidic amino acid residues are substituted with other hydrophobic, basic, and/or acidic amino acid residues, respectively.

One or more amino acids in any of the Defensin peptides presented in Table 1 and Table 2 or in a variant Defensin peptide can be substituted with other amino acid(s), the charge and polarity of which is similar to that of the original amino acid, i.e., a conservative amino acid substitution. Substitutes for an amino acid within the Defensin peptide sequence can be selected from other members of the class to which the originally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (anionic; negatively charged) amino acids such as aspartic acid and glutamic acid; (2) basic (cationic; positively charged) amino acids such as arginine, histidine, and lysine; (3) neutral polar amino acids such as glycine, serine, threonine, cysteine, cystine, tyrosine, asparagine, and glutamine; (4) neutral nonpolar (hydrophobic) amino acids such as alanine, leucine, isoleucine, valine, proline, phenylalanine, tryptophan, and methionine. Conservative amino acid changes within Defensin peptide sequences can be made by substituting one amino acid within one of these groups with another amino acid within the same group.

Biologically functional equivalents of Defensin peptides can have 10 or fewer conservative amino acid changes, seven or fewer conservative amino acid changes, or five, four, three, two, or one conservative amino acid changes. The encoding nucleotide sequence (e.g., gene, plasmid DNA, cDNA, or synthetic DNA) will thus have corresponding base substitutions, permitting it to encode biologically functional equivalent forms of the Defensin peptides.

Certain semi-conservative substitutions in Defensin peptides including: (i) the substitution of a neutral polar amino acid residue with a neutral nonpolar (hydrophobic) amino acid residue; or (ii) the substitution of a neutral nonpolar (hydrophobic) amino acid residue with a neutral polar amino acid residue are also provided. In particular, semi-conservative substitutions of a neutral polar tyrosine residue with a hydrophobic amino acid residue are provided. Semi-conservative substitutions of a hydrophobic amino acid residue with tyrosine residue are also provided. Biologically functional equivalents of Defensin peptides can have 10 or fewer semi-conservative amino acid changes, seven or fewer semi-conservative amino acid changes, or five, four, three, two, or one semi-conservative amino acid changes.

Functional fragments of any of the Defensin peptides or Defensin peptide variants disclosed herein can include amino terminal deletions, carboxy terminal deletions, internal deletions, or any combination thereof that leave intact the conserved cysteine residues of a Defensin or contain at least four, five, six, or seven of the eight conserved C₁, C₂, C₃, C₄, C₅, C₆, C₇, and C₈ cysteine residues of a reference Defensin peptide. Functional fragments can contain at least one, two, three, four, five, six, or seven or more amino acid residue deletions from the amino terminus, the carboxy terminus, an internal region, or any combination thereof. For example, antimicrobial fragments of a Defensin peptide can comprise at least about 10, 14, 15, 18, or 20 to about 22, 24, 25, 26, 27, or 28 amino acid residues of the C-terminus of the Defensin peptide. Chimeric Defensin peptides comprising portions of any of the Defensin disclosed herein or comprising variants or fragments of those Defensin can also be used either alone or in the Defensin proteins provided herein.

### Methods for Making, Testing, and Using Antimicrobial DEF_PFV Compositions

Within related embodiments, the present disclosure provides methods for making, testing, and using antimicrobial DEF_PFV compositions comprising a cationic antimicrobial Defensin (DEF) peptide folding variant (PFV), which compositions (1) include an Defensin peptide folding variant 1 (DEF_PFV1) having a desired antimicrobial activity and/or resistance/sensitivity to proteolysis and (2) exclude an Defensin peptide folding variant 2 (DEF_PFV2) having the same or a decreased antimicrobial activity and/or an undesired resistance or sensitivity to proteolysis.

DEF_PFV1 can comprise an amino acid sequence at least 50%, 60%, 70%, 75%, 80%, 85%, 90%, 95%, 98%, 99%, or 100% identical to an amino acid sequence presented in Table 1 or Table 2 or a variant of the amino acid sequence an amino acid sequence presented in Table 1 or Table 2, wherein one or more of the hydrophobic, basic, and/or acidic amino acid residues are substituted with hydrophobic, basic, and/or acidic amino acid residues, respectively; a variant of the amino acid sequence presented in Table 1 or Table 2, wherein one or more neutral polar amino (e.g., tyrosine) acid residues is substituted with a hydrophobic amino acid residue; and/or wherein one or more hydrophobic amino acid residues is substituted with a neutral polar amino acid residue (e.g., tyrosine); functional fragments thereof, and chimeras thereof.

Defensin peptides or proteins can synthesized de novo from an Defensin peptide sequence disclosed herein or can be expressed from a nucleotide sequence encoding a Defensin peptide or protein. The sequence of the peptide or protein-encoding nucleotide sequence can be deduced from the Defensin peptide sequence by reference to the genetic code. Computer programs such as “BackTranslate” (GCG™ Package, Acclerys, Inc. San Diego, CA) can be used to convert a peptide sequence to the corresponding nucleotide sequence that encodes the peptide.

Expression of Defensin peptides in yeast and filamentous fungi to produce DEF_PFV1 is specifically contemplated herein. The construction of expression vectors for production of heterologous proteins in various yeast genera is well established. In general, such expression vectors typically comprise a promoter that is operably linked to a sequence of interest which is operably linked to a polyadenylation or terminator region. Examples of yeast genera that have been used to successfully express heterologous genes include Candida, Kluveromyces, Hansuela, Pichia, Saccharomyces, Schizosaccharomyces, and Yarrowia. A general description of expression vectors and transformation systems for Saccharomyces is found in Kingsman et al (1985) Biotechnol Genet Eng Rev. 3:377-416. Expression vectors and transformation systems useful for yeasts other than Saccharomyces are described in Reiser et al (1990) Adv Biochem Eng Biotechnol. 43:75-102.

Other examples of fungal systems which can be adapted for use in expressing Defensin peptides include filamentous fungal systems such as Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes and Trichoderma systems (e.g. U.S. Pat. Nos. 11,046,736 and 11,180,767, both incorporated herein by reference in their entireties). Other systems which can be adapted for use in expressing Defensin peptides include Chrysosporium lucknowense systems (e.g., U.S. Pat. Nos. 8,871,493, and 9,175,296, incorporated herein by reference in its entirety).

Expression of Defensin peptides in bacterial cells including Escherichia sp. (e.g., E. coli) to produce Defensin PFV is also specifically contemplated herein. Systems for expressing proteins which comprise disulfide bonds can be adapted for expression of the Defensin peptides in E. coli include those disclosed in U.S. Patent Publication No. US 2020/0172915, which is incorporated herein by reference in its entirety, and in Berkmen, M. Protein Expr Purif. 2012; 82 (1): 240-51. doi: 10.1016/j.pep.2011.10.009. Other systems useful for expression of proteins which comprise disulfide bonds can be adapted for expression of the Defensin peptides in E. coli include those disclosed in Kuddus et al., (2017) Biotechnol Prog 233:1520-1528. doi: 10.1002/btpr.Protein Science 2508; Kiedzierska et al. (2008) Protein Expr Purif 60, 82-88; Chang et al., (2015) Amino Acids 47, 579-587; Buchko et al. (2018) (2018) Protein Science 27, 1611-1623; and Marques et al., (2008) J Appl Microbiol 106, 1640-1648; Pazgier, M., Lubkowski, J. (2006) Protein Expr Pur 49, 1-8.

In general, the promoter and polyadenylation region are selected based on their operability in a given bacterial, yeast, or fungal host. For example, the AOX1 or AOX2 promoters of Pichia can be used in conjunction with the AOX1, AOX2, p40, or p76 polyadenylation sequences of Pichia to express a heterologous protein such as an Defensin peptide. Both the AOX1 and AOX2 promoters are particularly useful in Pichia as both promoters provide for abundant expression of the linked heterologous gene when induced by addition of methanol to the growth medium.

The use of these Pichia promoters and polyadenylation sequences is described in U.S. Pat. No. 4,855,231, which is expressly incorporated herein by reference in its entirety. Similarly, the Hansuela MOX, DHAS, or FMDH promoters can be used to express heterologous proteins such as Defensin in Hansuela. The MOX, DHAS, or FMDH promoters are particularly useful in Hansuela as these promoters provide for abundant expression of the linked heterologous gene when induced by addition of methanol to the growth medium. The use of the MOX and DHAS promoters in Hansuela is described in U.S. Pat. No. 5,741,672, while the use of the FMDH promoter in Hansuela is described in U.S. Pat. No. 5,389,525, each of which is expressly incorporated herein by reference in its entirety. For Kluveromyces, a Lactase promoter and polyadenylation sequence can be used to express heterologous genes such as Defensin. Expression of heterologous genes that are operably linked to the Lactase promoter and polyadenylation sequence is achieved by growing Kluveromyces in the presence of galactose. The use of the Lactase promoter and polyadenylation sequences in Kluveromyces is described in U.S. Pat. No. 6,602,682, which is expressly incorporated herein by reference in its entirety.

Yeast, bacterial, or fungal expression vectors that provide for secretion of heterologous proteins such as Defensin into the growth medium by transformed yeast or fungi are also contemplated. Secretion of the mature Defensin peptide is typically achieved by operable linkage of a signal peptide sequence or a signal peptide and propeptide sequence to the mature Defensin protein- or peptide-encoding sequence. Examples of useful signal peptides for secretion of heterologous proteins in yeast include but are not limited to an alpha-factor signal peptide, an invertase signal peptide, and a PHOI signal peptide, all of which are derived from yeast. The alpha-factor signal peptide is typically derived from Saccharomyces, Kluveromyces, or Candida, while the PHOl signal peptide is derived from Pichia.

A particularly useful signal peptide sequence or signal peptide and propeptide sequence for secretion of proteins in yeast is derived from the S. cerevisiae alpha-factor, and is described in U.S. Pat. Nos. 4,546,082, 4,588,684, 4,870,008, and 5,602,034, each of which is expressly incorporated herein by reference in its entirety. The S. cerevisiae alpha-factor signal peptide and propeptide sequence consist of amino acids 1-83 of the primary, unprocessed translation product of the S. cerevisiae alpha mating factor gene (GenBank Accession Number: P01149). In certain embodiments, the signal peptide sequence of the alpha-mating factor comprising amino acids 1 to about 19 to 23 of the alpha-mating factor proprotein can be directly linked to the N-terminus of the mature Defensin protein to provide for secretion of mature Defensin protein. In this case, the signal peptide is cleaved from the mature Defensin protein in the course of the secretion process. Alternatively, the signal peptide and propeptide of the alpha mating factor can be operably linked to the mature Defensin encoding sequence via a cleavage site sequence. This cleavage site sequence can comprise a variety of sequences that provide for proteolytic processing of the leader sequence and gene of interest.

In the native S. cerevisiae alpha mating factor gene the s cleavage site sequence corresponds to amino acid residues 84-89 and is represented by the sequence Lys84-Arg85-Glu86-Ala87-Glu88-Ala 89 (SEQ ID NO: 473). The sequence Lys-Arg corresponds to a KEX2 protease recognition site while the Glu-Ala-Glu-Ala sequence corresponds to a duplicated dipeptidylaminopeptidase or STE13 recognition site. In certain embodiments, a DNA fragment encoding the 89 amino acid S. cerevisiae alpha factor signal, propeptide coding region, and entire native spacer coding region (i.e., the N-terminal 89 amino acid residues of the alpha mating factor precursor protein containing both the Lys-Arg KEX2 protease cleavage site at residues 84 and 85 as well as the Glu-Ala-Glu-Ala dipeptidylaminopeptidase or STE13 recognition site at residues 86-89) is operably linked to the sequence encoding the mature Defensin protein.

When the N-terminal 89 amino acids of the alpha mating factor precursor protein are fused to the N-terminus of a heterologous protein such as Defensin, the propeptide sequence is typically dissociated from the heterologous protein via the cleavage by endogenous yeast proteases at either the KEX2 or STE13 recognition sites. In other embodiments, a DNA fragment encoding the smaller 85 amino acid Saccharomyces cerevisiae alpha factor signal peptide, propeptide, and KEX2 spacer element (i.e., the N-terminal 85 amino acid residues of the alpha mating factor precursor protein containing just the Lys-Arg KEX2 protease cleavage site at residues 84 and 85) is operably linked to the sequence encoding the mature Defensin protein. When the N-terminal 85 amino acids of the alpha mating factor precursor protein are fused to the N-terminus of a heterologous protein such as Defensin, the propeptide sequence is typically dissociated from the heterologous protein via cleavage by endogenous yeast proteases at the KEX2 recognition site. The Defensin protein can thus be expressed without the glu-ala repeats.

To obtain transformed yeast that express Defensin peptides, the yeast Defensin expression cassettes (e.g., yeast promoter, yeast signal peptide encoding sequence, mature Defensin protein sequence, and polyadenylation sequence) are typically combined with other sequences that provide for selection of transformed yeast. Examples of useful selectable marker genes include genes encoding a ADE protein, a HIS5 protein, a HIS4 protein, a LEU2 protein, a URA3 protein, ARG4 protein, a TRP1 protein, a LYS2 protein, a protein conferring resistance to a bleomycin or phleomycin antibiotic, a protein conferring resistance to chloramphenicol, a protein conferring resistance to G418 or geneticin, a protein conferring resistance to hygromycin, a protein conferring resistance to methotrexate, an a AR04-OFP protein, and a FZF1-4 protein. Similar electable marker cassettes that confer resistance to antibiotics or rescue auxotrophic traits can be used in bacterial or fungal systems.

DNA molecules comprising the yeast Defensin expression cassettes and selectable marker genes are introduced into yeast cells by techniques such as transfection into yeast spheroplasts or electroporation. In certain embodiments, the DNA molecules comprising the yeast Defensin expression cassettes and selectable marker genes are introduced as linear DNA fragments that are integrated into the genome of the transformed yeast host cell. Integration can occur either at random sites in the yeast host cell genome or at specific sites in the yeast host cell genome. Integration at specific sites in the yeast host cell genome is typically accomplished by homologous recombination between sequences contained in the expression vector and sequences in the yeast host cell genome.

Homologous recombination is typically accomplished by linearizing the expression vector within the homologous sequence (for example, within the AOX1 promoter sequence of a Pichia expression vector when integrating the expression vector into the endogenous AOX1 gene in the Pichia host cell). In other embodiments, the yeast expression cassettes can also comprise additional sequences such as autonomous replication sequences (ARS) that provide for the replication of DNA containing the expression cassette as an extrachromosomal (non-integrated) element. Such extra-chromosomal elements are typically maintained in yeast cells by continuous selection for the presence of the linked selectable marker gene. Yeast artificial chromosomes (YACs) containing sequences that provide for replication and mitotic transmission are another type of vector that can be used to maintain the DNA construct in a yeast host.

Yeast, bacterial, or fungal cells transformed with the yeast or fungal Defensin expression cassettes can be used to produce mixtures of Defensin PFV which can be used to obtain preparation enriched for a desired DEF_PFV. The desired DEF_PFV can be used as antimicrobial agents, to produce antimicrobial compositions that can be applied to plants or foodstuffs, or to produce antimicrobial compositions that can be administered to human or non-human subjects. Methods of producing mixtures of DEF_PFV can typically first comprise the step of culturing yeast, bacterial, or fungal cells transformed with Defensin expression cassettes under conditions wherein the yeast cells express a mature Defensin molecule.

In general, the conditions where the yeast bacterial, or fungal cells express the mature Defensin molecules are conditions that allow for or specifically induce expression of the yeast promoter that is operably linked to the Defensin coding sequence in the yeast expression cassette. When the yeast is Pichia and the signal-peptide/MD gene is under the control of an AOX1 or AOX2 promoter, addition of methanol to the growth medium will provide for expression of mature Defensin protein. Similarly, when the yeast is Hansuela and the signal-peptide/MD gene is under the control of a MOX, DHAS, or FMDH promoter, addition of methanol to the growth medium will provide for expression of mature Defensin protein. Alternatively, when the yeast is Kluveromyces and the signal-peptide/De/5 gene is under the control of a Lactase promoter, addition of galactose to the growth medium will provide for expression of mature Defensin protein.

Once the transformed yeast, bacterial, or fungal culture has been incubated under culture conditions that provide for expression of mature Defensin peptide for a sufficient period of time, the desired Defensin molecule PFV can be separated from any undesired DEF_PFV isolated from the culture as a mixture. A sufficient period of time can be determined by periodically harvesting portions or aliquots of the culture and assaying for the presence of desired and/or undesired DEF_PFV. Analytical assays such as HPLC be used to monitor desired DEF_PFV and undesired DEF_PFV production. In certain embodiments, culture incubation periods can be optimized for production of the desired DEF_PFV such that the culture is enriched for the desired DEF_PFV.

Methods according to these embodiments comprise (a) separating a fraction comprising a Defensin peptide folding variant 1 (DEF_PFV1) from one or more fractions comprising Defensin peptide folding variant 2 (DEF_PFV2), or DEF_PFV2 peptide fragments thereof, from a mixture comprising DEF_PFV1 and DEF_PFV2, or DEF_PFV2 peptide fragments thereof; and (b) combining said fraction comprising DEF_PFV1, or a preparation further purified from said DEF_PFV1 fraction, with an agriculturally, pharmaceutically, or veterinary practicable carrier, diluent, or excipient, thereby making the composition.

Mixtures comprising DEF_PFV1 and DEF_PFV2, or DEF_PFV2 peptide fragments thereof, can be prepared by (a) culturing in fermentation broth microorganisms expressing a recombinant polynucleotide comprising a transcriptional promoter operably linked to a polynucleotide encoding a signal peptide that is in frame with and upstream of a polynucleotide encoding DEF_PFV1, or a variant thereof, (b) separating (e.g., by size-exclusion chromatography) the microorganisms from the fermentation broth, which comprises the mixture, (c) disrupting the microorganisms to obtain a cell lysate, and (d) separating insoluble debris in the cell lysate from an aqueous fraction comprising said mixture.

Isolation of the desired DEF_PFV from the culture can be partial or complete. For Defensin expression vectors where a yeast, bacterial, or fungal signal peptide is operably linked to the sequence encoding the mature Defensin protein, a mixture comprising the desired DEF_PFV and any DEF_PFV or other undesired DEF_PFV (e.g., increased or decreased protease resistance) can be recovered from the yeast cell culture medium, bacterial periplasm, or fungal cell culture medium. In certain embodiments, yeast or fungal cell culture medium that contains the mature Defensin protein in the form of both a desired DEF_PFV and an undesired DEF_PFV can be separated from the yeast or fungal cells by centrifugation or filtration, thus providing a composition comprising a mixture of the desired and undesired DEF_PFV. In certain embodiments, preparations of bacterial periplasmic proteins that contains the mature Defensin protein in the form of both a desired DEF_PFV and an undesired DEF_PFV can be obtained by methods including physical force, pressure, osmotic shock, or chemical (e.g., EDTA+heat or detergent) treatment, thus providing a composition comprising a mixture of the desired and undesired DEF_PFV.

Such methods for isolation of bacterial periplasmic proteins can be adapted from methods disclosed for isolation of other periplasmic proteins (e.g., as in Schimek et al. (2020) Biotechnology progress, 36 (5), e2999. doi.org/10.1002/btpr.2999). Yeast or fungal cell culture medium or preparations of bacterial periplasmic proteins that contain the mature Defensin protein can be further processed by any combination of dialysis and/or concentration techniques (e.g., precipitation, lyophilization, filtration) to produce a composition containing one or more DEF_PFV (e.g. a mixture of the desired and undesired DEF_PFV). In certain embodiments, filtration size-exclusion membranes can be used to enrich for a fraction containing a mixture of desired and undesired DEF_PFV.

Combinations of various isolation techniques can also be employed to produce a mixture of DEF_PFV. For example, the cell culture medium can be separated from the cells by centrifugation and dialyzed or adjusted. In certain embodiments, a buffer for dialysis or adjustment is a 25 mM sodium acetate buffer at about pH 4.5-pH 6.0. This dialysate is then subjected to ion-exchange chromatography. For example, a cation-exchange resin such as CM-Sephadex C-25 equilibrated with a 25 mM sodium acetate buffer at about pH 6.0 can be used. Defensin protein bound to the cation exchange resin is washed and then eluted. For example, the aforementioned column is washed with 25 mM sodium acetate buffer at about pH 6.0 and subsequently eluted in 1M NaCl, 50 mM Tris, pH7.6. Fractions containing the Defensin protein are identified by an assay or by UV absorbance and then concentrated by a size-cutoff filtration membrane. The concentrated Defensin protein is then dialyzed to obtain an essentially or substantially pure Defensin protein in a buffer. Buffers include buffers such as 10 mM Tris, pH 7.6.

Production of compositions comprising or enriched for the desired DEF_PFV can be obtained by methods which provide for separation of the desired DEF_PFV from the undesired DEF_PFV(s). In certain embodiments, such methods can comprise standard protein separation techniques which enrich for the desired DEF_PFV based on different chemical properties besides mass (e.g., the hydrodynamic radius of different DEF_PFV and/or the charge characteristics of the different DEF_PFV). In certain embodiments, chromatographic techniques such as sedimentation, size exclusion chromatography (SEC), ion-exchange chromatography, and/or affinity chromatography can be used to produce a composition enriched for the desired DEF_PFV. Methods for separating proteins having different conformations by SEC which have been disclosed can be adapted for use in separating a desired from one or more undesired DEF_PFV(s) (La Verde et al., Bio Protoc. 2017 Apr. 20; 7 (8): e2230. doi: 10.21769/BioProtoc.2230).

In certain embodiments, the methods can provide for a composition comprising a desired DEF_PFV where the composition comprises less than 5%, 2%, 1%, 0.5%, 0.25%, or 0.1% of one or more undesirable DEF_PFV(s) by weight. In certain embodiments, the methods can provide for a composition comprising a desired DEF_PFV where less than 5%, 2%, 1%, or 0.5% of the combined total of the desired DEF_PFV and one or more undesired DEF_PFV(s) by weight in the composition is/are the undesired DEF_PFV(s). In certain embodiments, the methods can provide for a composition comprising a desired DEF_PFV where at least about 80%, 90%, 95%, 98%, 99%, or 99.5% of the total Defensin peptide by weight or molarity in the composition is the desired DEF_PFV.

In certain embodiments, the methods can provide a composition where less than 20%, 10%, 5%, 2%, 1%, or 0.5% of the total Defensin peptide in the composition by weight is undesired DEF_PFV or wherein at least about 80%, 90%, 95%, 98%, 99%, or 99.5% of the total Defensin peptide in the composition by weight is the desired DEF_PFV. Total Defensin peptide by weight in the composition can be determined by quantifying the combined total amount of desired and undesired DEF_PFV in the composition. Quantification of total Defensin peptides and/or an isolated DEF_PFV can be accomplished by a variety of methods including immunoassays (e.g., ELISAs, RIA where a purified Defensin peptide is used to establish a reference curve) or mass spectroscopy (Zhang et al. Methods Mol Biol. 2010; 673:211-222. doi: 10.1007/978-1-60761-842-3_13).

In certain embodiments, compositions enriched for desired DEF_PFV can be obtained by methods which exploit the relative sensitivity of a desired DEF_PFV to a protease (e.g., a serine protease such as trypsin). In embodiments where the desired DEF_PFV exhibits increased resistance to the protease (e.g., at a given protease concentration), mixtures comprising the desired and undesired DEF_PFV can be treated with the protease at a concentration which will cleave the undesired DEF_PFV yet leave the desired DEF_PFV intact. The intact DEF_PFV can then be separated from the resultant fragments of the Defensin peptide produced by cleavage of the undesired DEF_PFV by methods including size-exclusion membrane filtration, SEC, and/or IEC. Suitable proteases for such methods can include a trypsin family serine protease. In certain embodiments, the serine protease will cleave an undesired DEF_PFV at the carboxy terminus of an arginine or lysine residue in that undesired DEF_PFV under non-denaturing conditions. In certain embodiments, the trypsin family serine protease is a recombinant bovine, porcine, human, or microbial trypsin, optionally wherein the microbial trypsin is a Streptomycete trypsin or variant thereof. In certain embodiments, about 6.25, 10, or 12.5 to about 15, 20, or 25 BAEE units/mL of trypsin (e.g., Sigma-Aldrich T1426, St. Louis, MO) can be incubated with the DEF_PFV for about 1, 2, or 3 to about 4, 5, or 6 hours at 37° C. in 60 mM monobasic sodium phosphate buffer at pH 7.5 to cleave the undesired DEF_PFV yet leave the desired DEF_PFV intact. One BAEE (N_{α}-Benzoyl-L-arginine ethyl ester hydrochloride) trypsin substrate unit will produce a delta A253 (change in absorbance at 253 nm) of 0.001 per minute with BAEE as substrate at pH 7.6 at 25 deg C. in a reaction volume=3.2 ml (1 cm light path).

In some aspects of these methods a mixture comprising DEF_PFV1 and DEF_PFV2, or DEF_PFV2 peptide fragments is treated with a protease under non-denaturing conditions that are sufficient to produce DEF_PFV2 peptide fragments but insufficient to produce DEF_PFV1 peptide fragments. Suitable proteases include trypsin family serine proteases that cleave Defensin peptides at the carboxy terminus of an Arg (R) or Lys (K). Trypsin family serine proteases include recombinant bovine, porcine, human, or microbial trypsin, and, optionally, wherein the microbial trypsin is a Streptomycete trypsin or variant thereof.

A DEF_PFV1 according to the present embodiments can exhibit an increase in phospholipid binding in comparison to a corresponding a DEF_PFV2. A desired DEF_PFV1 provided herein can exhibit lower IC₅₀ values against one or more microbial pathogens, improved binding to phospholipids, or any combination thereof in comparison to either: (i) mixtures comprising the desired DEF_PFV1 and one or more corresponding undesired DEF_PFV2 or (ii) the DEF_PFV2 alone. The desired DEF_PFV1 can be identified and/or selected for lower IC₅₀ values against one or more microbial pathogens by selecting for DEF_PFV1 that provide for improved phospholipid binding in comparison to in comparison to either: (i) mixtures comprising the desired DEF_PFV1 and one or more corresponding undesired DEF_PFV2 or (ii) the DEF_PFV2 alone.

Suitable assays for determining improved phospholipid include protein-lipid overlay assays (e.g., Dowler et al., 2002, Sci STKE. 2002 Apr. 23; 2002 (129): 16), surface plasmon resonance assays (e.g., Baron and Pauron, 2014, Bio-protocol 4 (18): e1237), biotin capture lipid affinity assays (e.g., Davidson et al., 2006, Lipid Research, 47, 440-449), titration calorimetry assays (e.g., Miller and Cistola, 1993 Molecular and Cellular Biochemistry, 123 (1): 29-37), and the like.

Typically, the permeability of a microbial plasma membrane treated with a DEF_PFV1 as described herein can increase in comparison to permeability of a microbial plasma membrane treated with a corresponding a DEF_PFV2. Membrane permeability can be measured by a variety of techniques that include dye uptake. Convenient dye uptake assays that can be used to assess changes in in membrane permeability include assays for uptake of Hoechst 33342 (H0342), rhodamine 123, SYTOX™ Green, and the like. These dyes enter into microbial cells only if their plasma membrane has been permeabilized by a Defensin peptide, defensin, or other membrane-permeabilizing agent. Without seeking to be limited by theory, in certain embodiments it is believed that the Defensin protein comprising a Defensin peptide and another antifungal peptide joined by a spacer peptide can provide improved microbial inhibition by increasing the permeability of treated microbial membranes in comparison to microbial membranes treated with a Defensin peptide.

The use of microbial disease severity assays and colony formation assays in conjunction with expression assays to identify transgenic MsDefl-expressing potato plants that are resistant to Verticillium dahliae has been described (U.S. Pat. No. 6,916,970 and Gao et al., 2000, Nature Biotechnology 18, 1307-1310). It is similarly contemplated that a variety of DEF_PFV1 treated plants that combat or control microbial pathogens can be identified by scoring treated plants for resistance to microbial pathogens that infect those plants. Examples of DEF_PFV1-conferred microbial resistance that can be assayed by observing reductions in disease symptoms or reductions in microbial growth include resistance of treated corn to Fusarium verticillioides, Fusarium moniliforme, Colletotrichum graminicola, Stenocarpella maydis, and/or Cercospora zeae-maydis; resistance of treated wheat to head blight (Fusarium graminearum), powdery mildew (Erysiphe graminis f. sp. tritici), stripe rust, stem rust or leaf rust (Puccinia tritici); resistance of treated cotton to Fusarium oxysporum and Verticillium dahlia; resistance of treated rice to Magnaporthe oryzae and Rhizoctonia solani, and resistance of treated soybean to Asian rust (Phakopsora pachyrhizi), Phytophthora Root Rot (Phytophthora sp.), White Mold (Sclerotinia sp.), Sudden Death Syndrome (Fusarium virguliforme) and/or Brown Stem Rot (Phialophora gregata).

Inhibitory amounts of a desirable DEF_PFV1 against a pathogenic microbe in a given plant can also be identified by measuring decreases in the adverse effects cause by microbial growth in such plants. Such decreases can be ascertained by comparing the extent of the adverse effect in a DEF_PFV1 treated plant relative to an otherwise identical, control plant treated with a control composition lacking the DEF_PFV1 or comprising the corresponding DEF_PFV2. Adverse effects of microbial growth in a plant that can be measured include any type of plant tissue damage or necrosis, any type of plant yield reduction, any reduction in the value of the crop plant product, and/or production of undesirable microbial metabolites or microbial growth by-products including, but not limited to, mycotoxins. Mycotoxins comprise a number of toxic molecules produced by microbial species, including but not limited to polyketides (including aflatoxins, demethylsterigmatocystin, O-methylsterigmatocystin, etc.), fumonisins, alperisins (e.g., A1s A2, Bls B2), sphingofungins (A, B, C and D), trichothecenes, fumifungins, and the like. Methods of quantitating mycotoxin levels are widely documented. Moreover, commercial kits for measurement of the mycotoxins such as aflatoxin, fumonisin, deoxynivalenol, and zearalenone are also available (VICAM, Watertown, MA, USA).

Infection of certain plants with certain plant pathogen microbes can result in distinctive effects on plant growth that are readily observed. Consequently, one can distinguish plants treated with desirable DEF_PFV1 from a plant treated with a control lacking a Defensin peptide or comprising the corresponding DEF_PFV2 by simply challenging such plants with pathogenic plant microbes and observing reduction of the symptoms normally associated with such infections. Such observations are facilitated by co-infecting otherwise identical, control plants treated with a control lacking a Defensin peptide or comprising the corresponding undesirable DEF_PFV2. Identification of treated plants that control or combat microbial infection can be based on observation of decreased disease symptoms, measurement of the decreased microbial growth in the infected plant (e.g., by determining the numbers of colony forming units per gram of infected tissue) and/or by measurement of the amount of mycotoxin present in infected plant tissue.

Plants of interest include both food crop plants and biofuels or energy crop plants. The plants, parts thereof, and harvested parts thereof (e.g., grain, fruits, and vegetables) to which the methods and compositions disclosed herein can be applied can refer to plants, parts thereof, and harvested parts thereof that are either directly edible, or which produce edible products (e.g., customarily used to feed humans either directly, or indirectly through animals). Plants, parts thereof, and harvested parts thereof used herein include cereal crop (e.g., wheat, rice, maize (corn), barley, oats, sorghum, rye, and millet), legumes (e.g., peanuts, chickpeas, lentils, kidney beans, soybeans, lima beans); root or tuber crop (e.g., potatoes, sweet potatoes, and cassavas); oil crops (e.g., canola (rapeseed), wheat, peanuts, palm, coconuts, safflower, cottonseed, sunflower, flax, and olive); sugar crops (e.g., sugar cane and sugar beets); fruit crops (e.g., bananas, oranges, apples, pears, breadfruit, pineapples, strawberries, grapes, and cherries); vegetable crops and tubers (e.g., tomatoes, lettuce, carrots, melons, asparagus, Brassica sp.); nut crops (e.g., cashews, peanuts, walnuts, pistachio nuts, almonds); forage and turf grasses; forage legumes (e.g., alfalfa, clover); drug crops (e.g., Cannabis sp., coffee, cocoa, kola nut, poppy); spice and flavoring crops (e.g., vanilla, sage, thyme, anise, saffron, menthol, peppermint, spearmint, coriander), fiber crop (e.g., cotton or hemp), and biofuel crops (e.g., castor bean, Camelina, switchgrass, Miscanthus, and Jatropha) plants, parts thereof, and harvested parts thereof.

The analysis of different DEF_PFV to assign particular disulfide bonds can be performed by mass spectrometry-based methods (Weinfurtner, in Oxidative Folding of Proteins: Basic Principles, Cellular Regulation Engineering, 2018, pp. 81-98 DOI: 10.1039/9781788013253-00081; Tang and Speicher, Current protocols in protein science vol. 96, 1 (2019): e86. doi: 10.1002/cpps.86).

Also provided are antimicrobial compositions for agricultural, pharmaceutical, or veterinary use comprising either an antimicrobial plant, or antimicrobial human or veterinary, pathogenic microbe inhibitory amount (“antimicrobial effective amount”) of one or more the present isolated, purified antimicrobial DEF_PFV peptides, or biologically functional equivalents thereof. Such compositions can comprise one, or any combination of, Defensin peptides disclosed herein, and an agriculturally, pharmaceutically, or veterinary-practicably acceptable carrier, diluent, or excipient. As indicated below, other components relevant in agricultural and therapeutic contexts can be included in such compositions as well. The antimicrobial compositions can be used for inhibiting the growth of, or killing, Defensin protein- or peptide-susceptible pathogenic microbes associated with plant, human or animal microbial infections. Such antimicrobial compositions can be formulated for topical administration, and applied topically to either plants, the plant environment (including soil), or humans or animals.

Agricultural compositions comprising any of the present Defensin molecules alone, or in any combination, can be formulated as described in, for example, Winnacker-Kuchler (1986) Chemical Technology, Fourth Edition, Volume 7, Hanser Verlag, Munich; van Falkenberg (1972-1973) Pesticide Formulations, Second Edition, Marcel Dekker, N.Y.; and K. Martens (1979) Spray Drying Handbook, Third Edition, G. Goodwin, Ltd., London. Formulation aids, such as carriers, inert materials, surfactants, solvents, and other additives are also well known in the art, and are described, for example, in Watkins, Handbook of Insecticide Dust Diluents and Carriers, Second Edition, Darland Books, Caldwell, N.J., and Winnacker-Kuchler (1986) Chemical Technology, Fourth Edition, Volume 7, Hanser Verlag, Munich. Using these formulations, it is also possible to prepare compositions where a desired DEF_PFV is combined with other pesticidally active substances, fertilizers, and/or growth regulators, etc., in a composition (e.g., a finished formulation or tank mixes.

Whether alone or in combination with other active agents, the present antimicrobial Defensin peptides can be applied at a concentration in the range of from about 0.1 μg/ml to about 100 mg/ml, or from about 5 μg/ml to about 5 mg/ml, at a pH in the range of from about 3.0 to about 9.0. Such compositions can be buffered using, for example, phosphate buffers between about 1 mM and 1 M, about 10 mM to about 100 mM, or about 15 mM to about 50 mM. In the case of low buffer concentrations, a salt can be added to increase the ionic strength. In certain embodiments, NaCl in the range of from about 1 mM to about 1 M, or about 10 mM to about 100 mM, can be added.

Numerous conventional microbial antibiotics and chemical fungicides with which the present Defensin peptide can be combined are described in Worthington and Walker (1983) The Pesticide Manual, Seventh Edition, British Crop Protection Council. These include, for example, polyoxines, nikkomycines, carboxy amides, aromatic carbohydrates, carboxines, morpholines, inhibitors of sterol biosynthesis, and organophosphorous compounds. In addition, azole, triazole, and/or echinocandin fungicides can also be used. Other active ingredients which can be formulated in combination with the present antimicrobial peptides and proteins include, for example, insecticides, attractants, sterilizing agents, acaricides, nematicides, and herbicides. U.S. Pat. No. 5,421,839, which is incorporated herein by reference in its entirety, contains a comprehensive summary of the many active agents with which substances such as the present antimicrobial Defensin peptides and proteins can be formulated.

The compositions provided herein can be applied in an antimicrobial effective amount, which will vary depending on such factors as, for example, the specific fungal pathogen to be controlled, the specific plant (and plant part or soil) to be treated, and the method of applying the compositions that comprise a desired Defensin peptide.

Desired Defensin peptides and biologically functional equivalents and compositions comprising the same provided herein (e.g., in the following numbered embodiments), can be used to inhibit the growth of a wide variety of susceptible microbes in plants. In certain embodiments, growth of microbes in the following genera or species can be inhibited: Alternaria (e.g., Alternaria brassicicola; Alternaria solani); Ascochyta (e.g., Ascochyta pisi); Aspergillus (e.g., Aspergillus flavus; Aspergillus fumigatus); Botrytis (e.g., Botrytis cinerea); Cercospora (e.g., Cercospora kikuchii; Cercospora zeae-maydis); Colletotrichum (e.g., Colletotrichum lindemuthianum); Diplodia (e.g., Diplodia candis); Erysiphe (e.g., Erysiphe graminis f.sp. graminis; Erysiphe graminis f.sp. hordei); Fusarium (e.g., Fusarium nivale; Fusarium oxysporum; Fusarium graminearum; Fusarium culmorum; Fusarium solani; Fusarium moniliforme; Fusarium roseum); Gaeumanomyces (e.g., Gaeumanomyces graminis f.sp. tritici); Helminthosporium (e.g., Helminthosporium turcicum; Helminthosporium carbonum; Helminthosporium candis); Macrophomina (e.g., Macrophomina phaseolina; Magnaporthe grisea); Nectria (e.g., Nectria heamatococca); Peronospora (e.g., Peronospora manshurica; Peronospora tabacina); Phakopsora (e.g., Phakopsora pachyrhizi); Phoma (e.g., Phoma betae); Phymatotrichum (e.g., Phymatotrichum omnivorum); Phytophthora (e.g., Phytophthora cinnamomi; Phytophthora cactorum; Phytophthora phaseoli; Phytophthora parasitica; Phytophthora citrophthora; Phytophthora sojae; Phytophthora infestans); Plasmopara (e.g., Plasmopara viticola); Podosphaera (e.g., Podosphaera leucotricha); Puccinia (e.g., Puccinia sorghi; Puccinia striiformis; Puccinia graminis f.sp. tritici; Puccinia asparagi; Puccinia recondita; Puccinia arachidis); Pythium (e.g., Pythium aphanidermatum; Pythium ultimum); Pyrenophora (e.g., Pyrenophora tritici-repentens); Pyricularia (e.g., Pyricularia oryzae); Rhizoctonia (e.g., Rhizoctonia solani; Rhizoctonia cerealis); Sclerotium (e.g., Sclerotium rolfsii); Sclerotinia (e.g., Sclerotinia sclerotiorum); Septoria (e.g., Septoria lycopersici; Septoria glycines; Septoria nodorum; Septoria tritici); Thielaviopsis (e.g., Thielaviopsis basicola); Uncinula (e.g., Uncinula necator); Venturia (e.g., Venturia inaequalis); and Verticillium (e.g., Verticillium dahliae; Verticillium albo-atrum).

Pharmaceutical or veterinary compositions that comprise an antimicrobial effective amount of a desired Defensin peptide and a pharmaceutically acceptable or veterinary practicable carrier are also provided. Such pharmaceutical or veterinary compositions can be used for inhibiting the growth of, or killing, susceptible pathogenic microbes that infect humans or animals, i.e., treating such fungal infections by administering to a patient or other subject in need thereof. In certain embodiments, compositions comprising a Defensin peptide, can be formulated by methods adapted from those described in Remington: The Science and Practice of Pharmacy (2005), 21st Edition, University of the Sciences in Philadelphia, Lippincott Williams & Wilkins. In certain embodiments, the compositions can contain a Defensin peptide at concentrations in the range of from about 0.1 μg per ml to about 100 mg per ml, or about 5 μg per ml to about 5 mg per ml, at a pH in the range of from about 3.0 to about 9.0. Such compositions can be buffered using, for example, phosphate buffers at a concentration of about 1 mM to about 1 M, about 10 mM to about 100 mM, or about 15 mM to 50 mM. In the case of low buffer concentrations, a salt can be added to increase the ionic strength. In certain embodiments, NaCl in the range of about 1 mM to about 1 M, or about 10 mM to about 100 mM, can be added.

The Defensin peptide can be formulated alone or can formulated in combination with other conventional antimicrobial therapeutic compounds such as, by way of non-limiting example, polyene antimicrobials; imidazole, triazole, and thiazole antimicrobials; allylamines; and echinocandins that are routinely used in human and veterinary medicine.

Administration of the compositions that comprise a Defensin peptide to a human or animal subject in need thereof can be accomplished via a variety of routes that include topical application, enteral, parenteral, and/or intravenous introduction.

In further embodiments, the present disclosure provides methods for preventing or reducing crop damage or post-harvest loss by a plant pathogenic microbe, the method comprising contacting a plant, a plant seed, pre- or post-harvest grain, pre- or post-harvest fruit, or pre- or post-harvest vegetable with an effective amount of a composition comprising a cationic antimicrobial Defensin peptide folding variant 1 (DEF_PFV1) under conditions that are suitable for preventing or reducing crop damage or post-harvest loss.

In other embodiments, the present disclosure provides medical devices in operable combination with a composition comprising a cationic antimicrobial Defensin peptide folding variant 1 (DEF_PFV1), wherein the medical device comprises at least one surface that is topically coated or impregnated with the composition.

In still further embodiments, provided herein are methods for treating, preventing, or inhibiting a microbial infection in a subject (including a plant, human, or non-human animal) in need thereof, which methods comprise administering (ex vivo or in vivo) to the subject a therapeutically effective amount of a composition comprising a cationic antimicrobial Defensin peptide folding variant 1 (DEF_PFV1) under conditions and for a duration suitable for treating, preventing, or inhibiting the microbial infection.

In related embodiments, provided herein are compositions comprising a cationic antimicrobial Defensin peptide folding variant 1 (DEF_PFV1) for use in a method for treating, preventing, or inhibiting microbial infection in a subject in need thereof.

In other related embodiments, provided herein are plant parts that are at least partly coated with a composition comprising a cationic antimicrobial Defensin peptide folding variant 1 (DEF_PFV1).

### Embodiments

The following numbered embodiments form part of the present disclosure.

1a. A composition comprising a Defensin peptide folding variant 1 (DEF_PFV1), wherein said Defensin peptide is a cationic Defensin peptide comprising in contiguous amino acid sequence from its N-terminal end (1) a first cysteine pair motif including C₁ and C₂, (2) a first intervening sequence (IS1), (3) a second cysteine pair motif including C₃ and C₄, (4) a second intervening sequence (IS2), (5) a third cysteine pair motif including C₅ and C₆, (6) a third intervening sequence (IS3), and (7) a fourth cysteine pair motif including C₇ and C₈, wherein cysteine residues C₁, C₂, C₃, C₄, C₅, C₆, C₇, and C₈ of said DEF_PFV1 form a first set of disulfide bonds and DEF_PFV1 thereby adopts a first conformation that exhibits greater antimicrobial activity, increased protease resistance, or increased protease sensitivity as compared to a Defensin peptide folding variant 2 (DEF_PFV2) that has the same amino acid sequence as DEF_PFV1 but forms a distinct second set of disulfide bonds between cysteine residues C₁, C₂, C₃, C₄, C₅, C₆, C₇, and C₈ and adopts a distinct second conformation, and wherein said composition is substantially free of DEF_PFV2, optionally wherein at least about 80%, 90%, 95%, 98%, 99%, or 99.5% of the total defensin peptide in the composition by weight is DEF_PFV1.

1b. A composition comprising a Defensin peptide folding variant 1 (DEF_PFV1), wherein said Defensin peptide is a C-terminal fragment of a cationic Defensin peptide comprising in contiguous amino acid sequence from its N-terminal end (1) a third cysteine pair motif comprising the conserved C₅ and C₆ of the full length defensin peptide, (2) a third intervening sequence (IS3) of the full length defensin peptide, and (3) a fourth cysteine pair motif including C₇ and C₈ of a full length defensin protein, wherein cysteine residues C₅, C₆, C₇, and C₈ of said DEF_PFV1 form a first set of disulfide bonds and DEF_PFV1 thereby adopts a first conformation that exhibits greater antimicrobial activity, increased protease resistance, or increased protease sensitivity as compared to a Defensin peptide folding variant 2 (DEF_PFV2) that has the same amino acid sequence as DEF_PFV1 but forms a distinct second set of disulfide bonds between cysteine residues C₅, C₆, C₇, and C₈ and adopts a distinct second conformation, and wherein said composition is substantially free of DEF_PFV2, optionally wherein at least about 80%, 90%, 95%, 98%, 99%, or 99.5% of the total defensin peptide in the composition by weight is DEF_PFV1.

2. The composition of embodiment 1a or 1b, wherein said Defensin peptide is a native Defensin peptide.

3. The composition of embodiment 1a or 1b, wherein said Defensin peptide is a synthetic variant of a native Defensin peptide comprising one or more amino acid insertions, deletions, and/or substitutions in a native Defensin peptide.

4. The composition of embodiment 3, wherein said substitutions in a native Defensin peptide comprise at least one of a substitution of a hydrophobic amino acid with a hydrophobic amino acid, a cationic amino acid with a cationic amino acid, or an anionic amino acid with a cationic or anionic amino acid.

5. The composition of any one of embodiments 1 a or 1b to 4, wherein said first set of disulfide bonds comprise a C₁-C₈ disulfide bond, a C₂-C₅ disulfide bond, a C₃-C₆ disulfide bond, and a C₄-C₇ disulfide bond.

6. The composition of embodiment 5, wherein said second set of disulfide bonds comprise disulfide bonds other than a C₁-C₈ disulfide bond, a C₂-C₅ disulfide bond, a C₃-C₆ disulfide bond, and a C₄-C₇ disulfide bond.

7. The composition of any one of embodiments 1 a or 1b to 6, wherein said first cysteine pair motif is a C₁-10AA-C₂ motif that comprises in contiguous sequence from its N-terminal end ten amino acids (A₁, A₂, A₃, A₄, A₅, A₆, A₇, As, A₉, and A₁₀) flanked by an N-terminal C₁ and a C-terminal C₂.

8. The composition of any one of embodiments 1a or 1b to 7, wherein said second cysteine pair motif is a C₃-3AA-C₄ motif that comprises in contiguous sequence from its N-terminal end three amino acids (A₁₁, A₁₂, and A₁₃) flanked by an N-terminal C₃ and a C-terminal C₄.

9. The composition of any one of embodiments 1a or 1b to 8, wherein said third cysteine pair motif is a C₅-5-6AA-C₆ motif that comprises in contiguous sequence from its N-terminal end three amino acids (A₁₄, A₁₅, A₁₆, A₁₇, A₁₈ and, optionally, A₁₉) flanked by an N-terminal C₅ and a C-terminal C₆.

10. The composition of any one of embodiments 1a or 1b to 9, wherein said fourth cysteine pair motif is a C₇-3AA-C₈ motif that comprises in contiguous sequence from its N-terminal end three amino acids (A₂₀, A₂₁, and A₂₂) flanked by an N-terminal C₇ and a C-terminal C₈.

11. A composition comprising a Defensin peptide folding variant 1 (DEF_PFV1), wherein said Defensin peptide is a synthetic variant of a native Defensin peptide comprising in contiguous amino acid sequence from its N-terminal end (1) a first amino acid pair motif including X₁ and C₂, (2) a first intervening sequence (IS1), (3) a first cysteine pair motif including C₃ and C₄, (4) a second intervening sequence (IS2), (5) a second cysteine pair motif including C₅ and C₆, (6) a third intervening sequence (IS3), and (7) a second amino acid pair motif including C₇ and Y₁, wherein said amino acid X₁ and Y₁ are each independently selected from the group of amino acids other than cysteine, and wherein cysteines C₂, C₃, C₄, C₅, C₆, and C₇ of DEF_PFV1 form a first set of disulfide bonds and DEF_PFV1 thereby adopts a first conformation that exhibits greater antimicrobial activity, increased protease resistance, or increased protease sensitivity as compared to a Defensin peptide folding variant 2 (DEF_PFV2) that has the same amino acid sequence as DEF_PFV1 but forms a distinct second set of disulfide bonds between cysteines C₂, C₃, C₄, C₅, C₆, and C₇ and adopts a distinct second conformation, and wherein said composition is substantially free of DEF_PFV2, optionally wherein at least about 80%, 90%, 95%, 98%, 99%, or 99.5% of the total defensin peptide in the composition by weight is DEF_PFV1.

12. The composition of embodiment 11, wherein said amino acids X₁ and Y₁ are each Ser(S) or Thr (T).

13. The composition of embodiment 11 to 12, wherein said first set of disulfide bonds comprise a C₂-C₅ disulfide bond, a C₃-C₆ disulfide bond, and a C₄-C₇ disulfide bond.

14. The composition of any one of embodiments 11 to 13, wherein said second set of disulfide bonds comprise disulfide bonds other than a C₂-C₅ disulfide bond, a C₃-C₆ disulfide bond, and a C₄-C₇ disulfide bond.

15. The composition of embodiment 11 to 14, wherein said first amino acid pair motif is an X₁-10AA-C₂ motif that comprises in contiguous sequence from its N-terminal end ten amino acids (A₁, A₂, A₃, A₄, A₅, A₆, A₇, As, A₉, and A₁₀) flanked by an N-terminal X₁ and a C-terminal C₂.

16. The composition of embodiment 11 to 15, wherein said first cysteine pair motif is a C₃-3AA-C₄ motif that comprises in contiguous sequence from its N-terminal end three amino acids (A₁₁, A₁₂, and A₁₃) flanked by an N-terminal C₃ and a C-terminal C₄.

17. The composition of embodiment 11 to 16, wherein said second cysteine pair motif is a C₅-5-6AA-C₆ motif that comprises in contiguous sequence from its N-terminal end three amino acids (A₁₄, A₁₅, A₁₆, A₁₇, A₁₈ and, optionally, A₁₉) flanked by an N-terminal C₅ and a C-terminal C₆.

18. The composition of embodiment 11 to 17, wherein said second amino acid pair motif is a C₇-3AA-Y₁ motif that comprises in contiguous sequence from its N-terminal end three amino acids (A₂₀, A₂₁, and A₂₂) flanked by an N-terminal C₇ and a C-terminal Y₁.

19. A composition comprising a Defensin peptide folding variant 1 (DEF_PFV1), wherein said Defensin peptide is a synthetic variant of a native Defensin peptide comprising in contiguous amino acid sequence from its N-terminal end (1) a first amino acid pair motif including X₁ and Y₁, (2) a first intervening sequence (IS1), (3) a first cysteine pair motif including C₃ and C₄, (4) a second intervening sequence (IS2), (5) a second cysteine pair motif including C₅ and C₆, (6) a third intervening sequence (IS3), and (7) a second amino acid pair motif including X₂ and Y₂, wherein said amino acids X₁, Y₁, X₂, and Y₂ are each independently selected from the group of amino acids other than cysteine, and wherein cysteines C₃, C₄, C₅, and C₆ of DEF_PFV1 form a first set of disulfide bonds and DEF_PFV1 thereby adopts a first conformation that exhibits greater antimicrobial activity as compared to a Defensin peptide folding variant 2 (DEF_PFV2) that has the same amino acid sequence as DEF_PFV1 but forms a distinct second set of disulfide bonds between cysteines C₃, C₄, C₅, and C₆ and adopts a distinct second conformation, and wherein said composition is substantially free of DEF_PFV2, optionally wherein at least about 80%, 90%, 95%, 98%, 99%, or 99.5% of the total defensin peptide in the composition by weight is DEF_PFV1.

20. The composition of embodiment 19, wherein said amino acids X₁, Y₁, X₂, Y₂ are each Ser(S) or Thr (T).

21. The composition of embodiment 19 to 20, wherein said first amino acid pair motif is a X₁-10AA-Y₁ motif that comprises in contiguous sequence from its N-terminal end ten amino acids (A₁, A₂, A₃, A₄, A₅, A₆, A₇, As, A₉, and A₁₀) flanked by an N-terminal X₁ and a C-terminal Y₁.

22. The composition of embodiment 19 to 21, wherein said first cysteine pair motif is a C₃-3AA-C₄ motif that comprises in contiguous sequence from its N-terminal end three amino acids (A₁₁, A₁₂, and A₁₃) flanked by an N-terminal C₃ and a C-terminal C₄.

23. The composition of embodiment 19 to 22, wherein said second cysteine pair motif is a C₅-5-6AA-C₆ motif that comprises in contiguous sequence from its N-terminal end three amino acids (A₁₄, A₁₅, A₁₆, A₁₇, A₁₈ and, optionally, A₁₉) flanked by an N-terminal C₅ and a C-terminal C₆.

24. The composition of embodiment 19 to 23, wherein said second amino acid pair motif is a X₂-3AA-Y₂ motif that comprises in contiguous sequence from its N-terminal end three amino acids (A₂₀, A₂₁, and A₂₂) flanked by an N-terminal X₂ and a C-terminal Y₁.

25. The composition of any one of embodiments 19 to 24, wherein said Defensin peptide comprises a Defensin gamma core peptide sequence GXC₅X3-9C₆ (SEQ ID NO: 455) or variant gamma core peptide sequence GXC₅X3-10C₆ (SEQ ID NO: 456).

26. The composition of any one of embodiments 19 to 25, wherein said A₁ is an anionic amino acid selected from the group consisting of Asp (D) and Glu (E).

27. The composition of any one of embodiments 19 to 26, wherein said A₄ is a polar neutral amino acid selected from the group consisting of Ser(S) and Thr (T).

28. The composition of any one of embodiments 19 to 27, wherein said A₇ is an aromatic amino acid selected from the group consisting of Phe (F), Tyr (Y), and Trp (W).

29. The composition of any one of embodiments 19 to 28, wherein said A₉ is an aliphatic amino acid selected from the group consisting of Gly (G), Ala (A), Val (V), Leu (L), Ile (I), and Pro (P).

30. The composition of any one of embodiments 19 to 29, wherein said second cysteine pair motif is a C₃-3AA-C₄ motif that comprises in contiguous sequence from its N-terminal end three amino acids (A₁₁, A₁₂, and A₁₃) flanked by an N-terminal C₃ and a C-terminal C₄.

31. The composition of any one of embodiments 19 to 30, wherein said A₁₁ is an aliphatic amino acid selected from the group consisting of Gly (G), Ala (A), Val (V), Leu (L), Ile (I), and Pro (P) or a cationic amino acid selected from the group consisting of His (H), Arg (R), and Lys (K).

32. The composition of any one of embodiments 19 to 31, wherein said A₁₃ is an aliphatic amino acid selected from the group consisting of Gly (G), Ala (A), Val (V), Leu (L), Ile (I), and Pro (P) or a polar neutral amino acid selected from the group consisting of Ser(S) and Thr (T).

33. The composition of any one of embodiments 19 to 32, wherein said third cysteine pair motif is a C₅-5-6AA-C₆ motif that comprises in contiguous sequence from its N-terminal end three amino acids (A₁₄, A₁₅, A₁₆, A₁₇, A₁₈ and, optionally, A₁₉) flanked by an N-terminal C₅ and a C-terminal C₆.

34. The composition of any one of embodiments 19 to 33, wherein said A₁₄ is a cationic amino acid selected from the group consisting of His (H), Arg (R), and Lys (K) or an aliphatic amino acid selected from the group consisting of Gly (G), Ala (A), Val (V), Leu (L), Ile (I), and Pro (P) or a polar neutral amino acid selected from the group consisting of Ser(S) and Thr (T).

35. The composition of any one of embodiments 19 to 34, wherein said A₁₆ is an aliphatic amino acid selected from the group consisting of Gly (G), Ala (A), Val (V), Leu (L), Ile (I), and Pro (P) or an aromatic amino acid selected from the group consisting of Phe (F), Tyr (Y), and Trp (W).

36. The composition of any one of embodiments 19 to 35, wherein said A₁₈ is a cationic amino acid selected from the group consisting of His (H), Arg (R), and Lys (K).

37. The composition of any one of embodiments 19 to 36, wherein said A₂₀ is a polar neutral amino acid selected from the group consisting of Ser(S) and Thr (T).

38. The composition of any one of embodiments 19 to 37, wherein said A₂₁ is a cationic amino acid selected from the group consisting of His (H), Arg (R), and Lys (K).

39. The composition of any one of embodiments 19 to 38, wherein said A₂₂ is an aliphatic amino acid selected from the group consisting of Gly (G), Ala (A), Val (V), Leu (L), Ile (I), and Pro (P).

40. The composition of any one of embodiments 19 to 39, wherein said IS1 comprises five amino acids.

41. The composition of any one of embodiments 19 to 40, wherein said IS1 consists of five amino acids.

42. The composition of any one of embodiments 19 to 41, wherein said IS2 comprises from nine to eleven amino acids.

43. The composition of any one of embodiments 19 to 42, wherein said IS2 consists of nine amino acids.

44. The composition of any one of embodiments 19 to 43, wherein said IS3 comprises one amino acid that is an aliphatic amino acid selected from the group consisting of Gly (G), Ala (A), Val (V), Leu (L), Ile (I), and Pro (P) or an aromatic amino acid selected from the group consisting of Phe (F), Tyr (Y), and Trp (W).

45. The composition of any one of embodiments 19 to 44, wherein said IS3 consists of one amino acid.

46a. A composition comprising a Defensin peptide folding variant 1 (DEF_PFV1) wherein said Defensin is a cationic Defensin peptide comprising a Defensin gamma core peptide sequence GXC₅X3-9C₆ (SEQ ID NO: 455) or a variant gamma core peptide sequence GXC₅X₃-10C₆ (SEQ ID NO: 456), GXC₅X3-9 (F/W/Y) (SEQ ID NO: 457), or GXC₅X3-10 (F/W/Y) (SEQ ID NO: 458) and at least two or three additional cysteine residues selected from the group consisting of cysteine residues corresponding to C₁, C₂, C₃, C₄, C₇, and C₈ of a reference Defensin peptide, wherein said cysteine residues of DEF_PFV1 form a first set of disulfide bonds and DEF_PFV1 thereby adopts a first conformation that exhibits greater antimicrobial activity, increased protease resistance, or increased protease sensitivity as compared to a Defensin peptide folding variant 2 (DEF_PFV2) that has the same amino acid sequence as DEF_PFV1 but forms a distinct second set of disulfide bonds between cysteine residues and adopts a distinct second conformation, and wherein said composition is substantially free of DEF_PFV2, optionally wherein at least about 80%, 90%, 95%, 98%, 99%, or 99.5% of the total defensin peptide in the composition by weight is DEF_PFV1.

46b. A composition comprising a Defensin peptide folding variant 1 (DEF_PFV1) wherein said Defensin is a carboxy terminal (C-terminal) fragment if a cationic Defensin peptide comprising a Defensin gamma core peptide sequence GXC₅X3-9C₆ (SEQ ID NO: 455) or variant gamma core peptide sequence GXC₅X3-10C₆ (SEQ ID NO: 456) and cysteine residues C₇, and C₈ of a reference Defensin peptide, wherein said cysteine residues of DEF_PFV1 form a first set of disulfide bonds and DEF_PFV1 thereby adopts a first conformation that exhibits greater antimicrobial activity, increased protease resistance, or increased protease sensitivity as compared to a Defensin peptide folding variant 2 (DEF_PFV2) that has the same amino acid sequence as DEF_PFV1 but forms a distinct second set of disulfide bonds between cysteine residues and adopts a distinct second conformation, and wherein said composition is substantially free of DEF_PFV2, optionally wherein at least about 80%, 90%, 95%, 98%, 99%, or 99.5% of the total defensin peptide in the composition by weight is DEF_PFV1.

47. The composition of embodiment 46a or b, wherein said cationic Defensin peptide comprises at least four additional cysteine residues selected from the group consisting of cysteines corresponding to C₁, C₂, C₃, C₄, C₅, C₆, C₇, and C₈ of a reference Defensin peptide.

48. The composition of embodiment 46a or b to 47, wherein one to four of the cysteine residues corresponding to C₁, C₂, C₃, C₄, C₇, and C₈ of a reference Defensin peptide are substituted with a different amino acid or are deleted, optionally wherein the cysteine is substituted with a Ser(S) or Thr (T) residue.

49. The composition of embodiment 46a or b to 48, wherein at least one of the cysteine residues corresponding to C₁, C₂, C₃, C₄, C₇, and C₈ of the reference Defensin peptide is substituted with a different amino acid, at least one of the of the cysteine residues corresponding to C₁, C₂, C₃, C₄, C₅, C₆, C₇, and C₈ of the reference Defensin peptide is deleted, and wherein at least two of the cysteine residues corresponding to C₁, C₂, C₃, C₄, C₇, and C₈ of the reference Defensin peptide are retained.

50. The composition of embodiment 46a or b to 49, wherein the Defensin peptide comprises cysteine residues corresponding to C₅, C₆, C₇, and C₈ of a reference Defensin peptide.

51. The composition of embodiment 46a, wherein said Defensin peptide is a native Defensin peptide.

52. The composition of embodiment 46a or b to 51, wherein said Defensin peptide is a synthetic variant of a native Defensin peptide comprising one or more amino acid insertions, deletions, and/or substitutions in a native Defensin peptide.

53. The composition of embodiment 46a or b to 52, wherein said substitutions in a native Defensin peptide comprise at least one of a substitution of a hydrophobic amino acid with a hydrophobic amino acid, a cationic amino acid with a cationic amino acid, or an anionic amino acid with a cationic or anionic amino acid.

54. The composition of embodiment 46a or b to 53, wherein said Defensin peptide comprises a C₅-5-6AA-C₆ motif that comprises in contiguous sequence from its N-terminal end three amino acids (A₁₄, A₁₅, A₁₆, A₁₇, A₁₈ and, optionally, A₁₉) flanked by an N-terminal C₅ and a C-terminal C₆.

55. The composition of embodiment 46a or b to 54, wherein: (i) said A₁₄ is a cationic amino acid selected from the group consisting of His (H), Arg (R), and Lys (K) or an aliphatic amino acid selected from the group consisting of Gly (G), Ala (A), Val (V), Leu (L), Ile (I), and Pro (P) or a polar neutral amino acid selected from the group consisting of Ser(S) and Thr (T); (ii) said A₁₆ is an aliphatic amino acid selected from the group consisting of Gly (G), Ala (A), Val (V), Leu (L), Ile (I), and Pro (P) or an aromatic amino acid selected from the group consisting of Phe (F), Tyr (Y), and Trp (W); and/or (iii) wherein said A₁₈ is a cationic amino acid selected from the group consisting of His (H), Arg (R), and Lys (K).

56. The composition of embodiment 46a or b to 55, wherein said Defensin peptide comprises a C₇-3AA-C₈ motif that comprises in contiguous sequence from its N-terminal end three amino acids (A₂₀, A₂₁, and A₂₂) flanked by an N-terminal C₇ and a C-terminal C₈.

57. The composition of embodiment 46a or b to 56, wherein: (i) said A₂₀ is a polar neutral amino acid selected from the group consisting of Ser(S) and Thr (T); (ii) said A₂₁ is a cationic amino acid selected from the group consisting of His (H), Arg (R), and Lys (K); and/or (iii) said A₂₂ is an aliphatic amino acid selected from the group consisting of Gly (G), Ala (A), Val (V), Leu (L), Ile (I), and Pro (P).

58. The composition of any one of embodiments 46 a or b to 57, wherein said cationic Defensin peptide has an isoelectric point between 8.0 and 12.0.

59. The composition of any one of embodiments 46a or b to 58, wherein said DEF_PFV1 exhibits greater protease resistance than said DEF_PFV2.

60. The composition of any one of embodiments 46a or b to 59, wherein said DEF_PFV1 exhibits lower protease resistance than said DEF_PFV2.

61. The composition of any one of embodiments 46a or b to 60, wherein said Defensin peptide comprises an amino acid sequence having at least 60%, 70%, 75%, 80%, 85%, 90%, 95%, 97%, or 99% sequence identity to any one of SEQ ID NO: 1 to 454 or SEQ ID NO: 459 to SEQ ID NO: 472.

62. The composition of any one of embodiments 46a or b to 61, wherein said composition further comprises an agriculturally, pharmaceutically, or veterinary practicable carrier, diluent, or excipient.

63. A method for making a composition of any one of embodiments 1a or b to 62, said method comprising: (a) separating a fraction comprising Defensin peptide folding variant 1 (DEF_PFV1) from one or more fractions comprising Defensin peptide folding variant 2 (DEF_PFV2), or DEF_PFV2 peptide fragments thereof, from a mixture comprising DEF_PFV1 and DEF_PFV2, or DEF_PFV2 peptide fragments thereof; and (b) combining said fraction comprising DEF_PFV1, or a preparation further purified from said DEF_PFV1 fraction, with an agriculturally, pharmaceutically, or veterinary practicable carrier, diluent, or excipient, thereby making the composition.

64. The method of embodiment 63, further comprising obtaining said mixture comprising DEF_PFV1 and DEF_PFV2, or DEF_PFV2 peptide fragments thereof, by (a) culturing in fermentation broth microorganisms expressing a recombinant polynucleotide comprising a transcriptional promoter operably linked to a polynucleotide encoding a signal peptide that is in frame with and upstream of a polynucleotide encoding DEF_PFV1, or a variant thereof, and (b) separating the microorganisms from the fermentation broth, which comprises the mixture, wherein said microorganisms are optionally selected from the group consisting of Candida, Kluveromyces, Hansenula, Pichia, Saccharomyces, Schizosaccharomyces, or Yarrowia, Myceliophthora, and Escherichia.

65. The method of embodiment 63 to 64, further comprising obtaining said mixture comprising DEF_PFV1 and DEF_PFV2, or DEF_PFV2 peptide fragments thereof, by (a) culturing in fermentation broth microorganisms expressing a recombinant polynucleotide comprising a transcriptional promoter operably linked to a polynucleotide encoding DEF_PFV1, or a variant thereof, and (b) separating the microorganisms, which comprises the mixture, from the fermentation broth, (c) disrupting the microorganisms to obtain a cell lysate, and (d) separating insoluble debris in the cell lysate from an aqueous fraction comprising said mixture, wherein said microorganisms are optionally selected from the group consisting of Candida, Kluveromyces, Hansenula, Pichia, Saccharomyces, Schizosaccharomyces, or Yarrowia, Myceliophthora, and Escherichia.

66. The method of embodiment 63 to 65, further comprising treating said mixture comprising DEF_PFV1 and DEF_PFV2 with a protease under non-denaturing conditions that are sufficient to produce DEF_PFV2 peptide fragments but insufficient to produce DEF_PFV1 peptide fragments.

67. The method of embodiment 63 to 66, wherein the protease is a trypsin family serine protease that cleaves DEF_PFV2 at the carboxy terminus of a DEF_PFV2 Arg (R) or Lys (K).

68. The method of embodiment 63 to 67, wherein the trypsin family serine protease is a recombinant bovine, porcine, human, or microbial trypsin, and, optionally, wherein the microbial trypsin is a Streptomycete trypsin or variant thereof.

69. The method of embodiment 63 to 68, further comprising separating the DEF_PFV1 from the DEF_PFV2 peptide fragments by size-exclusion chromatography.

70. A method for preventing or reducing crop damage or post-harvest loss by a plant pathogenic microbe, said method comprising contacting a plant, a plant seed, pre- or post-harvest grain, pre- or post-harvest fruit, or pre- or post-harvest vegetable with an effective amount of the composition of any one of embodiments 1a or b-62, and under conditions suitable for preventing or reducing crop damage or post-harvest loss.

71. The method of embodiment 70, wherein the plant pathogenic microbe is selected from the group consisting of Fusarium sp., Alternaria sp., Verticillium sp., Phytophthora sp., Colletotrichum sp., Botrytis sp., Cercospora sp., Phakopsora sp., Rhizoctonia sp., Sclerotinia sp., Pythium sp., Phoma sp., Leptosphaeria sp., Gaeumannomyces sp., Puccinia sp., Septoria sp., Penicillium sp., Lasiodiplodia sp., Phomopsis sp., Mycosphaerella sp., Golovinomyces sp., Erisyphe sp., Albugo sp., Setosphaeria sp., Cochliobolus sp., Helminthosporium sp., Diplodia sp. or Stenocarpella sp.

72. The method of embodiment 70 to 71, wherein the plant, plant seed, pre- or post-harvest grain, pre- or post-harvest fruit, or pre- or post-harvest vegetable is selected from the group consisting of a cereal crop, a legume crop, a root or tuber crop, an oil crop, a fruit crop, a vegetable crop, a nut crop, a forage or turf grass crop, a forage legume, a drug crop, a spice or flavoring crop, a fiber crop, and a biofuel crop plant, a plant seed, pre- or post-harvest grain, pre- or post-harvest fruit, or pre- or post-harvest vegetable.

73. The method of embodiment 70 to 72, wherein the plant, a plant seed, pre- or post-harvest grain, pre- or post-harvest fruit, or pre- or post-harvest vegetable is selected from the group consisting of wheat, rice, corn, barley, oat, sorghum, rye, millet, peanut, chickpea, pigeon pea, lentil, kidney bean, soybean, lima bean, potato, sweet potato, cassava, canola, peanut, palm, coconut, safflower, cotton, sunflower, flax, olive, sugarcane, sugarbeet, banana, orange, apple, pear, breadfruit, pineapple, strawberry, grape, cherry, coffee, coco, tomato, lettuce, carrot, melon, asparagus, Brassica sp., cashew, walnut, pistachio, almond, alfalfa, clover, castor, Camelina, pennycress, switchgrass, Miscanthus, and Jatropha plant, plant seed, pre- or post-harvest grain, pre- or post-harvest fruit, or pre- or post-harvest vegetable.

74. A medical device having antimicrobial properties, said medical device comprising a medical device in operable combination with a composition of any one of embodiments 1a or b-62, wherein said medical device comprises at least one surface that is topically coated or impregnated with said composition.

75. The medical device of embodiment 74, wherein said medical device is selected from the group consisting of a stent, a catheter, a contact lens, a condom, a patch, and a diaphragm.

76. A method for treating, preventing, or inhibiting a microbial infection in a subject in need thereof, said method comprising administering to said subject a therapeutically effective amount of a composition of any one of embodiments 1-62 under conditions and for a duration suitable for treating, preventing, or inhibiting said microbial infection.

77. The method of embodiment 76, wherein said administering comprises topical, enteral, parenteral, or intravenous introduction of the composition to the subject.

78. The method of embodiment 76 to 77, wherein the subject is selected from the group consisting of human, livestock, poultry, fish, and companion animal.

79. The method of embodiment 76 to 78, wherein the microbial infection is of a mucosal membrane, eye, skin, or a nail and the composition is applied to the mucosal membrane, eye, skin, or nail.

80. The method of embodiment 76 to 79, wherein the microbial infection is by a dermatophyte, and wherein the dermatophyte is optionally selected from the group consisting of Trichophyton rubrum, Trichophyton interdigitale, Trichophyton violaceum, Trichophyton tonsurans, Trichophyton soudanense, Trichophyton mentagrophytes, Microsporum flavum, Epidermophyton floccosum, and Microsporum gypseum.

81. The method of embodiment 76 to 80, wherein the microbial infection is by a microorganism selected from the group consisting of Aspergillus, Cryptococcus, Penicillium, Rhizopus, Apophysomyces, Cunninghamella, Saksenaea, Rhizomucor, Syncephalostrum, Cokeromyces, Actinomucor, Pythium, Fusarium, Histoplasmosis, and Blastomyces species.

82. The method of embodiment 76 to 81, wherein the microbial infection is by a microorganism selected from the group consisting of Candida species, wherein the Candida species is selected from the group consisting of Candida albicans (C. albicans), C. auris, C. glabrata, C. parasilosis, C. tropicalis, and C. krusei.

83. The composition of any one of embodiments 1a or b-62 for use in a method for treating, preventing, or inhibiting microbial infection in a subject in need thereof.

84. The composition of embodiment 83, wherein the subject is selected from the group consisting of human, livestock, poultry, fish, and companion animals.

85. A plant part that is at least partly coated with the composition of any one of embodiments 1-62.

86. The plant part of embodiment 85, wherein said plant part is selected from the group consisting of a seed and a pre- or post-harvest grain.

87. The plant part of embodiment 85 to 86, wherein said plant part is selected from the group consisting of pre- or post-harvest fruit, pre- or post-harvest vegetable, and pre- or post-harvest flower.

88. The plant part of embodiment 85 to 87, wherein the plant part is selected from the group consisting of cereal crop, legume crop, root or tuber crop, oil crop, fruit crop, vegetable crop, nut crop, forage or turf grass crop, forage legume, drug crop, spice or flavoring crop, fiber crop, and biofuel crop plant part.

89. The plant part of embodiment 85 to 88, wherein the plant part is selected from the group consisting of wheat, rice, corn, barley, oat, sorghum, rye, millet, peanut, chickpea, pigeon pea, lentil, kidney bean, soybean, lima bean, potato, sweet potato, cassava, canola, peanut, palm, coconut, safflower, cotton, sunflower, flax, olive, sugarcane, sugarbeet, banana, orange, apple, pear, breadfruit, pineapple, strawberry, grape, cherry, coffee, coco, tomato, lettuce, carrot, melon, asparagus, Brassica sp., cashew, walnut, pistachio, almond, alfalfa, clover, castor, Camelina, pennycress, switchgrass, Miscanthus, and Jatropha plant part.

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Where a term is provided in the singular, other embodiments described by the plural of that term are also provided. As used herein, the terms “include,” “includes,” and “including” are to be construed as at least having the features to which they refer while not excluding any additional unspecified features. It will be understood that, unless indicated to the contrary, terms intended to be “open” (e.g., the term “including” should be interpreted as “including but not limited to,” the term “having” should be interpreted as “having at least,” the term “includes” should be interpreted as “includes but is not limited to,” etc.). Phrases such as “at least one,” and “one or more,” and terms such as “a” or “an” include both the singular and the plural.

It will be further understood that where features or aspects of the disclosure are described in terms of Markush groups, the disclosure is also intended to be described in terms of any individual member or subgroup of members of the Markush group. Similarly, all ranges disclosed herein also encompass all possible sub-ranges and combinations of sub-ranges and that language such as “between,” “up to,” “at least,” “greater than,” “less than,” and the like include the number recited in the range and includes each individual member.

All references cited herein, whether supra or infra, including, but not limited to, patents, patent applications, and patent publications, whether U.S., PCT, or non-U.S. foreign, and all technical and/or scientific publications are hereby incorporated by reference in their entirety.

### EXAMPLES

While various embodiments have been disclosed herein, other embodiments will be apparent to those skilled in the art. The various embodiments disclosed herein are for purposes of illustration and are not intended to be limiting, with the true scope and spirit being indicated by the claims. The present disclosure is further described with reference to the following examples, which are provided to illustrate certain embodiments and are not intended to limit the scope of the present disclosure or the subject matter claimed.

### Example 1: Heterologous Expression and Purification of DEF_PFV1 and DEF_PFV2

A synthetic defensin gene encoding a Defensin peptide (see, Tables 1 and 2) is cloned into a SacI linearized pPICZa-A integration vector (Invitrogen, Carlsbad, CA) at XhoI and XbaI restriction sites in-frame with the α-mating factor secretion signal sequence containing KEX2 cleavage site without the Glu-Ala repeats and expressed in Pichia pastoris X₃₃.

Defensin peptides are purified using CM-Sephadex C-25 cation-exchange chromatography and C₁₈ reverse-phase HPLC as described, with minor modifications, in Sagaram, PLoS One 6 (4): e18550 (2011). After induction, cells are harvested by centrifugation at 6,000 rpm at 4° C. for 20 minutes, and the pH of the supernatant is adjusted to 6.0. A cation-exchange resin (CM-Sephadex C-25, Sigma, Cat no: C₂₅₁₂₀) is equilibrated with binding buffer (25 mM sodium acetate, pH 6.0), added to the supernatant, and incubated overnight at 4° C. at 110 rpm. After collecting and washing the resin with a binding buffer, bound proteins are eluted with elution buffer (1M NaCl, 50 mM Tris, pH 7.6) using AKTA FPLC. An FPLC fraction containing DEF peptide is concentrated using an Amicon Ultra-15 Centrifugal filter unit. Concentrated fractions are dialyzed against 10 mM Tris, pH 7.6 and further purified by reverse phase C₁₈-HPLC. Peaks are observed and fractions containing DEF_PFV1 (Peak1) and DEF_PFV2 (Peak2) are collected using the method provided in the Agilent HPLC system manual (Agilent Technologies Part No. G1380-90000 (1999)). HPLC fractions containing DEF_PFV1 (Peak1) and DEF_PFV2 (Peak2) peptides are lyophilized and resuspended in nuclease-free water. The concentration of DEF_PFV1 and DEF_PFV2 are determined using a BCA assay performed according to the manufacturer's protocol (Thermo-Fisher Scientific, Inc.® “Protein Assay Technical Handbook”). The purity and size of DEF_PFV1 and DEF_PFV2 are verified by sodium dodecyl sulfate-polyacrylamide gel electrophoresis (SDS-PAGE). DEF_PFV1 and DEF_PFV2 peptides are subjected to NMR analysis to determine the disulfide bonding pairs present in each peptide.

### Example 2: Fungal Cultures and Spore Suspensions

Fungal strains of Botrytis cinerea T-4 are cultured in 20% V8 growth medium. Alternaria alternata is grown on potato dextrose agar. Fungal spores are harvested by flooding fungal growth plates with sterile water. A spore suspension is filtered through two layers of Miracloth, centrifuged at 13,600 rpm for 1 min, washed, and re-suspended in low-salt Synthetic Fungal Medium (SFM) (see, U.S. Pat. No. 6,916,970). Spore suspensions are adjusted to equivalent spore density using a hemocytometer.

Cercospora sojina and Alternaria alternata conidia are harvested from a fully grown culture plate by gently dislodging the conidia with an L-spreader in the presence of 5 ml sterile water. To remove mycelial fragments, a spore suspension is filtered through three layers of miracloth and washed twice with sterile water at 13,000 rpm for 2 minutes. Spore concentration is adjusted to 1×10⁵ spores/ml using a hemocytometer.

Sclerotinia sclerotiorum (S. sclerotiorum) 555 fungal cultures are grown at room temperature on potato dextrose agar (PDA) medium (BD Diagnostics, Sparks, MD “Difco™ & BBL™ Manual, Second Edition”).

### Example 3: Determination of Minimum Inhibitory Concentration (MIC) of DEF_PFV1 and DEF_PFV2 in Absence and Presence of Cations

Antifungal activity of DEF_PFV1 and DEF_PFV2 is assessed at different concentrations using a 2-fold dilution series of each peptide. Antifungal activity of each peptide is determined spectrophotometrically using the 96-well plate assay (Sagaram, PLoS One 6 (4): e18550 (2011) and Sagaram, PLoS One 8 (12): e82485 (2013)). Around 45 μL of each peptide at different concentrations is added to each well of the microtiter plate containing 45 μL of ~10⁵ spores/ml spore suspension. Quantitative fungal growth inhibition is determined by measuring the absorbance at 595 nm using a microplate reader (Tecan Infinite® M200 Pro, Tecan Systems Inc., San Jose, CA) after 48 h. Fungal cell viability is determined by a resazurin cell viability assay (see, Li, MPMI 32:1649-1664 (2019) and Velivelli, PNAS 117 (27): 16043 (2020).

After incubation of a pathogen/peptide mixture for 48 h, 10 μl of 0.1% resazurin solution is added to each well. After incubation of the mixture overnight, a change in the color of the resazurin dye from blue to pink or colorless indicates the presence of live fungal cells. The MIC for each peptide is the lowest concentration of each peptide at which no change in blue color occurs. Using this protocol, the MIC value of DEF_PFV1 and DEF_PFV2 is determined in presence of 100 mM NaCl and 2 mM CaCl₂.

MIC assays of DEF_PFV1 and DEF_PFV2 are carried out against B. cinerea in 2× SFM to determine antifungal activity. It has been hypothesized antifungal peptides significantly lose their antifungal activity in presence of cations since electrostatic interactions between a positively charged peptide and negatively charged fungal membranes are significantly weakened in presence of cations. Antifungal activity of DEF_PFV1 and DEF_PFV2 can, therefore, be determined in SFM supplemented with 100 mM NaCl or 2 mM CaCl₂). Loss of antifungal activity in the presence of either NaCl or CaCl₂) indicates that antifungal activity is salt-sensitive.

### Example 4: Semi-In Planta Antifungal Activity of DEF_PFV Against S. sclerotiorum 555

For semi-in planta antifungal assays, a 1 mm plug is taken from the leading edge of freshly cultured 2-day old fungal colony from PDA media and placed on approximately 3-4 weeks of age detached soybean leaves and pods (6 weeks of age). Then, 40 μL solution of DEF_PFV at various concentrations or water is applied immediately on the plug and allowed to incubate under high humidity for 2-3 days prior to assessment. The severity of disease lesions on each leaf is assessed using the CropReporter system as described in Li et al. Mol Plant Microbe Interact. 2019 December; 32 (12): 1649-1664. doi: 10.1094/MPMI-8-19-0224-R. Epub 2019 Oct. 28. PMID: 31425003. High-resolution fluorescence images can be taken using CropReporter (PhenoVation, Wageningen, Netherlands).

### Example 5: Antifungal Activity of DEF_PFV1 and DEF_PFV2 Against Botrytis cinerea Infection in Tomato Fruits

Antifungal activity assays are performed on commercially available tomato fruits. Tomato fruits are washed with 0.01% bleach and rinsed three times with sterile distilled water. Carborundum Powder (Fisher Chemical, USA) is gently rubbed on the fruit surface to create microinjuries. The fruits are inoculated with 20 μl B. cinerea conidia (1×10⁵ conidia ml-1) via drop inoculation. Conidia of B. cinerea are prepared as described above. Fruits are incubated in a Ziploc weather shield box containing moist paper towels to maintain high humidity after drop inoculation. 20 μl of peptide solution (0.75 μM of DEF_PFV1 or DEF_PFV2) is applied to the drop inoculated conidial suspension at 12 hours post-inoculation and incubated for 5 days. The fruits are observed for fungal growth daily.

What is claimed is:

1. A composition comprising a Defensin peptide folding variant 1 (DEF_PFV1),
wherein said Defensin peptide is a cationic Defensin peptide comprising in contiguous amino acid sequence from its N-terminal end (1) a first cysteine pair motif including C₁ and C₂, (2) a first intervening sequence (IS1), (3) a second cysteine pair motif including C₃ and C₄, (4) a second intervening sequence (IS2), (5) a third cysteine pair motif including C₅ and C₆, (6) a third intervening sequence (IS3), and (7) a fourth cysteine pair motif including C₇ and C₈,
wherein cysteine residues C₁, C₂, C₃, C₄, C₅, C₆, C₇, and C₈ of said DEF_PFV1 form a first set of disulfide bonds and DEF_PFV1 thereby adopts a first conformation that exhibits greater antimicrobial activity, increased protease resistance, or increased protease sensitivity as compared to a Defensin peptide folding variant 2 (DEF_PFV2) that has the same amino acid sequence as DEF_PFV1 but forms a distinct second set of disulfide bonds between cysteine residues C₁, C₂, C₃, C₄, C₅, C₆, C₇, and C₈ and adopts a distinct second conformation, and
wherein said composition is substantially free of DEF_PFV2.

2. The composition of claim 1, wherein said Defensin peptide is a native Defensin peptide.

3. The composition of claim 1, wherein said Defensin peptide is a synthetic variant of a native Defensin peptide comprising one or more amino acid insertions, deletions, and/or substitutions in a native Defensin peptide.

4. The composition of claim 1, wherein said substitutions in a native Defensin peptide comprise at least one of a substitution of a hydrophobic amino acid with a hydrophobic amino acid, a cationic amino acid with a cationic amino acid, or an anionic amino acid with a cationic or anionic amino acid.

5. The composition of claim 1, wherein said first set of disulfide bonds comprise a C₁-C₈ disulfide bond, a C₂-C₅ disulfide bond, a C₃-C₆ disulfide bond, and a C₄-C₇ disulfide bond.

6. The composition of claim 1, wherein said second set of disulfide bonds comprise disulfide bonds other than a C₁-C₈ disulfide bond, a C₂-C₅ disulfide bond, a C₃-C₆ disulfide bond, and a C₄-C₇ disulfide bond.

7. The composition of claim 1, wherein said first cysteine pair motif is a C₁-10AA-C₂ motif that comprises in contiguous sequence from its N-terminal end ten amino acids (A₁, A₂, A₃, A₄, A₅, A₆, A₇, As, A₉, and A₁₀) flanked by an N-terminal C₁ and a C-terminal C₂.

8. The composition of claim 1, wherein said second cysteine pair motif is a C₃-3AA-C₄ motif that comprises in contiguous sequence from its N-terminal end three amino acids (A₁₁, A₁₂, and A₁₃) flanked by an N-terminal C₃ and a C-terminal C₄.

9. The composition of claim 1, wherein said third cysteine pair motif is a C₅-5-6AA-C₆ motif that comprises in contiguous sequence from its N-terminal end three amino acids (A₁₄, A₁₅, A₁₆, A₁₇, A₁₈ and, optionally, A₁₉) flanked by an N-terminal C₅ and a C-terminal C₆.

10. The composition of claim 1, wherein said fourth cysteine pair motif is a C₇-3AA-C₈ motif that comprises in contiguous sequence from its N-terminal end three amino acids (A₂₀, A₂₁, and A₂₂) flanked by an N-terminal C₇ and a C-terminal C₈.

11. A composition comprising a Defensin peptide folding variant 1 (DEF_PFV1),
wherein said Defensin peptide is a synthetic variant of a native Defensin peptide comprising in contiguous amino acid sequence from its N-terminal end (1) a first amino acid pair motif including X₁ and C₂, (2) a first intervening sequence (IS1), (3) a first cysteine pair motif including C₃ and C₄, (4) a second intervening sequence (IS2), (5) a second cysteine pair motif including C₅ and C₆, (6) a third intervening sequence (IS3), and (7) a second amino acid pair motif including C₇ and Y₁,
wherein said amino acid X₁ and Y₁ are each independently selected from the group of amino acids other than cysteine, and
wherein cysteines C₂, C₃, C₄, C₅, C₆, and C₇ of DEF_PFV1 form a first set of disulfide bonds and DEF_PFV1 thereby adopts a first conformation that exhibits greater antimicrobial activity increased protease resistance, or increased protease sensitivity as compared to a Defensin peptide folding variant 2 (DEF_PFV2) that has the same amino acid sequence as DEF_PFV1 but forms a distinct second set of disulfide bonds between cysteines C₂, C₃, C₄, C₅, C₆, and C₇ and adopts a distinct second conformation, and
wherein said composition is substantially free of DEF_PFV2.

12. The composition of claim 11, wherein said amino acids X₁ and Y₁ are each Ser(S) or Thr (T).

13. The composition of claim 11, wherein said first set of disulfide bonds comprise a C₂-C₅ disulfide bond, a C₃-C₆ disulfide bond, and a C₄-C₇ disulfide bond.

14. The composition of claim 11, wherein said second set of disulfide bonds comprise disulfide bonds other than a C₂-C₅ disulfide bond, a C₃-C₆ disulfide bond, and a C₄-C₇ disulfide bond.

15. The composition of claim 11, wherein said first amino acid pair motif is an X₁-10AA-C₂ motif that comprises in contiguous sequence from its N-terminal end ten amino acids (A₁, A₂, A₃, A₄, A₅, A₆, A₇, As, A₉, and A₁₀) flanked by an N-terminal X₁ and a C-terminal C₂.

16. The composition of claim 11, wherein said first cysteine pair motif is a C₃-3AA-C₄ motif that comprises in contiguous sequence from its N-terminal end three amino acids (A₁₁, A₁₂, and A₁₃) flanked by an N-terminal C₃ and a C-terminal C₄.

17. The composition of claim 11, wherein said second cysteine pair motif is a C₅-5-6AA-C₆ motif that comprises in contiguous sequence from its N-terminal end three amino acids (A₁₄, A₁₅, A₁₆, A₁₇, A₁₈ and, optionally, A₁₉) flanked by an N-terminal C₅ and a C-terminal C₆.

18. The composition of claim 11, wherein said second amino acid pair motif is a C₇-3AA-Y₁ motif that comprises in contiguous sequence from its N-terminal end three amino acids (A₂₀, A₂₁, and A₂₂) flanked by an N-terminal C₇ and a C-terminal Y₁.

19. A composition comprising a Defensin peptide folding variant 1 (DEF_PFV1),
wherein said Defensin peptide is a synthetic variant of a native Defensin peptide comprising in contiguous amino acid sequence from its N-terminal end (1) a first amino acid pair motif including X₁ and Y₁, (2) a first intervening sequence (IS1), (3) a first cysteine pair motif including C₃ and C₄, (4) a second intervening sequence (IS2), (5) a second cysteine pair motif including C₅ and C₆, (6) a third intervening sequence (IS3), and (7) a second amino acid pair motif including X₂ and Y₂,
wherein said amino acids X₁, Y₁, X₂, and Y₂ are each independently selected from the group of amino acids other than cysteine, and
wherein cysteines C₃, C₄, C₅, and C₆ of DEF_PFV1 form a first set of disulfide bonds and DEF_PFV1 thereby adopts a first conformation that exhibits greater antimicrobial activity, increased protease resistance, or increased protease sensitivity as compared to a Defensin peptide folding variant 2 (DEF_PFV2) that has the same amino acid sequence as DEF_PFV1 but forms a distinct second set of disulfide bonds between cysteines C₃, C₄, C₅, and C₆ and adopts a distinct second conformation, and
wherein said composition is substantially free of DEF_PFV2.

20. The composition of claim 19, wherein said amino acids X₁, Y₁, X₂, Y₂ are each Ser(S) or Thr (T).

21. The composition of claim 19, wherein said first amino acid pair motif is a X₁-10AA-Y₁ motif that comprises in contiguous sequence from its N-terminal end ten amino acids (A₁, A₂, A₃, A₄, A₅, A₆, A₇, A₈, A₉, and A₁₀) flanked by an N-terminal X₁ and a C-terminal Y₁.

22. The composition of claim 19, wherein said first cysteine pair motif is a C₃-3AA-C₄ motif that comprises in contiguous sequence from its N-terminal end three amino acids (A₁₁, A₁₂, and A₁₃) flanked by an N-terminal C₃ and a C-terminal C₄.

23. The composition of claim 19, wherein said second cysteine pair motif is a C₅-5-6AA-C₆ motif that comprises in contiguous sequence from its N-terminal end three amino acids (A₁₄, A₁₅, A₁₆, A₁₇, A₁₈ and, optionally, A₁₉) flanked by an N-terminal C₅ and a C-terminal C₆.

24. The composition of claim 19, wherein said second amino acid pair motif is a X₂-3AA-Y₂ motif that comprises in contiguous sequence from its N-terminal end three amino acids (A₂₀, A₂₁, and A₂₂) flanked by an N-terminal X₂ and a C-terminal Y₁.

25. The composition of any one of claims 19 to 24, wherein said Defensin peptide comprises a Defensin gamma core peptide sequence GXC₅X3-9C₆ (SEQ ID NO: 455) or variant gamma core peptide sequence GXC₅X3-10C₆ (SEQ ID NO: 456).

26. The composition of any one of claims 19 to 24, wherein said A₁ is an anionic amino acid selected from the group consisting of Asp (D) and Glu (E).

27. The composition of any one of claims 19 to 24, wherein said A₄ is a polar neutral amino acid selected from the group consisting of Ser(S) and Thr (T).

28. The composition of any one of claims 19 to 24, wherein said A₇ is an aromatic amino acid selected from the group consisting of Phe (F), Tyr (Y), and Trp (W).

29. The composition of any one of claims 19 to 24, wherein said A₉ is an aliphatic amino acid selected from the group consisting of Gly (G), Ala (A), Val (V), Leu (L), Ile (I), and Pro (P).

30. The composition of any one of claims 19 to 24, wherein said second cysteine pair motif is a C₃-3AA-C₄ motif that comprises in contiguous sequence from its N-terminal end three amino acids (A₁₁, A₁₂, and A₁₃) flanked by an N-terminal C₃ and a C-terminal C₄.

31. The composition of any one of claims 19 to 24, wherein said A₁₁ is an aliphatic amino acid selected from the group consisting of Gly (G), Ala (A), Val (V), Leu (L), Ile (I), and Pro (P) or a cationic amino acid selected from the group consisting of His (H), Arg (R), and Lys (K).

32. The composition of any one of claims 19 to 24, wherein said A₁₃ is an aliphatic amino acid selected from the group consisting of Gly (G), Ala (A), Val (V), Leu (L), Ile (I), and Pro (P) or a polar neutral amino acid selected from the group consisting of Ser(S) and Thr (T).

33. The composition of any one of claims 19 to 24, wherein said third cysteine pair motif is a C₅-5-6AA-C₆ motif that comprises in contiguous sequence from its N-terminal end three amino acids (A₁₄, A₁₅, A₁₆, A₁₇, A₁₈ and, optionally, A₁₉) flanked by an N-terminal C₅ and a C-terminal C₆.

34. The composition of any one of claims 19 to 24, wherein said A₁₄ is a cationic amino acid selected from the group consisting of His (H), Arg (R), and Lys (K) or an aliphatic amino acid selected from the group consisting of Gly (G), Ala (A), Val (V), Leu (L), Ile (I), and Pro (P) or a polar neutral amino acid selected from the group consisting of Ser(S) and Thr (T).

35. The composition of any one of claims 19 to 24, wherein said A₁₆ is an aliphatic amino acid selected from the group consisting of Gly (G), Ala (A), Val (V), Leu (L), Ile (I), and Pro (P) or an aromatic amino acid selected from the group consisting of Phe (F), Tyr (Y), and Trp (W).

36. The composition of any one of claims 19 to 24, wherein said A₁₈ is a cationic amino acid selected from the group consisting of His (H), Arg (R), and Lys (K).

37. The composition of any one of claims 19 to 24, wherein said A₂₀ is a polar neutral amino acid selected from the group consisting of Ser(S) and Thr (T).

38. The composition of any one of claims 19 to 24, wherein said A₂₁ is a cationic amino acid selected from the group consisting of His (H), Arg (R), and Lys (K).

39. The composition of any one of claims 19 to 24, wherein said A₂₂ is an aliphatic amino acid selected from the group consisting of Gly (G), Ala (A), Val (V), Leu (L), Ile (I), and Pro (P).

40. The composition of any one of claims 19 to 24, wherein said IS1 comprises five amino acids.

41. The composition of any one of claims 19 to 24, wherein said IS1 consists of five amino acids.

42. The composition of any one of claims 19 to 24, wherein said IS2 comprises from nine to eleven amino acids.

43. The composition of any one of claims 19 to 24, wherein said IS2 consists of nine amino acids.

44. The composition of any one of claims 19 to 24, wherein said IS3 comprises one amino acid that is an aliphatic amino acid selected from the group consisting of Gly (G), Ala (A), Val (V), Leu (L), Ile (I), and Pro (P) or an aromatic amino acid selected from the group consisting of Phe (F), Tyr (Y), and Trp (W).

45. The composition of any one of claims 19 to 24, wherein said IS3 consists of one amino acid.

46. A composition comprising a Defensin peptide folding variant 1 (DEF_PFV1),
wherein said Defensin is a cationic Defensin peptide comprising a Defensin gamma core peptide sequence GXC₅X3-9C₆ (SEQ ID NO: 455) or variant gamma core peptide sequence GXC₅X3-10C₆ (SEQ ID NO: 456), GXC₅X3-9 (F/W/Y) (SEQ ID NO: 457), or GXC₅X3-10 (F/W/Y) (SEQ ID NO: 458) and at least two or three additional cysteine residues selected from the group consisting of cysteine residues corresponding to C₁, C₂, C₃, C₄, C₇, and C₈ of a reference Defensin peptide,
wherein said cysteine residues of DEF_PFV1 form a first set of disulfide bonds and DEF_PFV1 thereby adopts a first conformation that exhibits greater antimicrobial activity, increased protease resistance, or increased protease sensitivity as compared to a Defensin peptide folding variant 2 (DEF_PFV2) that has the same amino acid sequence as DEF_PFV1 but forms a distinct second set of disulfide bonds between cysteine residues and adopts a distinct second conformation, and
wherein said composition is substantially free of DEF_PFV2.

47. The composition of claim 46, wherein said cationic Defensin peptide comprises at least four additional cysteine residues selected from the group consisting of cysteines corresponding to C₁, C₂, C₃, C₄, C₅, C₆, C₇, and C₈ of a reference Defensin peptide.

48. The composition of claim 46, wherein one to four of the cysteine residues corresponding to C₁, C₂, C₃, C₄, C₇, and C₈ of a reference Defensin peptide are substituted with a different amino acid or are deleted, optionally wherein the cysteine is substituted with a Ser(S) or Thr (T) residue.

49. The composition of claim 46, wherein at least one of the cysteine residues corresponding to C₁, C₂, C₃, C₄, C₇, and C₈ of the reference Defensin peptide is substituted with a different amino acid, at least one of the of the cysteine residues corresponding to C₁, C₂, C₃, C₄, C₅, C₆, C₇, and C₈ of the reference Defensin peptide is deleted, and wherein at least two of the cysteine residues corresponding to C₁, C₂, C₃, C₄, C₇, and C₈ of the reference Defensin peptide are retained.

50. The composition of claim 46, wherein the Defensin peptide comprises cysteine residues corresponding to C₅, C₆, C₇, and C₈ of a reference Defensin peptide.

51. The composition of claim 46, wherein said Defensin peptide is a native Defensin peptide.

52. The composition of claim 46, wherein said Defensin peptide is a synthetic variant of a native Defensin peptide comprising one or more amino acid insertions, deletions, and/or substitutions in a native Defensin peptide.

53. The composition of claim 46, wherein said substitutions in a native Defensin peptide comprise at least one of a substitution of a hydrophobic amino acid with a hydrophobic amino acid, a cationic amino acid with a cationic amino acid, or an anionic amino acid with a cationic or anionic amino acid.

54. The composition of claim 46, wherein said Defensin peptide comprises a C₅-5-6AA-C₆ motif that comprises in contiguous sequence from its N-terminal end three amino acids (A₁₄, A₁₅, A₁₆, A₁₇, A₁₈ and, optionally, A₁₉) flanked by an N-terminal C₅ and a C-terminal C₆.

55. The composition of claim 46, wherein: (i) said A₁₄ is a cationic amino acid selected from the group consisting of His (H), Arg (R), and Lys (K) or an aliphatic amino acid selected from the group consisting of Gly (G), Ala (A), Val (V), Leu (L), Ile (I), and Pro (P) or a polar neutral amino acid selected from the group consisting of Ser(S) and Thr (T); (ii) said A₁₆ is an aliphatic amino acid selected from the group consisting of Gly (G), Ala (A), Val (V), Leu (L), Ile (I), and Pro (P) or an aromatic amino acid selected from the group consisting of Phe (F), Tyr (Y), and Trp (W); and/or (iii) wherein said A₁₈ is a cationic amino acid selected from the group consisting of His (H), Arg (R), and Lys (K).

56. The composition of claim 46, wherein said Defensin peptide comprises a C₇-3AA-C₈ motif that comprises in contiguous sequence from its N-terminal end three amino acids (A₂₀, A₂₁, and A₂₂) flanked by an N-terminal C₇ and a C-terminal C₈.

57. The composition of claim 46, wherein: (i) said A₂₀ is a polar neutral amino acid selected from the group consisting of Ser(S) and Thr (T); (ii) said A₂₁ is a cationic amino acid selected from the group consisting of His (H), Arg (R), and Lys (K); and/or (iii) said A₂₂ is an aliphatic amino acid selected from the group consisting of Gly (G), Ala (A), Val (V), Leu (L), Ile (I), and Pro (P).

58. The composition of any one of claims 46 to 57, wherein said cationic Defensin peptide has an isoelectric point between 8.0 and 12.0.

59. The composition of any one of claims 46 to 57, wherein said DEF_PFV1 exhibits greater protease resistance than said DEF_PFV2.

60. The composition of any one of claims 46 to 57, wherein said DEF_PFV1 exhibits lower protease resistance than said DEF_PFV2.

61. The composition of any one of claims 46 to 57, wherein said Defensin peptide comprises an amino acid sequence having at least 70%, 75%, 80%, 85%, 90%, 95%, 97%, or 99% sequence identity to any one of SEQ ID NO: 1 to 454 or SEQ ID NO: 459 to SEQ ID NO: 472.

62. The composition of any one of claims 46 to 57, wherein said composition further comprises an agriculturally, pharmaceutically, or veterinary practicable carrier, diluent, or excipient.

63. A method for making a composition of any one of claims 46 to 57, said method comprising:
(a) separating a fraction comprising Defensin peptide folding variant 1 (DEF_PFV1) from one or more fractions comprising Defensin peptide folding variant 2 (DEF_PFV2), or DEF_PFV2 peptide fragments thereof, from a mixture comprising DEF_PFV1 and DEF_PFV2, or DEF_PFV2 peptide fragments thereof; and
(b) combining said fraction comprising DEF_PFV1, or a preparation further purified from said DEF_PFV1 fraction, with an agriculturally, pharmaceutically, or veterinary practicable carrier, diluent, or excipient, thereby making the composition.

64. The method of claim 63, further comprising obtaining said mixture comprising DEF_PFV1 and DEF_PFV2, or DEF_PFV2 peptide fragments thereof, by
(a) culturing in fermentation broth microorganisms expressing a recombinant polynucleotide comprising a transcriptional promoter operably linked to a polynucleotide encoding a signal peptide that is in frame with and upstream of a polynucleotide encoding DEF_PFV1, or a variant thereof, and
(b) separating the microorganisms from the fermentation broth, which comprises the mixture,
wherein said microorganisms are optionally selected from the group consisting of Candida, Kluveromyces, Hansenula, Pichia, Saccharomyces, Schizosaccharomyces, or Yarrowia, Myceliophthora, and Escherichia.

65. The method of claim 63, further comprising obtaining said mixture comprising DEF_PFV1 and DEF_PFV2, or DEF_PFV2 peptide fragments thereof, by
(a) culturing in fermentation broth microorganisms expressing a recombinant polynucleotide comprising a transcriptional promoter operably linked to a polynucleotide encoding DEF_PFV1, or a variant thereof, and
(b) separating the microorganisms, which comprises the mixture, from the fermentation broth,
(c) disrupting the microorganisms to obtain a cell lysate, and
(d) separating insoluble debris in the cell lysate from an aqueous fraction comprising said mixture,
wherein said microorganisms are optionally selected from the group consisting of Candida, Kluveromyces, Hansenula, Pichia, Saccharomyces, Schizosaccharomyces, or Yarrowia, Myceliophthora, and Escherichia.

66. The method of claim 63, further comprising treating said mixture comprising DEF_PFV1 and DEF_PFV2 with a protease under non-denaturing conditions that are sufficient to produce DEF_PFV2 peptide fragments but insufficient to produce DEF_PFV1 peptide fragments.

67. The method of claim 63, wherein the protease is a trypsin family serine protease that cleaves DEF_PFV2 at the carboxy terminus of a DEF_PFV2 Arg (R) or Lys (K).

68. The method of claim 63, wherein the trypsin family serine protease is a recombinant bovine, porcine, human, or microbial trypsin, and, optionally, wherein the microbial trypsin is a Streptomycete trypsin or variant thereof.

69. The method of claim 63, further comprising separating the DEF_PFV1 from the DEF_PFV2 peptide fragments by size-exclusion chromatography.

70. A method for preventing or reducing crop damage or post-harvest loss by a plant pathogenic microbe, said method comprising contacting a plant, a plant seed, pre- or post-harvest grain, pre- or post-harvest fruit, or pre- or post-harvest vegetable with an effective amount of the composition of any one of claim 1-24 or 46-57, and under conditions suitable for preventing or reducing crop damage or post-harvest loss.

71. The method of claim 70, wherein the plant pathogenic microbe is selected from the group consisting of Fusarium sp., Alternaria sp., Verticillium sp., Phytophthora sp., Colletotrichum sp., Botrytis sp., Cercospora sp., Phakopsora sp., Rhizoctonia sp., Sclerotinia sp., Pythium sp., Phoma sp., Leptosphaeria sp., Gaeumannomyces sp., Puccinia sp., Septoria sp., Penicillium sp., Lasiodiplodia sp., Phomopsis sp., Mycosphaerella sp., Golovinomyces sp., Erisyphe sp., Albugo sp., Setosphaeria sp., Cochliobolus sp., Helminthosporium sp., Diplodia sp. or Stenocarpella sp.

72. The method of claim 70, wherein the plant, plant seed, pre- or post-harvest grain, pre- or post-harvest fruit, or pre- or post-harvest vegetable is selected from the group consisting of a cereal crop, a legume crop, a root or tuber crop, an oil crop, a fruit crop, a vegetable crop, a nut crop, a forage or turf grass crop, a forage legume, a drug crop, a spice or flavoring crop, a fiber crop, and a biofuel crop plant, a plant seed, pre- or post-harvest grain, pre- or post-harvest fruit, or pre- or post-harvest vegetable.

73. The method of claim 70, wherein the plant, a plant seed, pre- or post-harvest grain, pre- or post-harvest fruit, or pre- or post-harvest vegetable is selected from the group consisting of wheat, rice, corn, barley, oat, sorghum, rye, millet, peanut, chickpea, pigeon pea, lentil, kidney bean, soybean, lima bean, potato, sweet potato, cassava, canola, peanut, palm, coconut, safflower, cotton, sunflower, flax, olive, sugarcane, sugarbeet, banana, orange, apple, pear, breadfruit, pineapple, strawberry, grape, cherry, coffee, coco, tomato, lettuce, carrot, melon, asparagus, Brassica sp., cashew, walnut, pistachio, almond, alfalfa, clover, castor, Camelina, pennycress, switchgrass, Miscanthus, and Jatropha plant, plant seed, pre- or post-harvest grain, pre- or post-harvest fruit, or pre- or post-harvest vegetable.

74. A medical device having antimicrobial properties, said medical device comprising a medical device in operable combination with a composition of any one of claim 1-24 or 46-57, wherein said medical device comprises at least one surface that is topically coated or impregnated with said composition.

75. The medical device of claim 74, wherein said medical device is selected from the group consisting of a stent, a catheter, a contact lens, a condom, a patch, and a diaphragm.

76. A method for treating, preventing, or inhibiting a microbial infection in a subject in need thereof, said method comprising administering to said subject a therapeutically effective amount of a composition of any one of claim 1-24 or 46-57 under conditions and for a duration suitable for treating, preventing, or inhibiting said microbial infection.

77. The method of claim 76, wherein said administering comprises topical, enteral, parenteral, or intravenous introduction of the composition to the subject.

78. The method of claim 76, wherein the subject is selected from the group consisting of human, livestock, poultry, fish, and companion animal.

79. The method of claim 76, wherein the microbial infection is of a mucosal membrane, eye, skin, or a nail and the composition is applied to the mucosal membrane, eye, skin, or nail.

80. The method of claim 76, wherein the microbial infection is by a dermatophyte, and wherein the dermatophyte is optionally selected from the group consisting of Trichophyton rubrum, Trichophyton interdigitale, Trichophyton violaceum, Trichophyton tonsurans, Trichophyton soudanense, Trichophyton mentagrophytes, Microsporum flavum, Epidermophyton floccosum, and Microsporum gypseum.

81. The method of claim 76, wherein the microbial infection is by a microorganism selected from the group consisting of Aspergillus, Cryptococcus, Penicillium, Rhizopus, Apophysomyces, Cunninghamella, Saksenaea, Rhizomucor, Syncephalostrum, Cokeromyces, Actinomucor, Pythium, Fusarium, Histoplasmosis, and Blastomyces species.

82. The method of claim 76, wherein the microbial infection is by a microorganism selected from the group consisting of Candida species,
wherein the Candida species is selected from the group consisting of Candida albicans (C. albicans), C. auris, C. glabrata, C. parasilosis, C. tropicalis, and C. krusei.

83. The composition of any one of claim 1-24 or 46-57 for use in a method for treating, preventing, or inhibiting microbial infection in a subject in need thereof.

84. The composition of claim 83, wherein the subject is selected from the group consisting of human, livestock, poultry, fish, and companion animals.

85. A plant part that is at least partly coated with the composition of any one of claim 1-24 or 46-57.

86. The plant part of claim 85, wherein said plant part is selected from the group consisting of a seed and a pre- or post-harvest grain.

87. The plant part of claim 85, wherein said plant part is selected from the group consisting of pre- or post-harvest fruit, pre- or post-harvest vegetable, and pre- or post-harvest flower.

88. The plant part of claim 85, wherein the plant part is selected from the group consisting of cereal crop, legume crop, root or tuber crop, oil crop, fruit crop, vegetable crop, nut crop, forage or turf grass crop, forage legume, drug crop, spice or flavoring crop, fiber crop, and biofuel crop plant part.

89. The plant part of claim 85, wherein the plant part is selected from the group consisting of wheat, rice, corn, barley, oat, sorghum, rye, millet, peanut, chickpea, pigeon pea, lentil, kidney bean, soybean, lima bean, potato, sweet potato, cassava, canola, peanut, palm, coconut, safflower, cotton, sunflower, flax, olive, sugarcane, sugarbeet, banana, orange, apple, pear, breadfruit, pineapple, strawberry, grape, cherry, coffee, coco, tomato, lettuce, carrot, melon, asparagus, Brassica sp., cashew, walnut, pistachio, almond, alfalfa, clover, castor, Camelina, pennycress, switchgrass, Miscanthus, and Jatropha plant part.
